(19) United States
(12) Patent Application Publication
(10) Pub. No.: US 2013/0165475 A1
(43) Pub. Date: Jun. 27, 2013

(54) DEUTERIUM-ENRICHED HETEROCYCLIC COMPOUNDS AS KINASE INHIBITORS

(71) Applicants: Shan Jiang, Trumbull, CT (US); Qishan Wang, Markham (CA); Xiaoyong Gao, Suzhou (CN)

(72) Inventors: Shan Jiang, Trumbull, CT (US); Qishan Wang, Markham (CA); Xiaoyong Gao, Suzhou (CN)

(73) Assignee: ASCEPION PHARMACEUTICALS, INC., Suzhou, Jiangsu (CN)

(21) Appl. No.: 13/817,505

(22) PCT Filed: Sep. 1, 2011
(86) PCT No.: PCT/CN2011/079231

§ 371 Date: Feb. 18, 2013

Related U.S. Application Data
(60) Provisional application No. 61/379,109, filed on Sep. 1, 2010.

(51) Int. Cl. C07D 401/06; C07D 471/04

(52) U.S. Cl. CPC C07D 401/06; C07D 471/04
USPC 514/300; 546/277.4; 514/339; 546/113

(57) ABSTRACT

The present invention provides deuterium-enriched heteroaryl-containing urea compounds (I) and use of the same for treating conditions mediated by protein kinase such as

[symbol]

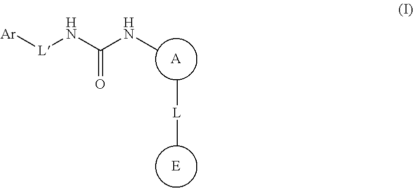

### CROSS-REFERENCE OF RELATED APPLICATION

This application claims priority to U.S. Provisional Application No. 61/379,109, filed on Sep. 1, 2010, the contents of which are incorporated herein by reference in their entirety.

### BACKGROUND OF THE INVENTION

Protein kinases constitute the largest family of human enzymes, encompassing well over 500 proteins. It has been found that kinases play a key role in many basic biological processes in the cell including but not limited to cell proliferation, survival, motility, morphogenesis, angiogenesis, and so on. In addition, many kinases were found involved and implicated in a number of pathological settings such as cancers, autoimmune and inflammatory diseases, eye diseases, and cardiovascular diseases. In general, kinases transmit cell-to-cell or intracellular signals by phosphorylating downstream proteins in the signal transduction pathways such that the downstream proteins are activated and thus signals can be passed from one step to the next down the signaling cascade. These signal transduction pathways are well regulated in the cell under normal physiological conditions. They are activated and shut down appropriately in response to the changes in the intra- and extracellular environments. However, in many pathological settings, one or more signal transduction pathways are often shown to be overactive and responsible for the occurrence and the progression of the diseases. Thus, blocking kinase function in disease settings by chemical or biological agents leading to the disruption of signaling pathways involved in the pathological processes could potentially disrupt or reduce the progression of the diseases and, therefore, confer clinical benefits to the relevant patients. Among many disease-related kinases, receptor tyrosine kinases such as c-Met (HGF/SF receptor), VEGFR2 (KDR, Flk1), PDGFRβ, and EphB4 have been well characterized and considered effective targets for therapies treating diseases such as cancers, autoimmune and inflammatory diseases, and eye diseases. See, e.g., P. Carmeliet, Nature, 2005, 438:932-936; N. Ferrara et al., Nature, 2005, 438: 967-974; P. M. Comoglio et al., Nature Reviews: Drug Discovery, 2008, 7: 504-516.

Angiogenesis, the formation of new blood vessels from preexisting ones, plays a significant role in many pathological settings, including cancer, chronic inflammation, diabetic retinopathy, psoriasis, rheumatoid arthritis, and macular degeneration. Anti-angiogenesis therapy represents an important approach for the treatment of solid tumors and other diseases associated with dysregulated vascularization. Given a continuous string of approvals of angiogenesis inhibitor drugs such as bevacizumab, sorafenib, and sunitinib for the treatment of cancers, the clinical benefit from anti-angiogenesis therapy has become increasingly evident. See, e.g., M. Atkins et al., Discovery, 2006, 5: 279-280; S. Wilhelm et al., Nature Reviews: Drug Discovery, 2006, 5: 835-844.

The process of angiogenesis requires the concerted actions of multiple angiogenesis mediators as well as the participation of different cell types. Key angiogenesis mediators have been identified, including VEGF, FGF, and angiopoietin 1 and 2 (Ang1 and Ang2) that bind to their cognate receptors (VEGFRs, FGFRs and Tie1 and Tie2, respectively) expressed on endothelial cells, as well as platelet-derived growth factor (PDGF) that binds to its receptor (PDGFRα) expressed on VEGF-producing stromal cells or its receptor (PDGFRβ) expressed on pericytes and smooth muscle cells. Molecules including VEGF, FGF, PDGF, VEGFRs, FGFRs, PDGFRs, Tie1, and Tie2 are key components of multiple different signaling pathways that function in parallel to regulate angiogenesis in both physiological and clinical settings. Among these molecules, the signal transduction pathway mediated by VEGFR2 plays the most critical role in tumor angiogenesis.

A number of monoclonal antibodies (mAbs) against single angiogenesis pathway components such as VEGF and FGF have been developed to block angiogenesis and shown to slow down tumor growth in preclinical and clinical studies. However, to a linear pathway, targeting a single component of the pathway is less effective than simultaneous blocking multiple components of the pathway. Thus development of multiplex small molecular kinase inhibitors is desirable for achieving more efficient angiogenesis inhibition. Since VEGFR2 and PDGFRβ are targeted by both sorafenib and sunitinib, the clinical benefits demonstrated in the use of both drugs unambiguously validate VEGFR2 and/or PDGFRβ kinase as effective target in the treatment of diseases such as cancer. See, e.g., M. Atkins et al., supra; S. Wilhelm et al., supra.

EphB4 tyrosine kinase is another cell surface receptor which can promote cancer cell growth in vivo by stimulating angiogenesis through its specific ligand, Ephrin-B2, in blood vessel formation. EphB4 plays a significant role in angiogenesis during the development of breast cancer and other types of cancers such as prostate cancer and gastric cancer. Upon binding to its natural ligand Ephrin-B2, EphB4 kinase is activated in tumor blood vessel formation and is also required for the formation of functional blood vessels in the developing embryo Inhibition of EphB4 in many tumor types can inhibit angiogenesis at sites of neovascularization in adults. Furthermore, disruption of interaction between EphB4 and its ligand Ephrin-B2 by monoclonal antibodies could inhibit tumor growth. Thus, sufficient experimental evidence has implicated EphB4 as a potential effective target for the treatment of diseases such as cancers. See, e.g., M. Heroult et al., Exp Cell Res, 2006, 312: 642-650; N. E B Pasquale et al., Cell, 2008, 133: 38-52; Z. Kertesz et al., Blood, 2006, 107: 2330-2338.

Numerous evidences have implicated c-Met as one of the leading molecular targets in cancer therapy. See, e.g., B. S. Knudsen et al., Current Opinion in Genetics & Development, 2008, 18: 87-96. C-Met tyrosine kinase is a cell surface receptor normally activated by its natural ligand, hepatocyte growth factor/scatter factor (HGF/SF). Activation of c-Met signaling can lead to a wide range of cellular responses including but not limited to proliferation, survival, angiogenesis, wound healing, tissue regeneration, scattering, motility and invasion. See, e.g., P. M. Comoglio et al., supra; and S. Benvenuti et al., J. Cellular Physiology, 2007, 213: 316-325. C-Met has been implicated as a proto-oncogene, which is found genomically amplified, over-expressed, mutated, or aberrantly activated in many types of cancers, suggesting its roles in the tumor growth, invasiveness and metastasis. In addition, elevated c-Met activation has been found in solid tumors which develop resistance to anti-EGFR therapies during the course of treatment, implicating a compensatory role of c-Met activation to the EGFR signaling pathway (see, e.g., G. A. Smolen et al., Proc. Natl. Acad. Sci. USA, 2006, 103: 2316-2321; B. Lutterbach et al., Cancer Res., 2007, 67: 2081-2088). Thus inhibition of c-Met signaling is considered as a potentially effective therapeutic strategy against solid tumors whose growth is wholly or partially c-Met driven (see, e.g., G. A. Smolen et al., supra).

This invention provides a solution by inhibiting one or more kinase mediated disease related signal transduction mechanisms with small molecule drugs targeting one or more protein kinases (e.g., VEGFR2, PDGFRβ, EphB4, and c-Met), which offer additional or/and better therapeutic choices and even unexpected clinical advantages over the currently available therapeutics.

### BRIEF DESCRIPTION OF THE INVENTION

One of ordinary skill in the art recognizes that most of the elements in the periodic table exist in the nature as mixtures of their isotopes. For example, hydrogen consists naturally of protium (H or 1H), deuterium (D or 2H), and trace amount of tritium (T or 3H). Protium is the most common isotope of hydrogen with a natural abundance of approximately 99.98%. Deuterium is another stable hydrogen isotope with a natural abundance of approximately 0.015%. Tritium is a radioactive hydrogen isotope with a half-life of 12.3 years. Unlike tritium, deuterium is not radioactive, and does not represent a significant toxicity hazard. Hydrogen atom actually represents a mixture of H, D, and trace amount of T with about 0.015% being D. Therefore, compounds with the level of deuterium that has been enriched to be greater than its natural abundance of 0.015% should be considered unnatural and, as a result, novel over their non-enriched counterparts.

Since carbon-deuterium (C-D) bond is much stronger than the regular carbon-protium (C—H) bond and capable of conferring a compound more resistant to metabolic degradation, incorporation of deuterium into a compound to replace the regular carbon-protium bond has the potential of improving the compound's metabolic stability without significantly affecting its biological potencies.

In a compound of this invention, when a particular position is designated as having deuterium, it is understood that the abundance of deuterium at that position is significantly greater than the natural abundance of deuterium (which is 0.015%). A position designated having deuterium typically has at least 20% (e.g., at least 50%, at least 75%, at least 90%) deuterium incorporation with the rest of being protium at that atom in this compound. All percentages given for the amount of deuterium are mole percentages.

In a compound of this invention, any atom not specifically designated as a particular isotope is meant to represent any stable, non-radioactive isotope of that atom. Unless otherwise stated, when a position is designated specifically as “H” or “hydrogen,” the position is understood to have hydrogen at its natural abundance isotopic composition.

The present invention in general provides compounds of Formula I, and methods for using these compounds for the treatment of conditions mediated by one or more protein kinases (e.g., VEGFR2, or PDGFRb, or EphB4, or c-Met or any combination of them) such as tumor, rheumatoid arthritis, autoimmune disease, acute inflammation, nephritis, diabetic retinitis, psoriasis, or macular degeneration.

In one aspect, the present invention provides compounds of Formula I, crystal forms, chelates, non-covalent complexes, prodrugs, stereoisomers, solvates, N-oxides, pharmaceutically acceptable salts, and mixtures thereof.

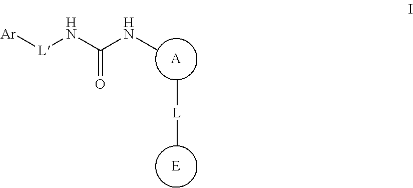

### In Formula I:

E is E1, E2 or E3:

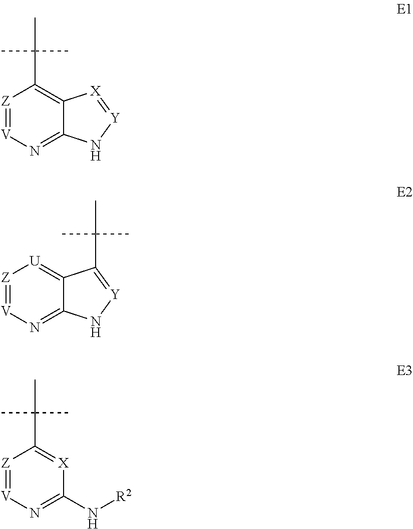

U, V, X, Y, and Z are each independently N or C—R¹;

L is a C₁₋₈alkylene, which is optionally deuterium-enriched and optionally substituted with one or more independent R³;

R² is H, C₁₋₈alkyl, C₆₋₁₂aryl, C₅₋₁₂heteroaryl, C(═O)—C₁₋₈alkyl, C(═O)—C₆₋₁₂aryl, or C(═O)—C₅₋₁₂heteroaryl, each of which is optionally substituted with one or more independent Q¹;

L′ is a covalent bond, C(═O)—, —C(═O)—C₁₋₈alkylene, or C₁₋₈alkylene, each of which is optionally substituted with one or more independent R⁴;

A is A1, A2, or A3:

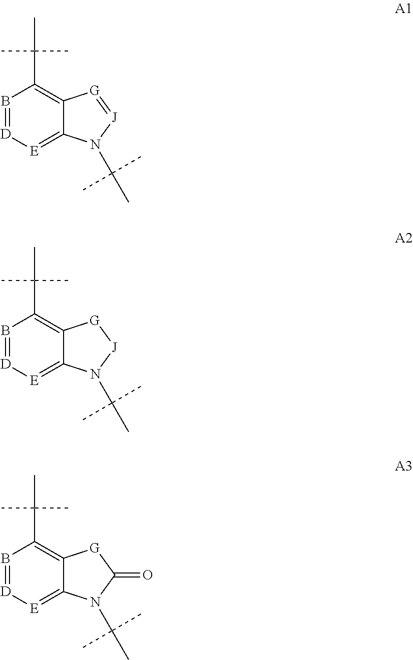

wherein B, D, E, G, and J are each independently N or CH;

each of A1, A2, and A3 is optionally substituted with one or more independent R⁵;

Ar is aryl or heteroaryl, each of which is optionally substituted with one or more independent R⁶;

R¹, R³, R⁴, R⁵, and R⁶ are each independently H, halo, —CN, —CF₃, —NO₂, —NH₂, —OH, —OCF₃, C₁₋₈alkyl-O—, —CO₂H, C₁₋₈alkyl, C₂₋₈alkenyl, C₂₋₈alkynyl, C₃₋₁₂cycloalkyl, C₃₋₁₂heterocycloalkyl, C₆₋₁₂arylC₁₋₈alkyl, or C₅₋₁₂heteroarylC₁₋₈alkyl, each of which is optionally substituted with one or more independent Q²;

or R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q²;

Q¹ and Q² are each independently H, halo, —CN, —CF₃, —OCF₃, —NO₂, oxo, C₁₋₈alkyl, C₂₋₈alkenyl, C₂₋₈alkynyl, C₃₋₁₂cycloalkyl, C₃₋₁₂heterocycloalkyl, C₆₋₁₂aryl, C₅₋₁₂heteroaryl, C₈₋₁₂heterocycloaryl, —OR⁷, —S(O)ₙR⁸, —NR⁹R¹⁰, —SO₂NR⁹R¹⁰, —C(O)R¹¹, —C(O)NR⁹R¹⁰, —C(O)OR⁷, —OC(O)R¹¹, —NR⁹C(O)R¹¹, —NR⁹S(O)₂R¹², —NR¹³C(O)NR⁹R¹⁰, —NR¹³S(O)₂NR⁹R¹⁰, or —NR¹³S(O)NR⁹R¹⁰, each of which is optionally substituted with one or more independent H, halo, —CN, —OH, —NH₂, —NO₂, oxo, —CF₃, —OCF₃, —CO₂H, —S(O)ₙH, C₁₋₈alkyl, C₆₋₁₂aryl, C₅₋₁₂heteroaryl, C₃₋₁₂cycloalkyl, C₃₋₁₂heterocycloalkyl, C₈₋₁₂heterocycloaryl, or —O—C₁₋₈alkyl, each of which is optionally partially or fully halogenated;

R⁷, R⁸, R⁹, R¹⁰, R¹¹, R¹², and R¹³ are each independently H, C₁₋₈alkyl, C₂₋₈alkenyl, C₂₋₈alkynyl, C₃₋₁₂cycloalkyl, C₃₋₁₂heterocycloalkyl, C₆₋₁₂aryl, C₅₋₁₂heteroaryl, or C₈₋₁₂heterocycloaryl;

or when in —NR⁹R¹⁰, R⁹ and R¹⁰, together with the nitrogen atom to which they are attached, form a 3- to 12-membered saturated or unsaturated ring, wherein the ring optionally includes one or more heteroatoms each independently being O, N, or S(O)ₙ;

n is 0, 1, or 2;

when L is not deuterium-enriched, then Ar must be substituted with one or more independent R⁶, and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

In some embodiments, X, Y, Z, V, and U are each independently C—R¹, thus giving compounds of formula (Ia), (Ib), or (Ic):

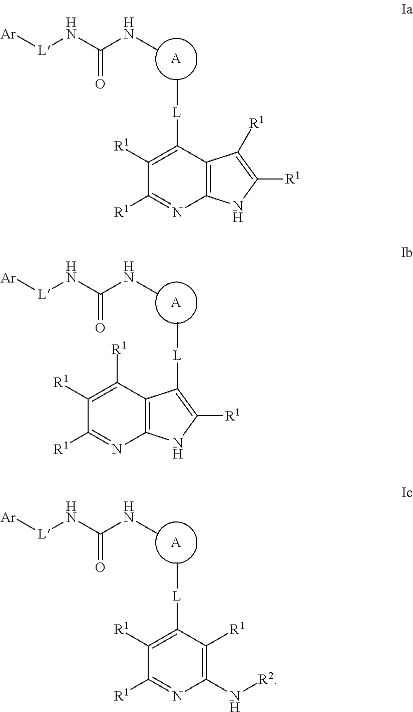

In some examples of these embodiments, L′ is a covalent bond.

In some other examples of these embodiments, Ar is C₆₋₁₂aryl or C₅₋₁₂heteroaryl, each of which is optionally substituted with one or more independent R⁶. Examples of Ar include phenyl, naphthyl, pyridinyl, pyridonyl, pyrimidinyl, pyridazinyl, triazinyl, imidazolyl, thiophenyl, furyl, thiazolyl, oxazolyl, triazolyl, quinolinyl, isoquinolinyl, tetrahydroquinolinyl, indolyl, azaindolyl, indazolyl, benzimidazolyl, benzofuryl, benzothiophenyl, benzotriazolyl, 2-oxindolyl, or indolinyl, each of which is optionally substituted with one or more independent halo, alkoxy, alkyl, haloalkoxy, cyano, oxo, or optionally substituted heterocycloalkyl.

In some other example, Ar is C₆₋₁₂aryl or C₅₋₁₂heteroaryl and is substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

In yet some other examples of these embodiments, L has 1 to 4 (e.g., 1 or 2) carbon atoms and is optionally deuterium-enriched and optionally substituted with one or more independent R³. Examples of L include —CD₂-, —CHD-, and —CD₂CD₂-.

Other examples of L include alkyl not enriched with deuterium (e.g., —CH₂—), in which case Ar is C₆₋₁₂aryl or C₅₋₁₂heteroaryl and substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

In yet still some examples, A is A1-a, A1-b, A1-c, A1-d, A1-e, A1-f, A1-g, A1-h, A1-i, A2-a, A2-b, A2-c, A2-d, A2-e, A2-f, or A2-g (shown below):

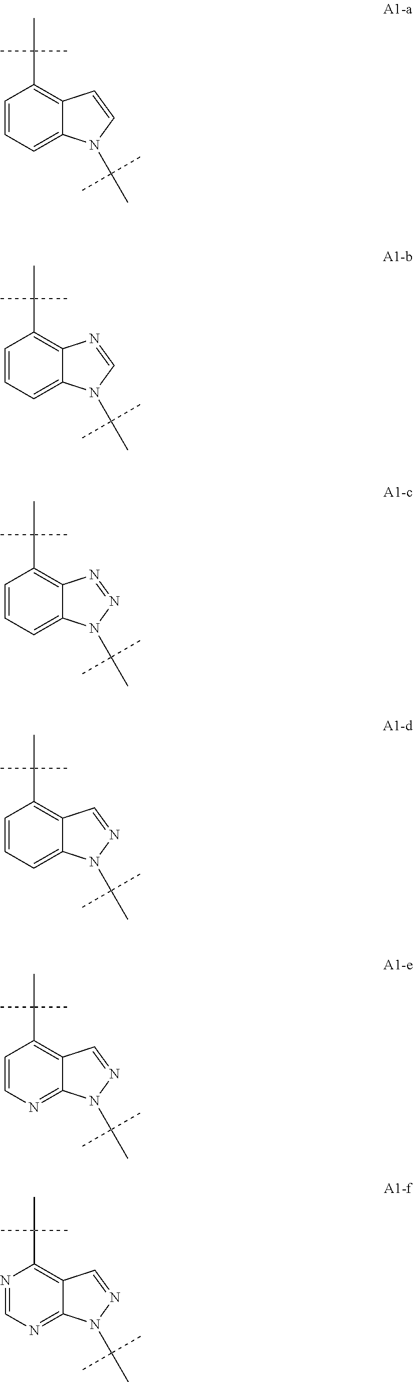

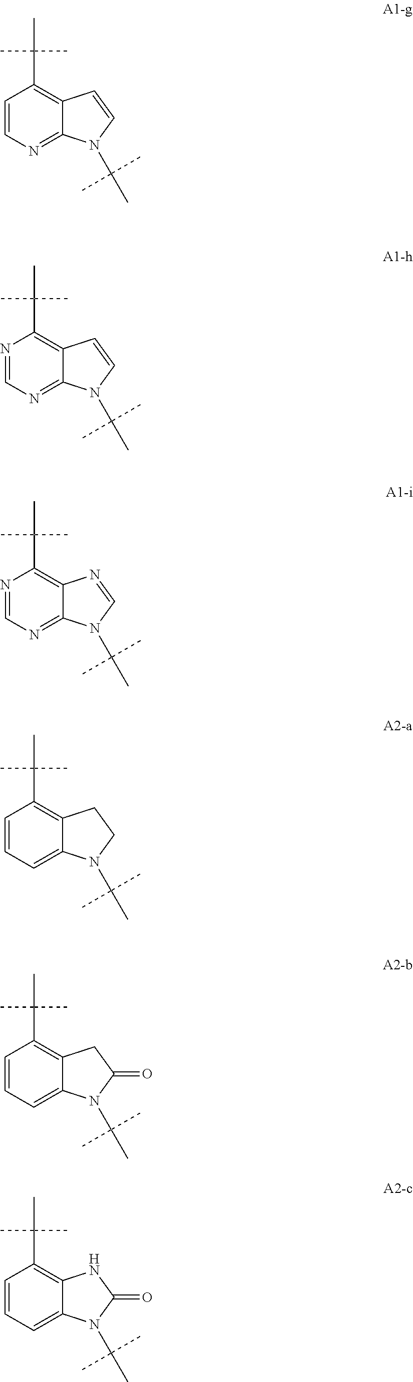

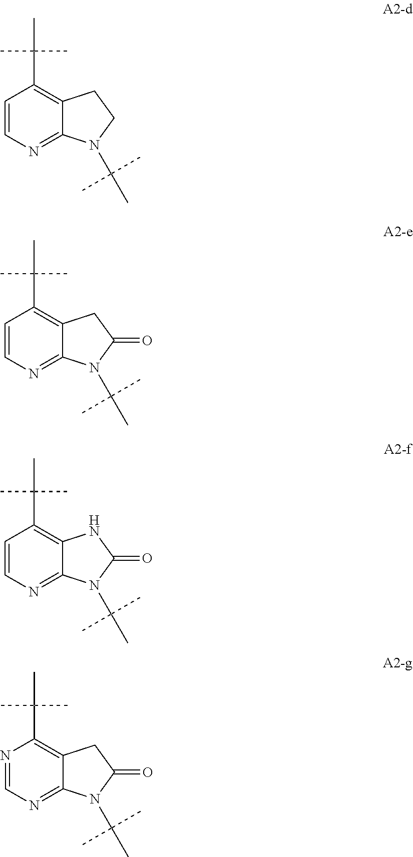

each of which is optionally substituted with one or more independent R⁵.

One group of examples of A include A1-a, A1-b, A1-d, A1-e, A1-g, A2-a, A2-b, A2-d, and A2-e, each of which is optionally substituted with one or more independent R⁵. Another group of examples of A include A1-a, A1-b, A1-d, A1-g, A2-a, and A2-d, each of which is optionally substituted with one or more independent R⁵. Still another group of examples of A include A1-a and A2-a, each of which is optionally substituted with one or more independent R⁵. A further group of examples of A include A1-a and A2-a, each of which is without optional substituents.

In some examples of these embodiments, L′ is a covalent bond; Ar is C₆₋₁₂aryl or C₅₋₁₂heteroaryl, each of which is optionally substituted with one or more independent R⁶; L has 1 to 4 carbon atoms and is optionally deuterium-enriched and optionally substituted with one or more independent R³; and A is A1-a, A1-b, A1-c, A1-d, A1-e, A1-f, A1-g, A1-h, A1-i, A2-a, A2-b, A2-c, A2-d, A2-e, A2-f, or A2-g, each of which is optionally substituted with one or more independent R⁵.

One group of examples of these embodiments include those in which L has 1 or 2 carbon atoms and is optionally deuterium-enriched and optionally substituted with one or more independent R³; and A is A1-a, A1-b, A1-d, A1-e, A1-g, A2-a, A2-b, A2-d, or A2-e, each of which is optionally substituted with one or more independent R⁵. Another group of examples of such compounds include those in which L is —CD₂-, —CHD-, or —CD₂CD₂-; and A is A1-a, A1-b, A1-d, A1-g, A2-a, or A2-d, each of which is optionally substituted with one or more independent R⁵. Still another group of examples of such compounds include those in which L is —CH₂—; Ar is C₆₋₁₂aryl or C₅₋₁₂heteroaryl and is substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

In some other embodiments, E is E1 or E2; Y is N; and X, Z, V, and U are each independently C—R¹, thus giving compounds of formula (Id) or (Ie) shown below:

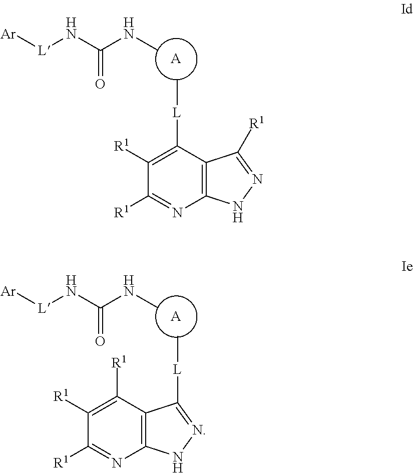

In some examples of these embodiments, L′ is a covalent bond.

In some examples of these embodiments, Ar can be a C₆₋₁₂aryl or C₅₋₁₂heteroaryl, each of which is optionally substituted with one or more independent R⁶. Examples of Ar include phenyl, naphthyl, pyridinyl, pyridonyl, pyrimidinyl, pyridazinyl, triazinyl, imidazolyl, thiophenyl, furyl, thiazolyl, oxazolyl, triazolyl, quinolinyl, isoquinolinyl, tetrahydroquinolinyl, indolyl, azaindolyl, indazolyl, benzimidazolyl, benzofuryl, benzothiophenyl, benzotriazolyl, 2-oxindolyl, or indolinyl, each of which is optionally substituted with one or more independent halo, alkoxy, alkyl, haloalkoxy, cyano, oxo, or optionally substituted heterocycloalkyl.

In some other examples, Ar is C₆₋₁₂aryl or C₅₋₁₂heteroaryl and substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

In some other examples of these embodiments, L has 1 to 4 (e.g., 1 or 2) carbon atoms and is optionally deuterium-enriched and optionally substituted with one or more independent R³. Still as an alternative, in these embodiments, L can be —CD₂-, —CHD-, or —CD₂CD₂-.

In some other examples of these embodiments, A is A1-a, A1-b, A1-c, A1-d, A1-e, A1-f, A1-g, A1-h, A1-i, A2-a, A2-b, A2-c, A2-d, A2-e, A2-f, or A2-g:

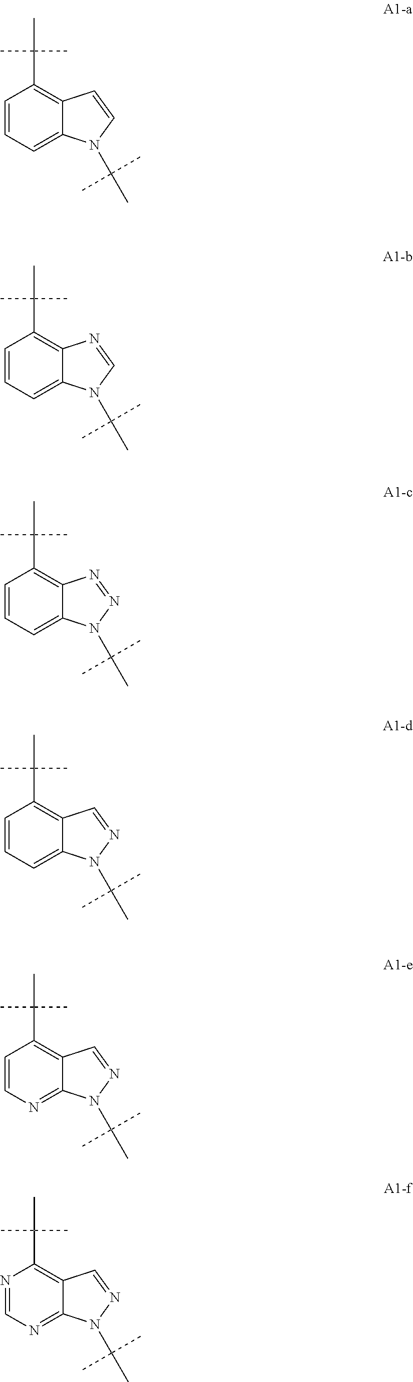

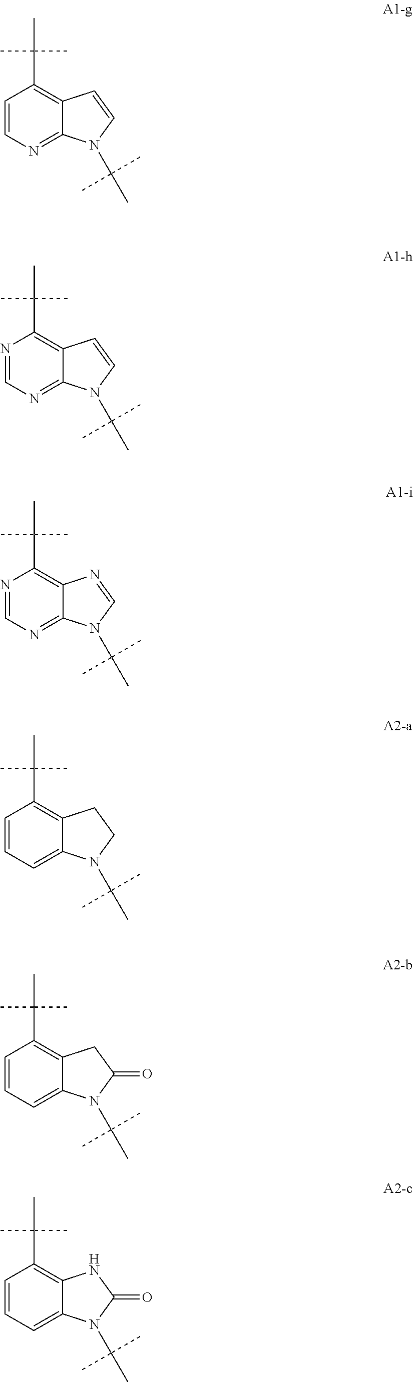

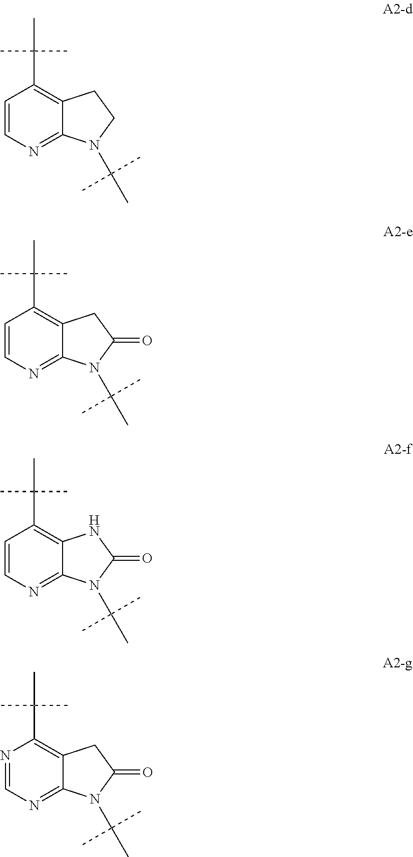

each of which is optionally substituted with one or more independent R⁵.

One group of examples of A include A1-a, A1-b, A1-d, A1-e, A1-g, A2-a, A2-b, A2-d, and A2-e, each of which is optionally substituted with one or more independent R⁵. Another group of examples of A include A1-a, A1-b, A1-d, A1-g, A2-a, or A2-d, each of which is optionally substituted with one or more independent R⁵. Still another group of examples of A include A1-a and A2-a, each of which is optionally substituted with one or more independent R⁵. A further group of examples of A include A1-a and A2-a, each of which is without optional substituents.

In yet some further some examples of these embodiments, L′ is a covalent bond; Ar is C₆₋₁₂aryl or C₅₋₁₂heteroaryl, each of which is optionally substituted with one or more independent R⁶; L has 1 to 4 carbon atoms and is optionally substituted with one or more independent R³; and A is A1-a, A1-b, A1-c, A1-d, A1-e, A1-f, A1-g, A1-h, A1-i, A2-a, A2-b, A2-c, A2-d, A2-e, A2-f, or A2-g, each of which is optionally substituted with one or more independent R⁵. One group of examples of such embodiments include compounds of Formula I in which L has 1 or 2 carbon atoms and is optionally deuterium-enriched and optionally substituted with one or more independent R³; and A is A1-a, A1-b, A1-d, A1-e, A1-g, A2-a, A2-b, A2-d, or A2-e, each of which is optionally substituted with one or more independent R⁵. Another group of examples include the compounds in which L is —CD₂-, —CHD-, or —CD₂CD₂-; and A is A1-a, A1-b, A1-d, A1-g, A2-a, or A2-d, each of which is optionally substituted with one or more independent R⁵. Still another group of examples include those compounds in which L is —CH₂—; Ar is C₆₋₁₂aryl or C₅₋₁₂heteroaryl and substituted with one or more independent R⁶; and at least one R⁶ is deuterium-enriched C₁₋₈alkyl, deuterium-enriched C₃₋₁₂ cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

In some other embodiments of the compounds of this invention, Z is N; and X, Y, V, and U are each independently C—R¹, thus giving compounds of formula (If), (Ig), or (Ih) shown below:

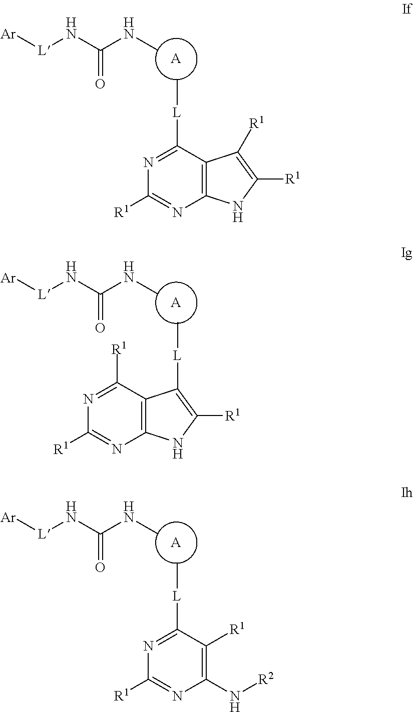

In some examples of these embodiments, L′ is a covalent bond. In some other examples, Ar is C₆₋₁₂aryl or C₅₋₁₂heteroaryl, each of which is optionally substituted with one or more independent R⁶. In still some examples, Ar is substituted with one or more independent R⁶; and at least one R⁶ is deuterium-enriched C₁₋₈alkyl, deuterium-enriched C₃₋₁₂ cycloalkyl, deuterium-enriched C₃₋₁₂ heterocycloalkyl, deuterium-enriched C₁₋₈ alkyl-O—, deuterium-enriched C₃₋₁₂cycloalkyl-O—, or deuterium-enriched C₃₋₁₂heterocycloalkyl-O—, each of which is optionally substituted with one or more independent Q². In yet still some other examples, L has 1 to 4 (e.g., 1 or 2) carbon atoms and is optionally deuterium-enriched and optionally substituted with one or more independent R³. Specific examples of L include —CD₂-, —CHD-, and —CD₂CD₂-. Other examples of L include alkyl not enriched with deuterium (e.g., —CH₂—), in which case Ar is C₆₋₁₂aryl or C₅₋₁₂heteroaryl and substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

In some other examples of these embodiments, A is A1-a, A1-b, A1-c, A1-d, A1-e, A1-f, A1-g, A1-h, A1-i, A2-a, A2-b, A2-c, A2-d, A2-e, A2-f, or A2-g:

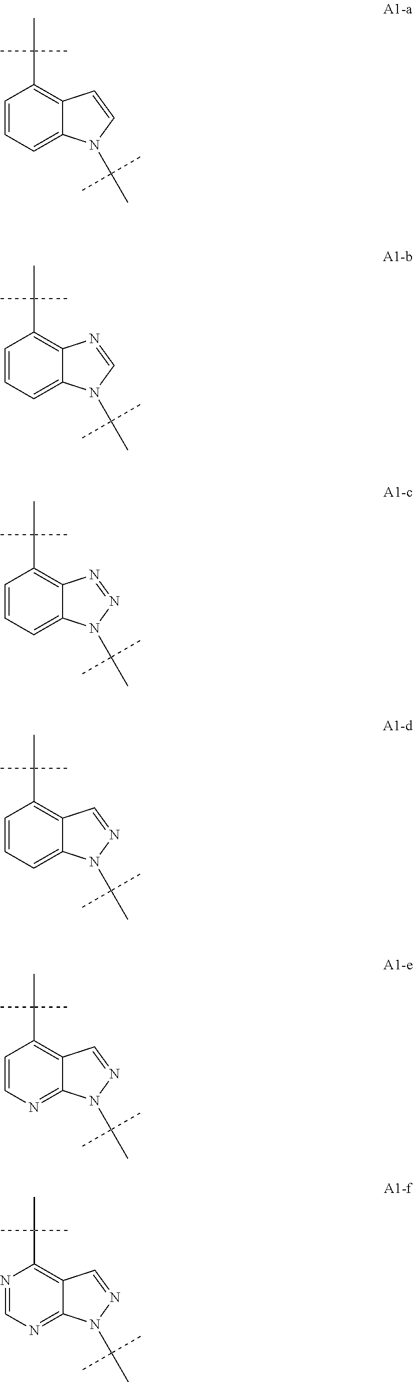

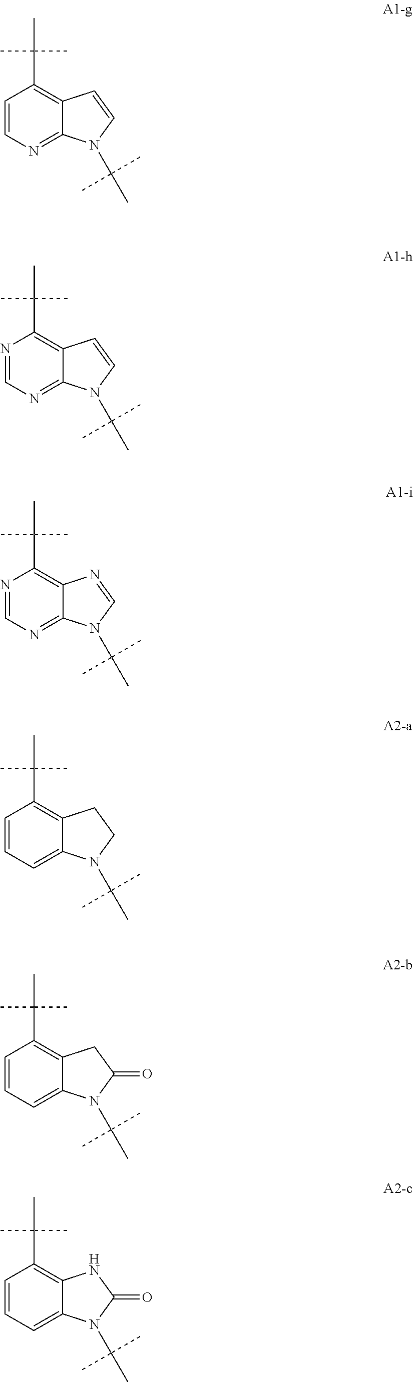

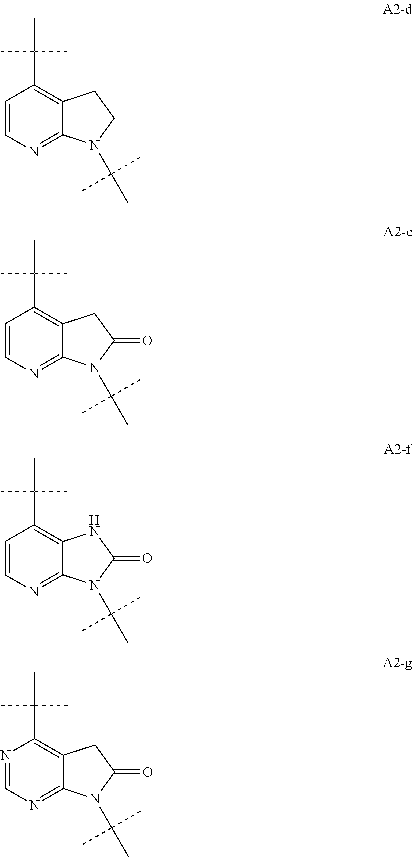

each of which is optionally substituted with one or more independent R⁵. One group of examples of A include A1-a, A1-b, A1-d, A1-e, A1-g, A2-a, A2-b, A2-d, and A2-e, each of which is optionally substituted with one or more independent R⁵. Another group of examples of A include A1-a, A1-b, A1-d, A1-g, A2-a, and A2-d, each of which is optionally substituted with one or more independent R⁵. Still another group of examples of A include A1-a and A2-a, each of which is optionally substituted with one or more independent R⁵. A further group of examples of A include A1-a and A2-a, each of which is without optional substituents.

In some examples of the embodiments described above, L′ is a covalent bond; Ar is C₆₋₁₂aryl or C₅₋₁₂heteroaryl, each of which is optionally substituted with one or more independent R⁶; L has 1 to 4 carbon atoms and is optionally deuterium-enriched and optionally substituted with one or more independent R³; and A is A1-a, A1-b, A1-c, A1-d, A1-e, A1-f, A1-g, A1-h, A1-i, A2-a, A2-b, A2-c, A2-d, A2-e, A2-f, or A2-g, each of which is optionally substituted with one or more independent R⁵. In some other examples of the compounds, L has 1 or 2 carbon atoms and is optionally deuterium-enriched and optionally substituted with one or more independent R³; and A is A1-a, A1-b, A1-d, A1-e, A1-g, A2-a, A2-b, A2-d, or A2-e, each of which is optionally substituted with one or more independent R⁵. In yet still some other examples, L is —CD₂-, —CHD-, or —CD₂CD₂-; and A is A1-a, A1-b, A1-d, A1-g, A2-a, or A2-d, each of which is optionally substituted with one or more independent R⁵. In some other examples, L include alkyl not enriched with deuterium (e.g., —CH₂—), in which case Ar is C₆₋₁₂aryl or C₅₋₁₂heteroaryl and substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

In some other embodiments of the compounds of this invention, X is N; and Y, Z, and V are each independently C—R¹, thus giving compounds of formula (II) or (Ij):

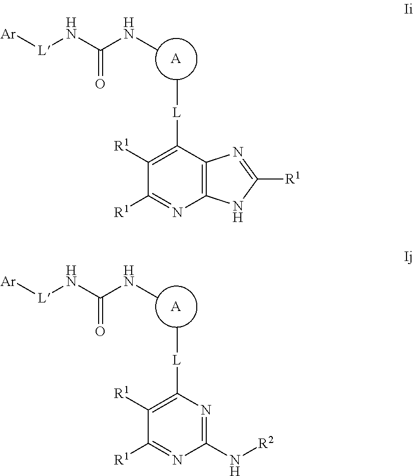

In some examples of these embodiments, L′ is a covalent bond.

In some other examples of these embodiments, Ar is C₆₋₁₂aryl or C₅₋₁₂heteroaryl, each of which is optionally substituted with one or more independent R⁶. Examples of Ar include phenyl, naphthyl, pyridinyl, pyridonyl, pyrimidinyl, pyridazinyl, triazinyl, imidazolyl, thiophenyl, furyl, thiazolyl, oxazolyl, triazolyl, quinolinyl, isoquinolinyl, tetrahydroquinolinyl, indolyl, azaindolyl, indazolyl, benzimidazolyl, benzofuryl, benzothiophenyl, benzotriazolyl, 2-oxindolyl, or indolinyl, each of which is optionally substituted with one or more independent halo, alkoxy, alkyl, haloalkoxy, cyano, oxo, or optionally substituted heterocycloalkyl. In still some examples, Ar is substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

In yet some other examples of these embodiments, L has 1 to 4 (e.g., 1 or 2) carbon atoms and is optionally deuterium-enriched and optionally substituted with one or more independent R³. Specific examples of L include —CD₂-, —CHD-, and —CD₂CD₂-. In some other examples, L include alkyl not enriched with deuterium (e.g., —CH₂—), in which case Ar is C₆₋₁₂aryl or C₅₋₁₂heteroaryl and substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

In yet still some other examples of these embodiments, A is A1-a, A1-b, A1-c, A1-d, A1-e, A1-f, A1-g, A1-h, A1-i, A2-a, A2-b, A2-c, A2-d, A2-e, A2-f, or A2-g:

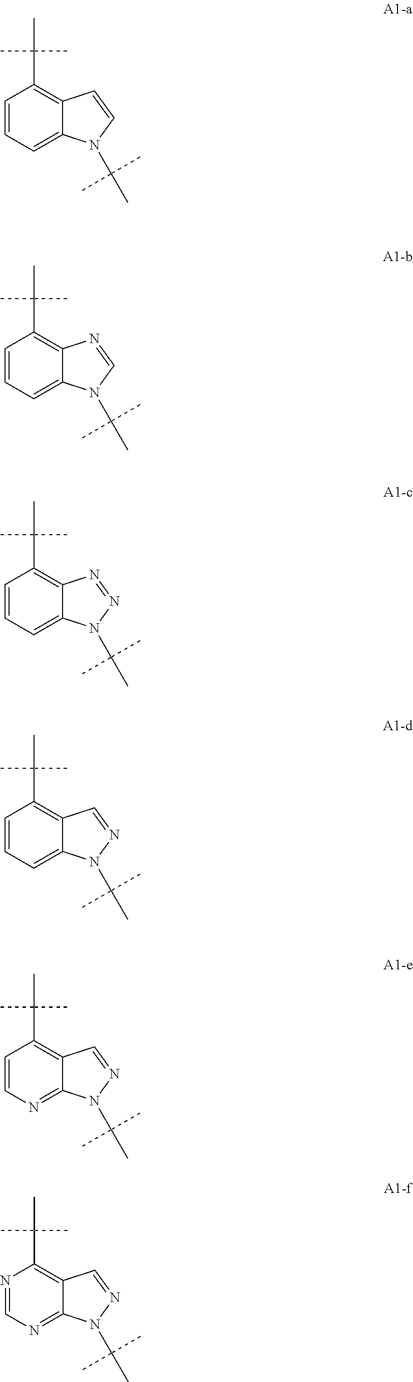

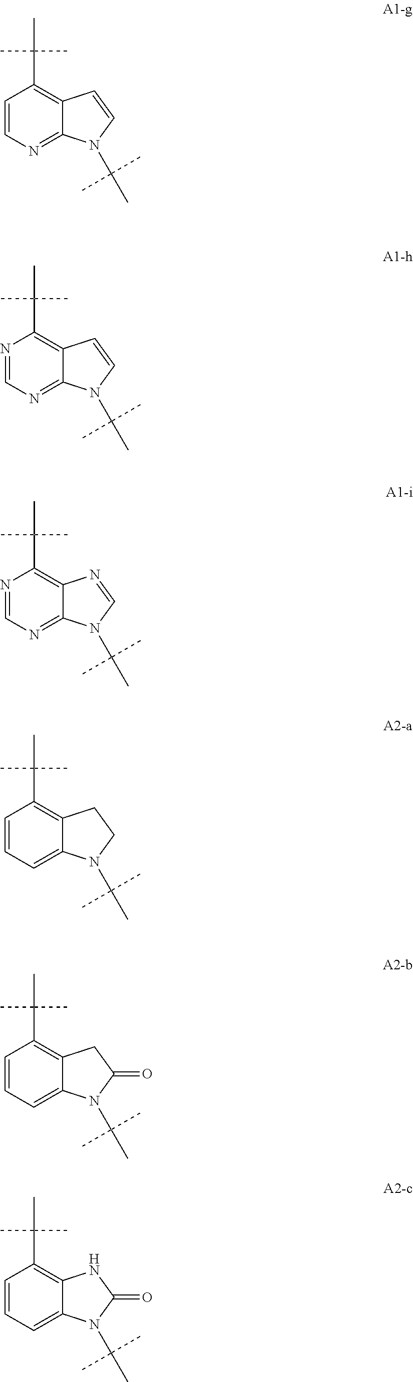

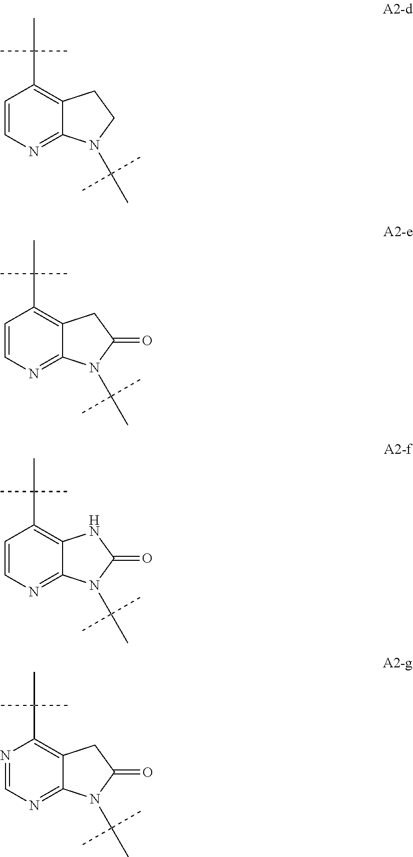

each of which is optionally substituted with one or more independent R⁵.

One group of examples of A include A1-a, A1-b, A1-d, A1-e, A1-g, A2-a, A2-b, A2-d, and A2-e, each of which is optionally substituted with one or more independent R⁵. Another group of examples of A include A1-a, A1-b, A1-d, A1-g, A2-a, and A2-d, each of which is optionally substituted with one or more independent R⁵. Still another group of examples of A include A1-a and A2-a, each of which is optionally substituted with one or more independent R⁵. A further group of examples of A include A1-a and A2-a, each of which is without optional substituents.

In some examples of the embodiments, L′ is a covalent bond; Ar is C₆₋₁₂aryl or C₅₋₁₂heteroaryl, each of which is optionally substituted with one or more independent R⁶; L has 1 to 4 carbon atoms and is optionally deuterium-enriched and optionally substituted with one or more independent R³; and A is A1-a, A1-b, A1-c, A1-d, A1-e, A1-f, A1-g, A1-h, A1-i, A2-a, A2-b, A2-c, A2-d, A2-e, A2-f, or A2-g, each of which is optionally substituted with one or more independent R⁵.

In some other examples, L has 1 or 2 carbon atoms and is optionally deuterium-enriched and optionally substituted with one or more independent R³; and A is A1-a, A1-b, A1-d, A1-e, A1-g, A2-a, A2-b, A2-d, or A2-e, each of which is optionally substituted with one or more independent R⁵.

In yet still some other examples, L is —CD₂-, —CHD-, or —CD₂CD₂-; and A is A1-a, A1-b, A1-d, A1-g, A2-a, or A2-d, each of which is optionally substituted with one or more independent R⁵. In some other examples, L is —CH₂—, and Ar is C₆₋₁₂aryl or C₅₋₁₂heteroaryl and substituted with one or more independent R⁶; and at least one R⁶ is deuterium-enriched C₁₋₈ alkyl, deuterium-enriched C₃₋₁₂ cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

In some other embodiments of the compounds of this invention, X and Z are N; and Y and V are each independently C—R¹, thus giving compounds of formula (Ik) or (Il):

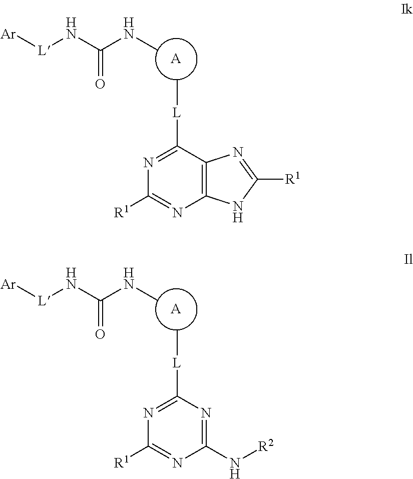

In some examples of these embodiments, L′ is a covalent bond.

In some other examples of these embodiments, Ar is C₆₋₁₂aryl or C₅₋₁₂heteroaryl, each of which is optionally substituted with one or more independent R⁶. Examples of A include phenyl, naphthyl, pyridinyl, pyridonyl, pyrimidinyl, pyridazinyl, triazinyl, imidazolyl, thiophenyl, furyl, thiazolyl, oxazolyl, triazolyl, quinolinyl, isoquinolinyl, tetrahydroquinolinyl, indolyl, azaindolyl, indazolyl, benzimidazolyl, benzofuryl, benzothiophenyl, benzotriazolyl, 2-oxindolyl, or indolinyl, each of which is optionally substituted with one or more independent halo, alkoxy, alkyl, haloalkoxy, cyano, oxo, or optionally substituted heterocycloalkyl. In still some examples, Ar is substituted with one or more independent R⁶; and at least one R⁶ is deuterium-enriched C₁₋₈alkyl, deuterium-enriched C₃₋₁₂ cycloalkyl, deuterium-enriched C₃₋₁₂heterocycloalkyl, deuterium-enriched C₁₋₈ alkyl-O—, deuterium-enriched C₃₋₁₂cycloalkyl-O—, or deuterium-enriched C₃₋₁₂heterocycloalkyl-O—, each of which is optionally substituted with one or more independent Q².

In yet still some other examples, L has 1 to 4 (e.g., 1 or 2) carbon atoms and is optionally deuterium-enriched and optionally substituted with one or more independent R³. Specific examples of L include —CD₂-, —CHD-, and —CD₂CD₂-. In some other examples, L is —CH₂—, and Ar is C₆₋₁₂aryl or C₅₋₁₂heteroaryl and substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

In still some further examples of the embodiments, A is A1-a, A1-b, A1-c, A1-d, A1-e, A1-f, A1-g, A1-h, A1-i, A2-a, A2-b, A2-c, A2-d, A2-e, A2-f, or A2-g:

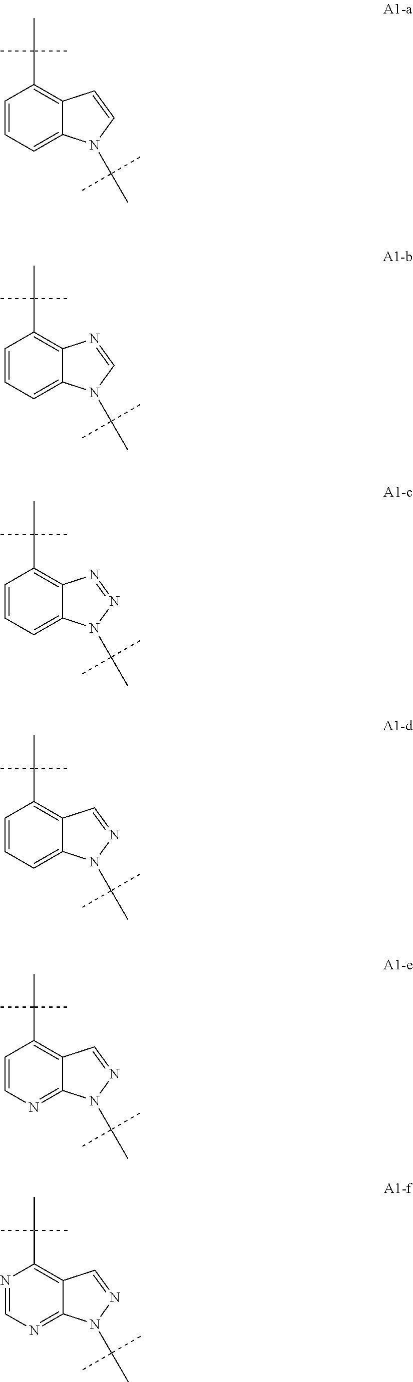

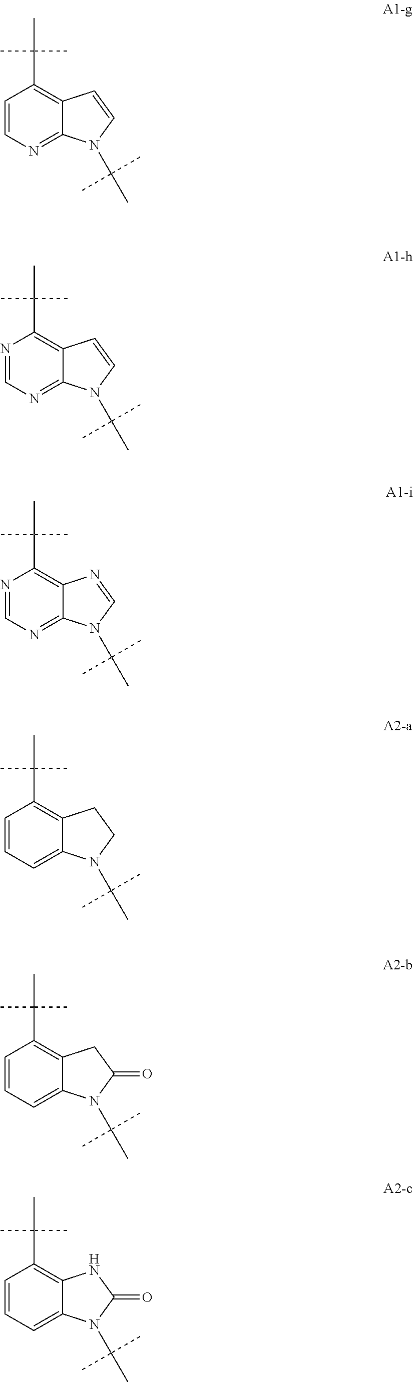

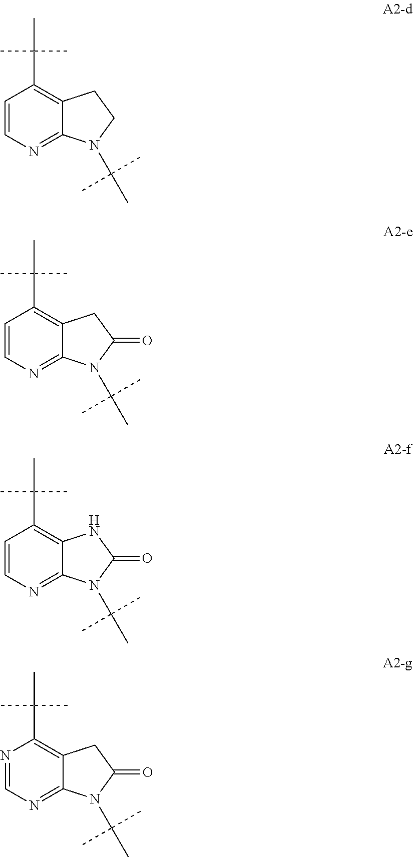

each of which is optionally substituted with one or more independent R⁵.

One group of examples of A include A1-a, A1-b, A1-d, A1-e, A1-g, A2-a, A2-b, A2-d, and A2-e, each of which is optionally substituted with one or more independent R⁵. Another group of examples of A include A1-a, A1-b, A1-d, A1-g, A2-a, and A2-d, each of which is optionally substituted with one or more independent R⁵. Still another group of examples of A include A1-a and A2-a, each of which is optionally substituted with one or more independent R⁵. A further group of examples of A include A1-a and A2-a, each of which is without optional substituents.

In yet still some examples of the embodiments, L′ is a covalent bond; Ar is C₆₋₁₂aryl or C₅₋₁₂heteroaryl, each of which is optionally substituted with one or more independent R⁶; L has 1 to 4 carbon atoms and is optionally deuterium-enriched and optionally substituted with one or more independent R³; and A is A1-a, A1-b, A1-c, A1-d, A1-e, A1-f, A1-g, A1-h, A1-i, A2-a, A2-b, A2-c, A2-d, A2-e, A2-f, or A2-g, each of which is optionally substituted with one or more independent R⁵.

In some other examples of these embodiments, L has 1 or 2 carbon atoms and is optionally deuterium-enriched and optionally substituted with one or more independent R³; and A is A1-a, A1-b, A1-d, A1-e, A1-g, A2-a, A2-b, A2-d, or A2-e, each of which is optionally substituted with one or more independent R⁵. In alternative examples, L is —CD₂-, —CHD-, or —CD₂CD₂-; and A is A1-a, A1-b, A1-d, A1-g, A2-a, or A2-d, each of which is optionally substituted with one or more independent R⁵. In some other examples, L is —CH₂—, and Ar is C₆₋₁₂aryl or C₅₋₁₂heteroaryl and substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

In some embodiments, the compounds of this invention are of the structure:

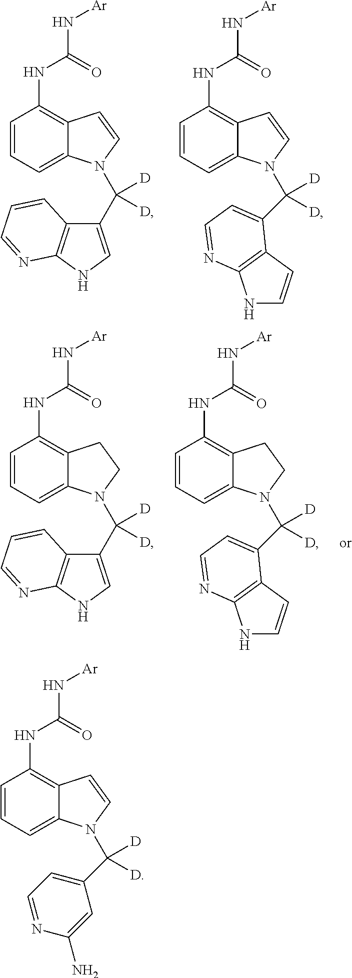

In some other embodiments, the compounds of this invention are of the structure:

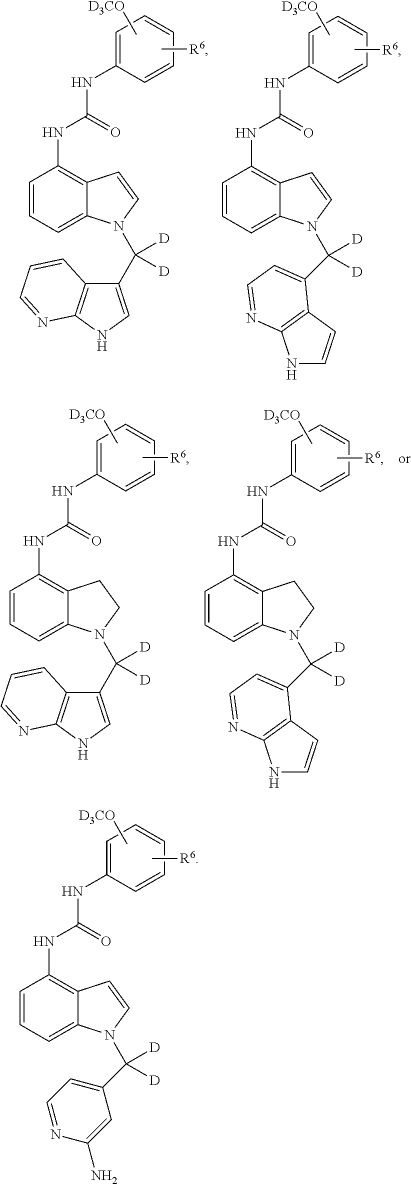

In some embodiments, Ar is phenyl, naphthyl, pyridinyl, pyridonyl, pyrimidinyl, pyridazinyl, triazinyl, imidazolyl, thiophenyl, furyl, thiazolyl, oxazolyl, triazolyl, quinolinyl, isoquinolinyl, tetrahydroquinolinyl, indolyl, azaindolyl, indazolyl, benzimidazolyl, benzofuryl, benzothiophenyl, benzotriazolyl, 2-oxindolyl, or indolinyl, and is optionally substituted with one or more independent halo, alkoxy, alkyl, haloalkoxy, cyano, oxo, or optionally substituted heterocycloalkyl. In still some examples, Ar is substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

In some embodiments, the compound is:
1-[1-[dideuterio(1H-pyrrolo[2,3-b]pyridin-3-yl)methyl]indol-4-yl]-3-phenylurea;
1-[1-[dideuterio(1H-pyrrolo[2,3-b]pyridin-3-yl)methyl]indol-4-yl]-3-(2-fluorophenyl)urea;
1-[1-[dideuterio(1H-pyrrolo[2,3-b]pyridin-3-yl)methyl]indol-4-yl]-3-(3-fluorophenyl)urea;
1-[1-[dideuterio(1H-pyrrolo[2,3-b]pyridin-3-yl)methyl]indol-4-yl]-3-(4-fluorophenyl)urea;
1-[1-[dideuterio(1H-pyrrolo[2,3-b]pyridin-3-yl)methyl]indol-4-yl]-3-(2-chlorophenyl)urea;
1-[1-[dideuterio(1H-pyrrolo[2,3-b]pyridin-3-yl)methyl]indol-4-yl]-3-(3-chlorophenyl)urea;
1-[1-[dideuterio(1H-pyrrolo[2,3-b]pyridin-3-yl)methyl]indol-4-yl]-3-(4-chlorophenyl)urea;
1-[1-[dideuterio(1H-pyrrolo[2,3-b]pyridin-3-yl)methyl]indol-4-yl]-3-(2-bromophenyl)urea;
1-[1-[dideuterio(1H-pyrrolo[2,3-b]pyridin-3-yl)methyl]indol-4-yl]-3-(3-bromophenyl)urea;
1-[1-[dideuterio(1H-pyrrolo[2,3-b]pyridin-3-yl)methyl]indol-4-yl]-3-(4-bromophenyl)urea;
1-[1-[dideuterio(1H-pyrrolo[2,3-b]pyridin-3-yl)methyl]indol-4-yl]-3-(2-methylphenyl)urea;
1-[1-[dideuterio(1H-pyrrolo[2,3-b]pyridin-3-yl)methyl]indol-4-yl]-3-(3-methylphenyl)urea;
1-[1-[dideuterio(1H-pyrrolo[2,3-b]pyridin-3-yl)methyl]indol-4-yl]-3-(4-methylphenyl)urea;
1-[1-[dideuterio(1H-pyrrolo[2,3-b]pyridin-3-yl)methyl]indol-4-yl]-3-(2-methoxyphenyl)urea;
1-[1-[dideuterio(1H-pyrrolo[2,3-b]pyridin-3-yl)methyl]indol-4-yl]-3-(3-methoxyphenyl)urea;
1-[1-[dideuterio(1H-pyrrolo[2,3-b]pyridin-3-yl)methyl]indol-4-yl]-3-(4-methoxyphenyl)urea;
1-[1-[dideuterio(1H-pyrrolo[2,3-b]pyridin-3-yl)methyl]indol-4-yl]-3-(2-trifluoromethoxyphenyl)urea;
1-[1-[dideuterio(1H-pyrrolo[2,3-b]pyridin-3-yl)methyl]indol-4-yl]-3-(3-trifluoromethoxyphenyl)urea;
1-[1-[dideuterio(1H-pyrrolo[2,3-b]pyridin-3-yl)methyl]indol-4-yl]-3-(4-trifluoromethoxyphenyl)urea;
1-[1-[dideuterio(1H-pyrrolo[2,3-b]pyridin-3-yl)methyl]indol-4-yl]-3-(2-trifluoromethylphenyl)urea;
1-[1-[dideuterio(1H-pyrrolo[2,3-b]pyridin-3-yl)methyl]indol-4-yl]-3-(3-trifluoromethylphenyl)urea;
1-[1-[dideuterio(1H-pyrrolo[2,3-b]pyridin-3-yl)methyl]indol-4-yl]-3-(4-trifluoromethylphenyl)urea;
1-[1-[dideuterio(1H-pyrrolo[2,3-b]pyridin-3-yl)methyl]indol-4-yl]-3-(2-cyanophenyl)urea;
1-[1-[dideuterio(1H-pyrrolo[2,3-b]pyridin-3-yl)methyl]indol-4-yl]-3-(3-cyanophenyl)urea;
1-[1-[dideuterio(1H-pyrrolo[2,3-b]pyridin-3-yl)methyl]indol-4-yl]-3-(4-cyanophenyl)urea;
1-[1-[dideuterio(1H-pyrrolo[2,3-b]pyridin-3-yl)methyl]indol-4-yl]-3-(5-fluoro-2-methoxyphenyl)urea;
1-[1-[dideuterio(1H-pyrrolo[2,3-b]pyridin-3-yl)methyl]indol-4-yl]-3-(5-chloro-2-methoxyphenyl)urea;
1-[1-[dideuterio(1H-pyrrolo[2,3-b]pyridin-3-yl)methyl]indol-4-yl]-3-(5-bromo-2-methoxyphenyl)urea;
1-[1-[dideuterio(1H-pyrrolo[2,3-b]pyridin-3-yl)methyl]indol-4-yl]-3-(5-methyl-2-methoxyphenyl)urea;
1-[1-[dideuterio(1H-pyrrolo[2,3-b]pyridin-3-yl)methyl]indol-4-yl]-3-(2,5-dimethoxyphenyl)urea;
1-[1-[dideuterio(1H-pyrrolo[2,3-b]pyridin-3-yl)methyl]indol-4-yl]-3-(5-trifluoromethyl-2-methoxyphenyl)urea;
1-[1-[dideuterio(1H-pyrrolo[2,3-b]pyridin-3-yl)methyl]indol-4-yl]-3-(5-methyl-2-fluorophenyl)urea;
1-[1-[dideuterio(1H-pyrrolo[2,3-b]pyridin-3-yl)methyl]indol-4-yl]-3-(5-methoxyl-2-fluorophenyl)urea;
1-[1-[dideuterio(1H-pyrrolo[2,3-b]pyridin-3-yl)methyl]indol-4-yl]-3-(5-trifluoromethyl-2-fluorophenyl)urea;
1-[1-[dideuterio(1H-pyrrolo[2,3-b]pyridin-3-yl)methyl]indol-4-yl]-3-(5-chloro-2-fluorophenyl)urea;
1-[1-[dideuterio(1H-pyrrolo[2,3-b]pyridin-3-yl)methyl]indol-4-yl]-3-(5-bromo-2-fluorophenyl)urea; or
1-[1-[dideuterio(1H-pyrrolo[2,3-b]pyridin-3-yl)methyl]indol-4-yl]-3-(5-methyl-2-trifluoromethylphenyl)urea.

In some embodiments, the compound is
1-[1-[dideuterio(1H-pyrrolo[2,3-b]pyridin-4-yl)methyl]indol-4-yl]-3-phenylurea;
1-[1-[dideuterio(1H-pyrrolo[2,3-b]pyridin-4-yl)methyl]indol-4-yl]-3-(2-fluorophenyl)urea;
1-[1-[dideuterio(1H-pyrrolo[2,3-b]pyridin-4-yl)methyl]indol-4-yl]-3-(3-fluorophenyl)urea;
1-[1-[dideuterio(1H-pyrrolo[2,3-b]pyridin-4-yl)methyl]indol-4-yl]-3-(4-fluorophenyl)urea;
1-[1-[dideuterio(1H-pyrrolo[2,3-b]pyridin-4-yl)methyl]indol-4-yl]-3-(2-chlorophenyl)urea;
1-[1-[dideuterio(1H-pyrrolo[2,3-b]pyridin-4-yl)methyl]indol-4-yl]-3-(3-chlorophenyl)urea;
1-[1-[dideuterio(1H-pyrrolo[2,3-b]pyridin-4-yl)methyl]indol-4-yl]-3-(4-chlorophenyl)urea;
1-[1-[dideuterio(1H-pyrrolo[2,3-b]pyridin-4-yl)methyl]indol-4-yl]-3-(2-bromophenyl)urea;
1-[1-[dideuterio(1H-pyrrolo[2,3-b]pyridin-4-yl)methyl]indol-4-yl]-3-(3-bromophenyl)urea;
1-[1-[dideuterio(1H-pyrrolo[2,3-b]pyridin-4-yl)methyl]indol-4-yl]-3-(4-bromophenyl)urea;
1-[1-[dideuterio(1H-pyrrolo[2,3-b]pyridin-4-yl)methyl]indol-4-yl]-3-(2-methylphenyl)urea;
1-[1-[dideuterio(1H-pyrrolo[2,3-b]pyridin-4-yl)methyl]indol-4-yl]-3-(3-methylphenyl)urea;
1-[1-[dideuterio(1H-pyrrolo[2,3-b]pyridin-4-yl)methyl]indol-4-yl]-3-(4-methylphenyl)urea;
1-[1-[dideuterio(1H-pyrrolo[2,3-b]pyridin-4-yl)methyl]indol-4-yl]-3-(2-methoxyphenyl)urea;
1-[1-[dideuterio(1H-pyrrolo[2,3-b]pyridin-4-yl)methyl]indol-4-yl]-3-(3-methoxyphenyl)urea;
1-[1-[dideuterio(1H-pyrrolo[2,3-b]pyridin-4-yl)methyl]indol-4-yl]-3-(4-methoxyphenyl)urea;
1-[1-[dideuterio(1H-pyrrolo[2,3-b]pyridin-4-yl)methyl]indol-4-yl]-3-(2-trifluoromethoxyphenyl)urea;
1-[1-[dideuterio(1H-pyrrolo[2,3-b]pyridin-4-yl)methyl]indol-4-yl]-3-(3-trifluoromethoxyphenyl)urea;
1-[1-[dideuterio(1H-pyrrolo[2,3-b]pyridin-4-yl)methyl]indol-4-yl]-3-(4-trifluoromethoxyphenyl)urea;
1-[1-[dideuterio(1H-pyrrolo[2,3-b]pyridin-4-yl)methyl]indol-4-yl]-3-(2-trifluoromethylphenyl)urea;
1-[1-[dideuterio(1H-pyrrolo[2,3-b]pyridin-4-yl)methyl]indol-4-yl]-3-(3-trifluoromethylphenyl)urea;
1-[1-[dideuterio(1H-pyrrolo[2,3-b]pyridin-4-yl)methyl]indol-4-yl]-3-(4-trifluoromethylphenyl)urea;
1-[1-[dideuterio(1H-pyrrolo[2,3-b]pyridin-4-yl)methyl]indol-4-yl]-3-(2-cyanophenyl)urea;
1-[1-[dideuterio(1H-pyrrolo[2,3-b]pyridin-4-yl)methyl]indol-4-yl]-3-(3-cyanophenyl)urea;
1-[1-[dideuterio(1H-pyrrolo[2,3-b]pyridin-4-yl)methyl]indol-4-yl]-3-(4-cyanophenyl)urea;
1-[1-[dideuterio(1H-pyrrolo[2,3-b]pyridin-4-yl)methyl]indol-4-yl]-3-(5-fluoro-2-methoxyphenyl)urea;
1-[1-[dideuterio(1H-pyrrolo[2,3-b]pyridin-4-yl)methyl]indol-4-yl]-3-(5-chloro-2-methoxyphenyl)urea;
1-[5-chloro-2-(trideuteriomethoxy)phenyl]-3-[1-[dideuterio(1H-pyrrolo[2,3-b]pyridin-4-yl)methyl]indol-4-yl]urea hydrochloride;
1-[1-[dideuterio(1H-pyrrolo[2,3-b]pyridin-4-yl)methyl]indol-4-yl]-3-(5-bromo-2-methoxyphenyl)urea;
1-[1-[dideuterio(1H-pyrrolo[2,3-b]pyridin-4-yl)methyl]indol-4-yl]-3-(5-methyl-2-methoxyphenyl)urea;
1-[1-[dideuterio(1H-pyrrolo[2,3-b]pyridin-4-yl)methyl]indol-4-yl]-3-(2,5-dimethoxyphenyl)urea;
1-[1-[dideuterio(1H-pyrrolo[2,3-b]pyridin-4-yl)methyl]indol-4-yl]-3-(5-trifluoromethyl-2-methoxyphenyl)urea;
1-[1-[dideuterio(1H-pyrrolo[2,3-b]pyridin-4-yl)methyl]indol-4-yl]-3-(5-methyl-2-fluorophenyl)urea;
1-[1-[dideuterio(1H-pyrrolo[2,3-b]pyridin-4-yl)methyl]indol-4-yl]-3-(5-methoxyl-2-fluorophenyl)urea;
1-[1-[dideuterio(1H-pyrrolo[2,3-b]pyridin-4-yl)methyl]indol-4-yl]-3-(5-trifluoromethyl-2-fluorophenyl)urea;
1-[1-[dideuterio(1H-pyrrolo[2,3-b]pyridin-4-yl)methyl]indol-4-yl]-3-(5-chloro-2-fluorophenyl)urea;
1-[1-[dideuterio(1H-pyrrolo[2,3-b]pyridin-4-yl)methyl]indol-4-yl]-3-(5-bromo-2-fluorophenyl)urea; or
1-[1-[dideuterio(1H-pyrrolo[2,3-b]pyridin-4-yl)methyl]indol-4-yl]-3-(5-methyl-2-trifluoromethylphenyl)urea.

In some embodiments, the compound is:
1-[1-[dideuterio(1H-pyrrolo[2,3-b]pyridin-3-yl)methyl]indolin-4-yl]-3-phenylurea;
1-[1-[dideuterio(1H-pyrrolo[2,3-b]pyridin-3-yl)methyl]indolin-4-yl]-3-(2-fluorophenyl)urea;
1-[1-[dideuterio(1H-pyrrolo[2,3-b]pyridin-3-yl)methyl]indolin-4-yl]-3-(3-fluorophenyl)urea;
1-[1-[dideuterio(1H-pyrrolo[2,3-b]pyridin-3-yl)methyl]indolin-4-yl]-3-(4-fluorophenyl)urea;
1-[1-[dideuterio(1H-pyrrolo[2,3-b]pyridin-3-yl)methyl]indolin-4-yl]-3-(2-chlorophenyl)urea;
1-[1-[dideuterio(1H-pyrrolo[2,3-b]pyridin-3-yl)methyl]indolin-4-yl]-3-(3-chlorophenyl)urea;
1-[1-[dideuterio(1H-pyrrolo[2,3-b]pyridin-3-yl)methyl]indolin-4-yl]-3-(4-chlorophenyl)urea;
1-[1-[dideuterio(1H-pyrrolo[2,3-b]pyridin-3-yl)methyl]indolin-4-yl]-3-(2-bromophenyl)urea;
1-[1-[dideuterio(1H-pyrrolo[2,3-b]pyridin-3-yl)methyl]indolin-4-yl]-3-(3-bromophenyl)urea;
1-[1-[dideuterio(1H-pyrrolo[2,3-b]pyridin-3-yl)methyl]indolin-4-yl]-3-(4-bromophenyl)urea;
1-[1-[dideuterio(1H-pyrrolo[2,3-b]pyridin-3-yl)methyl]indolin-4-yl]-3-(2-methylphenyl)urea;
1-[1-[dideuterio(1H-pyrrolo[2,3-b]pyridin-3-yl)methyl]indolin-4-yl]-3-(3-methylphenyl)urea;
1-[1-[dideuterio(1H-pyrrolo[2,3-b]pyridin-3-yl)methyl]indolin-4-yl]-3-(4-methylphenyl)urea;
1-[1-[dideuterio(1H-pyrrolo[2,3-b]pyridin-3-yl)methyl]indolin-4-yl]-3-(2-methoxyphenyl)urea;
1-[1-[dideuterio(1H-pyrrolo[2,3-b]pyridin-3-yl)methyl]indolin-4-yl]-3-(3-methoxyphenyl)urea;
1-[1-[dideuterio(1H-pyrrolo[2,3-b]pyridin-3-yl)methyl]indolin-4-yl]-3-(4-methoxyphenyl)urea;
1-[1-[dideuterio(1H-pyrrolo[2,3-b]pyridin-3-yl)methyl]indolin-4-yl]-3-(2-trifluoromethoxyphenyl)urea;
1-[1-[dideuterio(1H-pyrrolo[2,3-b]pyridin-3-yl)methyl]indolin-4-yl]-3-(3-trifluoromethoxyphenyl)urea;
1-[1-[dideuterio(1H-pyrrolo[2,3-b]pyridin-3-yl)methyl]indolin-4-yl]-3-(4-trifluoromethoxyphenyl)urea;
1-[1-[dideuterio(1H-pyrrolo[2,3-b]pyridin-3-yl)methyl]indolin-4-yl]-3-(2-trifluoromethylphenyl)urea;
1-[1-[dideuterio(1H-pyrrolo[2,3-b]pyridin-3-yl)methyl]indolin-4-yl]-3-(3-trifluoromethylphenyl)urea;
1-[1-[dideuterio(1H-pyrrolo[2,3-b]pyridin-3-yl)methyl]indolin-4-yl]-3-(4-trifluoromethylphenyl)urea;
1-[1-[dideuterio(1H-pyrrolo[2,3-b]pyridin-3-yl)methyl]indolin-4-yl]-3-(2-cyanophenyl)urea;
1-[1-[dideuterio(1H-pyrrolo[2,3-b]pyridin-3-yl)methyl]indolin-4-yl]-3-(3-cyanophenyl)urea;
1-[1-[dideuterio(1H-pyrrolo[2,3-b]pyridin-3-yl)methyl]indolin-4-yl]-3-(4-cyanophenyl)urea;
1-[1-[dideuterio(1H-pyrrolo[2,3-b]pyridin-3-yl)methyl]indolin-4-yl]-3-(5-fluoro-2-methoxyphenyl)urea;
1-[1-[dideuterio(1H-pyrrolo[2,3-b]pyridin-3-yl)methyl]indolin-4-yl]-3-(5-chloro-2-methoxyphenyl)urea;
1-[1-[dideuterio(1H-pyrrolo[2,3-b]pyridin-3-yl)methyl]indolin-4-yl]-3-(5-bromo-2-methoxyphenyl)urea;
1-[1-[dideuterio(1H-pyrrolo[2,3-b]pyridin-3-yl)methyl]indolin-4-yl]-3-(5-methyl-2-methoxyphenyl)urea;
1-[1-[dideuterio(1H-pyrrolo[2,3-b]pyridin-3-yl)methyl]indolin-4-yl]-3-(2,5-dimethoxyphenyl)urea;
1-[1-[dideuterio(1H-pyrrolo[2,3-b]pyridin-3-yl)methyl]indolin-4-yl]-3-(5-trifluoromethyl-2-methoxyphenyl)urea;
1-[1-[dideuterio(1H-pyrrolo[2,3-b]pyridin-3-yl)methyl]indolin-4-yl]-3-(5-methyl-2-fluorophenyl)urea;
1-[1-[dideuterio(1H-pyrrolo[2,3-b]pyridin-3-yl)methyl]indolin-4-yl]-3-(5-methoxyl-2-fluorophenyl)urea;
1-[1-[dideuterio(1H-pyrrolo[2,3-b]pyridin-3-yl)methyl]indolin-4-yl]-3-(5-trifluoromethyl-2-fluorophenyl)urea;
1-[1-[dideuterio(1H-pyrrolo[2,3-b]pyridin-3-yl)methyl]indolin-4-yl]-3-(5-chloro-2-fluorophenyl)urea;
1-[1-[dideuterio(1H-pyrrolo[2,3-b]pyridin-3-yl)methyl]indolin-4-yl]-3-(5-bromo-2-fluorophenyl)urea; or
1-[1-[dideuterio(1H-pyrrolo[2,3-b]pyridin-3-yl)methyl]indolin-4-yl]-3-(5-methyl-2-trifluoromethylphenyl)urea.

In some embodiments, the compound is:
1-[1-[dideuterio(1H-pyrrolo[2,3-b]pyridin-4-yl)methyl]indolin-4-yl]-3-phenylurea;
1-[1-[dideuterio(1H-pyrrolo[2,3-b]pyridin-4-yl)methyl]indolin-4-yl]-3-(2-fluorophenyl)urea;
1-[1-[dideuterio(1H-pyrrolo[2,3-b]pyridin-4-yl)methyl]indolin-4-yl]-3-(3-fluorophenyl)urea;
1-[1-[dideuterio(1H-pyrrolo[2,3-b]pyridin-4-yl)methyl]indolin-4-yl]-3-(4-fluorophenyl)urea;
1-[1-[dideuterio(1H-pyrrolo[2,3-b]pyridin-4-yl)methyl]indolin-4-yl]-3-(2-chlorophenyl)urea;
1-[1-[dideuterio(1H-pyrrolo[2,3-b]pyridin-4-yl)methyl]indolin-4-yl]-3-(3-chlorophenyl)urea;
1-[1-[dideuterio(1H-pyrrolo[2,3-b]pyridin-4-yl)methyl]indolin-4-yl]-3-(4-chlorophenyl)urea;
1-[1-[dideuterio(1H-pyrrolo[2,3-b]pyridin-4-yl)methyl]indolin-4-yl]-3-(2-bromophenyl)urea;
1-[1-[dideuterio(1H-pyrrolo[2,3-b]pyridin-4-yl)methyl]indolin-4-yl]-3-(3-bromophenyl)urea;
1-[1-[dideuterio(1H-pyrrolo[2,3-b]pyridin-4-yl)methyl]indolin-4-yl]-3-(4-bromophenyl)urea;
1-[1-[dideuterio(1H-pyrrolo[2,3-b]pyridin-4-yl)methyl]indolin-4-yl]-3-(2-methylphenyl)urea;
1-[1-[dideuterio(1H-pyrrolo[2,3-b]pyridin-4-yl)methyl]indolin-4-yl]-3-(3-methylphenyl)urea;
1-[1-[dideuterio(1H-pyrrolo[2,3-b]pyridin-4-yl)methyl]indolin-4-yl]-3-(m-tolyl)urea hydrochloride;
1-[1-[dideuterio(1H-pyrrolo[2,3-b]pyridin-4-yl)methyl]indolin-4-yl]-3-(4-methylphenyl)urea;
1-[1-[dideuterio(1H-pyrrolo[2,3-b]pyridin-4-yl)methyl]indolin-4-yl]-3-(2-methoxyphenyl)urea;
1-[1-[dideuterio(1H-pyrrolo[2,3-b]pyridin-4-yl)methyl]indolin-4-yl]-3-(3-methoxyphenyl)urea;
1-[1-[dideuterio(1H-pyrrolo[2,3-b]pyridin-4-yl)methyl]indolin-4-yl]-3-(4-methoxyphenyl)urea;
1-[1-[dideuterio(1H-pyrrolo[2,3-b]pyridin-4-yl)methyl]indolin-4-yl]-3-(2-trifluoromethoxyphenyl)urea;
1-[1-[dideuterio(1H-pyrrolo[2,3-b]pyridin-4-yl)methyl]indolin-4-yl]-3-(3-trifluoromethoxyphenyl)urea;
1-[1-[dideuterio(1H-pyrrolo[2,3-b]pyridin-4-yl)methyl]indolin-4-yl]-3-(4-trifluoromethoxyphenyl)urea;
1-[1-[dideuterio(1H-pyrrolo[2,3-b]pyridin-4-yl)methyl]indolin-4-yl]-3-(2-trifluoromethylphenyl)urea;
1-[1-[dideuterio(1H-pyrrolo[2,3-b]pyridin-4-yl)methyl]indolin-4-yl]-3-(3-trifluoromethylphenyl)urea;
1-[1-[dideuterio(1H-pyrrolo[2,3-b]pyridin-4-yl)methyl]indolin-4-yl]-3-(4-trifluoromethylphenyl)urea;
1-[1-[dideuterio(1H-pyrrolo[2,3-b]pyridin-4-yl)methyl]indolin-4-yl]-3-(2-cyanophenyl)urea;
1-[1-[dideuterio(1H-pyrrolo[2,3-b]pyridin-4-yl)methyl]indolin-4-yl]-3-(3-cyanophenyl)urea;
1-[1-[dideuterio(1H-pyrrolo[2,3-b]pyridin-4-yl)methyl]indolin-4-yl]-3-(4-cyanophenyl)urea;
1-[1-[dideuterio(1H-pyrrolo[2,3-b]pyridin-4-yl)methyl]indolin-4-yl]-3-(5-fluoro-2-methoxyphenyl)urea;
1-[1-[dideuterio(1H-pyrrolo[2,3-b]pyridin-4-yl)methyl]indolin-4-yl]-3-(5-chloro-2-methoxyphenyl)urea;
1-[5-chloro-2-(trideuteriomethoxy)phenyl]-3-[1-[dideuterio(1H-pyrrolo[2,3-b]pyridin-4-yl)methyl]indolin-4-yl]urea hydrochloride;
1-(5-chloro-2-methoxyphenyl)-3-[1-[dideuterio(1H-pyrrolo[2,3-b]pyridin-4-yl)methyl]indolin-4-yl]urea hydrochloride;
1-[1-[dideuterio(1H-pyrrolo[2,3-b]pyridin-4-yl)methyl]indolin-4-yl]-3-(5-bromo-2-methoxyphenyl)urea;
1-[1-[dideuterio(1H-pyrrolo[2,3-b]pyridin-4-yl)methyl]indolin-4-yl]-3-(5-methyl-2-methoxyphenyl)urea;
1-[1-[dideuterio(1H-pyrrolo[2,3-b]pyridin-4-yl)methyl]indolin-4-yl]-3-(2,5-dimethoxyphenyl)urea;
1-[1-[dideuterio(1H-pyrrolo[2,3-b]pyridin-4-yl)methyl]indolin-4-yl]-3-(5-trifluoromethyl-2-methoxyphenyl)urea;
1-[1-[dideuterio(1H-pyrrolo[2,3-b]pyridin-4-yl)methyl]indolin-4-yl]-3-(5-methyl-2-fluorophenyl)urea;
1-[1-[dideuterio(1H-pyrrolo[2,3-b]pyridin-4-yl)methyl]indolin-4-yl]-3-(5-methoxyl-2-fluorophenyl)urea;
1-[1-[dideuterio(1H-pyrrolo[2,3-b]pyridin-4-yl)methyl]indolin-4-yl]-3-(5-trifluoromethyl-2-fluorophenyl)urea;
1-[1-[dideuterio(1H-pyrrolo[2,3-b]pyridin-4-yl)methyl]indolin-4-yl]-3-(5-chloro-2-fluorophenyl)urea;
1-(5-chloro-2-fluorophenyl)-3-[1-[dideuterio(1H-pyrrolo[2,3-b]pyridin-4-yl)methyl]indolin-4-yl]urea hydrochloride;
1-[1-[dideuterio(1H-pyrrolo[2,3-b]pyridin-4-yl)methyl]indolin-4-yl]-3-(5-bromo-2-fluorophenyl)urea; or
1-[1-[dideuterio(1H-pyrrolo[2,3-b]pyridin-3-yl)methyl]indolin-4-yl]-3-(5-methyl-2-trifluoromethylphenyl)urea.

In some embodiments, the compound is:
1-[1-[(2-amino-4-pyridyl)dideuteriomethyl]indol-4-yl]-3-phenylurea;
1-[1-[(2-amino-4-pyridyl)dideuteriomethyl]indol-4-yl]-3-(2-fluorophenyl)urea;
1-[1-[(2-amino-4-pyridyl)dideuteriomethyl]indol-4-yl]-3-(3-fluorophenyl)urea;
1-[1-[(2-amino-4-pyridyl)dideuteriomethyl]indol-4-yl]-3-(4-fluorophenyl)urea;
1-[1-[(2-amino-4-pyridyl)dideuteriomethyl]indol-4-yl]-3-(2-chlorophenyl)urea;
1-[1-[(2-amino-4-pyridyl)dideuteriomethyl]indol-4-yl]-3-(3-chlorophenyl)urea;
1-[1-[(2-amino-4-pyridyl)dideuteriomethyl]indol-4-yl]-3-(4-chlorophenyl)urea;
1-[1-[(2-amino-4-pyridyl)dideuteriomethyl]indol-4-yl]-3-(2-bromophenyl)urea;
1-[1-[(2-amino-4-pyridyl)dideuteriomethyl]indol-4-yl]-3-(3-bromophenyl)urea;
1-[1-[(2-amino-4-pyridyl)dideuteriomethyl]indol-4-yl]-3-(4-bromophenyl)urea;
1-[1-[(2-amino-4-pyridyl)dideuteriomethyl]indol-4-yl]-3-(2-methylphenyl)urea;
1-[1-[(2-amino-4-pyridyl)dideuteriomethyl]indol-4-yl]-3-(3-methylphenyl)urea;
1-[1-[(2-amino-4-pyridyl)dideuteriomethyl]indol-4-yl]-3-(4-methylphenyl)urea;
1-[1-[(2-amino-4-pyridyl)dideuteriomethyl]indol-4-yl]-3-(2-methoxyphenyl)urea;
1-[1-[(2-amino-4-pyridyl)dideuteriomethyl]indol-4-yl]-3-(3-methoxyphenyl)urea;
1-[1-[(2-amino-4-pyridyl)dideuteriomethyl]indol-4-yl]-3-(4-methoxyphenyl)urea;
1-[1-[(2-amino-4-pyridyl)dideuteriomethyl]indol-4-yl]-3-(2-trifluoromethoxyphenyl)urea;
1-[1-[(2-amino-4-pyridyl)dideuteriomethyl]indol-4-yl]-3-(3-trifluoromethoxyphenyl)urea;
1-[1-[(2-amino-4-pyridyl)dideuteriomethyl]indol-4-yl]-3-(4-trifluoromethoxyphenyl)urea;
1-[1-[(2-amino-4-pyridyl)dideuteriomethyl]indol-4-yl]-3-(2-trifluoromethylphenyl)urea;
1-[1-[(2-amino-4-pyridyl)dideuteriomethyl]indol-4-yl]-3-(3-trifluoromethylphenyl)urea;
1-[1-[(2-amino-4-pyridyl)dideuteriomethyl]indol-4-yl]-3-(4-trifluoromethylphenyl)urea;
1-[1-[(2-amino-4-pyridyl)dideuteriomethyl]indol-4-yl]-3-(2-cyanophenyl)urea;
1-[1-[(2-amino-4-pyridyl)dideuteriomethyl]indol-4-yl]-3-(3-cyanophenyl)urea;
1-[1-[(2-amino-4-pyridyl)dideuteriomethyl]indol-4-yl]-3-(4-cyanophenyl)urea;
1-[1-[(2-amino-4-pyridyl)dideuteriomethyl]indol-4-yl]-3-(5-fluoro-2-methoxyphenyl)urea;
1-[1-[(2-amino-4-pyridyl)dideuteriomethyl]indol-4-yl]-3-(5-chloro-2-methoxyphenyl)urea;
1-[1-[(2-amino-4-pyridyl)-dideuteriomethyl]indol-4-yl]-3-(5-chloro-2-methoxyphenyl)urea hydrochloride;
1-[1-[(2-amino-4-pyridyl)dideuteriomethyl]indol-4-yl]-3-(5-bromo-2-methoxyphenyl)urea;
1-[1-[(2-amino-4-pyridyl)dideuteriomethyl]indol-4-yl]-3-(5-methyl-2-methoxyphenyl)urea;
1-[1-[(2-amino-4-pyridyl)dideuteriomethyl]indol-4-yl]-3-(2,5-dimethoxyphenyl)urea;
1-[1-[(2-amino-4-pyridyl)dideuteriomethyl]indol-4-yl]-3-(5-trifluoromethyl-2-methoxyphenyl)urea;
1-[1-[(2-amino-4-pyridyl)dideuteriomethyl]indol-4-yl]-3-(5-methyl-2-fluorophenyl)urea;
1-[1-[(2-amino-4-pyridyl)dideuteriomethyl]indol-4-yl]-3-(5-methoxyl-2-fluorophenyl)urea;
1-[1-[(2-amino-4-pyridyl)dideuteriomethyl]indol-4-yl]-3-(5-trifluoromethyl-2-fluorophenyl)urea;
1-[1-[(2-amino-4-pyridyl)dideuteriomethyl]indol-4-yl]-3-(5-chloro-2-fluorophenyl)urea;
1-[1-[(2-amino-4-pyridyl)dideuteriomethyl]indol-4-yl]-3-(5-bromo-2-fluorophenyl)urea; or
1-[1-[(2-amino-4-pyridyl)dideuteriomethyl]indol-4-yl]-3-(5-methyl-2-trifluoromethylphenyl)urea.

In some embodiments, the compound is:
1-[1-[dideuterio(1H-pyrrolo[2,3-b]pyridin-3-yl)methyl]indol-4-yl]-3-[2-(trideuteriomethoxy)phenyl]urea;
1-[1-[dideuterio(1H-pyrrolo[2,3-b]pyridin-3-yl)methyl]indol-4-yl]-3-[3-(trideuteriomethoxy)phenyl]urea;
1-[1-[dideuterio(1H-pyrrolo[2,3-b]pyridin-3-yl)methyl]indol-4-yl]-3-[4-(trideuteriomethoxy)phenyl]urea;
1-[1-[dideuterio(1H-pyrrolo[2,3-b]pyridin-3-yl)methyl]indol-4-yl]-3-[5-fluoro-2-(trideuteriomethoxy)phenyl]urea;
1-[1-[dideuterio(1H-pyrrolo[2,3-b]pyridin-3-yl)methyl]indol-4-yl]-3-[5-chloro-2-(trideuteriomethoxy)phenyl]urea;
1-[1-[dideuterio(1H-pyrrolo[2,3-b]pyridin-3-yl)methyl]indol-4-yl]-3-[5-bromo-2-(trideuteriomethoxy)phenyl]urea;
1-[1-[dideuterio(1H-pyrrolo[2,3-b]pyridin-3-yl)methyl]indol-4-yl]-3-[5-methyl-2-(trideuteriomethoxy)phenyl]urea;
1-[1-[dideuterio(1H-pyrrolo[2,3-b]pyridin-3-yl)methyl]indol-4-yl]-3-[2,5-di(trideuteriomethoxy)phenyl]urea;
1-[1-[dideuterio(1H-pyrrolo[2,3-b]pyridin-3-yl)methyl]indol-4-yl]-3-[5-trifluoromethyl-2-(trideuteriomethoxy)phenyl]urea; or
1-[1-[dideuterio(1H-pyrrolo[2,3-b]pyridin-3-yl)methyl]indol-4-yl]-3-[2-fluoro-5-(trideuteriomethoxy)phenyl]urea.

In some embodiments, the compound is:
1-(5-chloro-2-fluorophenyl)-3-[1-[dideuterio(1H-pyrrolo[2,3-b]pyridin-4-yl)methyl]indol-4-yl]urea hydrochloride;
1-[1-[dideuterio(1H-pyrrolo[2,3-b]pyridin-4-yl)methyl]indol-4-yl]-3-(m-tolyl)urea hydrochloride;
1-[1-[dideuterio(1H-pyrrolo[2,3-b]pyridin-4-yl)methyl]indol-4-yl]-3-[2-(trideuteriomethoxy)phenyl]urea;
1-[1-[dideuterio(1H-pyrrolo[2,3-b]pyridin-4-yl)methyl]indol-4-yl]-3-[3-(trideuteriomethoxy)phenyl]urea;
1-[1-[dideuterio(1H-pyrrolo[2,3-b]pyridin-4-yl)methyl]indol-4-yl]-3-[4-(trideuteriomethoxy)phenyl]urea;
1-[1-[dideuterio(1H-pyrrolo[2,3-b]pyridin-4-yl)methyl]indol-4-yl]-3-[5-fluoro-2-(trideuteriomethoxy)phenyl]urea
1-[1-[dideuterio(1H-pyrrolo[2,3-b]pyridin-4-yl)methyl]indol-4-yl]-3-[5-chloro-2-(trideuteriomethoxy)phenyl]urea;
1-(5-chloro-2-methoxyphenyl)-3-[1-[dideuterio(1H-pyrrolo[2,3-b]pyridin-4-yl)methyl]indol-4-yl]urea hydrochloride;
1-[1-[dideuterio(1H-pyrrolo[2,3-b]pyridin-4-yl)methyl]indol-4-yl]-3-[5-bromo-2-(trideuteriomethoxy)phenyl]urea;
1-[1-[dideuterio(1H-pyrrolo[2,3-b]pyridin-4-yl)methyl]indol-4-yl]-3-[5-methyl-2-(trideuteriomethoxy)phenyl]urea;
1-[1-[dideuterio(1H-pyrrolo[2,3-b]pyridin-4-yl)methyl]indol-4-yl]-3-[2,5-di(trideuteriomethoxy)phenyl]urea;
1-[1-[dideuterio(1H-pyrrolo[2,3-b]pyridin-4-yl)methyl]indol-4-yl]-3-[5-trifluoromethyl-2-(trideuteriomethoxy)phenyl]urea; or
1-[1-[dideuterio(1H-pyrrolo[2,3-b]pyridin-4-yl)methyl]indol-4-yl]-3-[2-fluoro-5-(trideuteriomethoxy)phenyl]urea.

In some embodiments, the compound is:
1-[1-[(2-amino-4-pyridyl)dideuteriomethyl]indol-4-yl]-3-[2-(trideuteriomethoxy)phenyl]urea;
1-[1-[(2-amino-4-pyridyl)dideuteriomethyl]indol-4-yl]-3-[3-(trideuteriomethoxy)phenyl]urea;
1-[1-[(2-amino-4-pyridyl)dideuteriomethyl]indol-4-yl]-3-[4-(trideuteriomethoxy)phenyl]urea;
1-[1-[(2-amino-4-pyridyl)dideuteriomethyl]indol-4-yl]-3-[5-fluoro-2-(trideuteriomethoxy)phenyl]urea;
1-[1-[(2-amino-4-pyridyl)dideuteriomethyl]indol-4-yl]-3-[5-chloro-2-(trideuteriomethoxy)phenyl]urea;
1-[1-[(2-amino-4-pyridyl)dideuteriomethyl]indol-4-yl]-3-[5-bromo-2-(trideuteriomethoxy)phenyl]urea;
1-[1-[(2-amino-4-pyridyl)dideuteriomethyl]indol-4-yl]-3-[5-methyl-2-(trideuteriomethoxy)phenyl]urea;
1-[1-[(2-amino-4-pyridyl)dideuteriomethyl]indol-4-yl]-3-[2,5-di(trideuteriomethoxy)phenyl]urea;
1-[1-[(2-amino-4-pyridyl)dideuteriomethyl]indol-4-yl]-3-[5-trifluoromethyl-2-(trideuteriomethoxy)phenyl]urea; or
1-[1-[(2-amino-4-pyridyl)dideuteriomethyl]indol-4-yl]-3-[2-fluoro-5-(trideuteriomethoxy)phenyl]urea.

In some other embodiments, the compound is:
1-[1-[(2-amino-4-pyridyl)-dideuteriomethyl]indolin-4-yl]-3-(5-chloro-2-methoxyphenyl)urea; or
1-[1-[(2-amino-4-pyridyl)-dideuteriomethyl]indolin-4-yl]-3-[5-chloro-2-(trideuteriomethoxy)phenyl]urea.

In some other embodiments, the compound is:
1-[1-[(2-amino-4-pyridyl)methyl]indolin-4-yl]-3-[5-chloro-2-(trideuteriomethoxy)phenyl]urea; or
1-[1-[(2-amino-4-pyridyl)methyl]indol-4-yl]-3-[5-chloro-2-(trideuteriomethoxy)phenyl]urea.

In some other embodiments, the compound is:
1-[5-chloro-2-(trideuteriomethoxy)phenyl]-3-[1-[deuterio(1H-pyrrolo[2,3-b]pyridin-3-yl)methyl]indolin-4-yl]urea;
1-(5-chloro-2-methoxyphenyl)-3-[1-[deuterio(1H-pyrrolo[2,3-b]pyridin-3-yl)methyl]indolin-4-yl]urea;
1-(5-chloro-2-fluorophenyl)-3-[1-[deuterio(1H-pyrrolo[2,3-b]pyridin-3-yl)methyl]indolin-4-yl]urea; or
1-[1-[deuterio(1H-pyrrolo[2,3-b]pyridin-3-yl)methyl]indolin-4-yl]-3-(m-tolyl)urea.

In some other embodiments, the compound is:
1-[5-chloro-2-(trideuteriomethoxy)phenyl]-3-[1-[deuterio(1H-pyrrolo[2,3-b]pyridin-3-yl)methyl]indol-4-yl]urea;
1-(5-chloro-2-methoxyphenyl)-3-[1-[deuterio(1H-pyrrolo[2,3-b]pyridin-3-yl)methyl]indol-4-yl]urea;
1-(5-chloro-2-fluorophenyl)-3-[1-[deuterio(1H-pyrrolo[2,3-b]pyridin-3-yl)methyl]indol-4-yl]urea; or
1-[1-[deuterio(1H-pyrrolo[2,3-b]pyridin-3-yl)methyl]indol-4-yl]-3-(m-tolyl)urea.

In some other embodiments, the compound is:
1-[1-[(2-amino-4-pyridyl)-dideuteriomethyl]indol-4-yl]-3-(5-chloro-2-methoxyphenyl)urea hydrochloride;
1-[1-[(2-amino-4-pyridyl)-dideuteriomethyl]indol-4-yl]-3-[5-chloro-2-(trideuteriomethoxy)phenyl]urea;
1-[5-chloro-2-(trideuteriomethoxy)phenyl]-3-[1-[dideuterio(1H-pyrrolo[2,3-b]pyridin-4-yl)methyl]indolin-4-yl]urea hydrochloride;
1-[5-chloro-2-(trideuteriomethoxy)phenyl]-3-[1-[dideuterio(1H-pyrrolo[2,3-b]pyridin-4-yl)methyl]indol-4-yl]urea hydrochloride;
1-(5-chloro-2-methoxyphenyl)-3-[1-[dideuterio(1H-pyrrolo[2,3-b]pyridin-4-yl)methyl]indolin-4-yl]urea hydrochloride;
1-(5-chloro-2-methoxyphenyl)-3-[1-[dideuterio(1H-pyrrolo[2,3-b]pyridin-4-yl)methyl]indol-4-yl]urea hydrochloride;
1-(5-chloro-2-fluorophenyl)-3-[1-[dideuterio(1H-pyrrolo[2,3-b]pyridin-4-yl)methyl]indolin-4-yl]urea hydrochloride;
1-(5-chloro-2-fluorophenyl)-3-[1-[dideuterio(1H-pyrrolo[2,3-b]pyridin-4-yl)methyl]indol-4-yl]urea hydrochloride;
1-[1-[dideuterio(1H-pyrrolo[2,3-b]pyridin-4-yl)methyl]indolin-4-yl]-3-(m-tolyl)urea hydrochloride; or
1-[1-[dideuterio(1H-pyrrolo[2,3-b]pyridin-4-yl)methyl]indol-4-yl]-3-(m-tolyl)urea hydrochloride.

Another aspect of the present invention provides pharmaceutical compositions each including a therapeutically effective amount of any of the compounds described above and a pharmaceutically acceptable carrier. The pharmaceutical compositions can be in any form that is suitable for an intended administration method, e.g., injectable, aerosol, cream, gel, capsule, pill, tablet, syrup, eye drop, or ointment.

The compounds described above exhibit inhibitory effect on one or more protein kinases, e.g., VEGFR2, PDGFRβ, c-Met or EphB4.

The compounds described above exhibit, in majority of cases (e.g., at least 50%, at least 75%, at least 90%), higher metabolic stability against degradation by liver enzymes of organisms (e.g., mouse and rat) than their deuterium non-enriched counterparts.

Accordingly, another aspect of the present invention provides a method for treating a patient having a condition mediated by an abnormal protein kinase activity (e.g., overexpressed protein kinase). The method includes administering to the patient in need thereof a therapeutically effective amount of any of the compounds or pharmaceutical compositions described above. Each of the suitable compounds or pharmaceutical compositions can be administered in a suitable manner, e.g., intravenously, subcutaneously, orally, parenterally, or topically. Examples of such a protein kinase include VEGFR2, c-Met, RON, PDGFRα, PDGFRβ, EphB4, Alk, Tie-1, Tie-2, Flt3, FGFR1, FGFR2, FGFR3, FGFR4, EGFR, Her2, Abl, Aurora A, Aurora B, Aurora C, Src, Lck, IGF-1R, or IR. Examples of such a condition include cancer, tumor, rheumatoid arthritis, autoimmune disease, acute inflammation, nephritis, diabetic retinitis, psoriasis, or macular degeneration. The tumor or cancer can be, e.g., bone cancer (e.g., Ewing's sarcoma, osteosarcoma, chondrosarcoma, or orthopaedics links), brain and CNS tumor (e.g., acoustic neuroma, spinal cord tumor, brain tumor ring of hope), breast cancer, breast cancer, colorectal cancer (e.g., anal cancer), endocrine cancer (e.g., adrenocortical carcinoma, pancreatic cancer(e.g. pancreatic carcinoma such as exocrine pancreatic carcinoma), pituitary cancer, thyroid cancer, parathyroid cancer, thymus cancer, multiple endocrine neoplasia, or other endocrine cancer), gastrointestinal cancer (e.g., stomach cancer, esophageal cancer, small intestine cancer, gall bladder cancer, liver cancer, extra-hepatic bile duct cancer, or gastrointestinal carcinoid tumor), genitourinary cancer (e.g., testicular cancer, penile cancer, or prostate cancer), gynecological cancer (e.g., cervical cancer, ovarian cancer, vaginal cancer, uterus/endometrium cancer, vulva cancer, gestational trophoblastic cancer, fallopian tube cancer, or uterine sarcoma), head and neck cancer (e.g., oral cavity, lip, salivary gland cancer, larynx, hypopharynx, oropharynx cancer, nasal, paranasal, or nasopharynx cancer), leukemia (e.g., acute lymphocytic leukemia, acute myeloid leukemia, chronic lymphocytic leukemia, chronic myeloid leukemia, hairy cell leukemia, acute promyelocytic leukemia, plasma cell leukemia), lung cancer (e.g., adenocarcinoma, small cell lung cancer, or non-small cell lung cancer), lymphoma (e.g., Hodgkin's Disease, Non-Hodgkin's Lymphoma, AIDS-related Lymphoma), eye cancer (e.g., retinoblastoma or intraocular melanoma), skin cancer (e.g., melanoma, non-melanoma skin cancer or Merkel cell cancer), soft tissue sarcoma (e.g., Kaposi's Sarcoma), urinary system cancer (e.g., kidney cancer, Wilm's tumor, bladder cancer, urethral cancer, or transitional cell cancer), and other types or related disorders (e.g., histiocytosis, mesothelioma, metastatic cancer, carcinoid tumors, neurofibromatosis, germ cell tumors, desmoplasic small round cell tumor, malignant rhabdoid tumor, desmoid tumor, ataxia-telangiectasia, Nijmegen breakage syndrome, Rothmund-Thomson syndrome, Li-Fraumeni Syndrome, von Hipple-Lindau Disease, Beckwith-Wiedemann syndrome, Down's syndrome, Denys-Drash syndrome, WAGR syndrome, or CIN cervical intraepithelial neoplasm).

Chemical entities of the present invention include, but are not limited to compounds of Formula I and all pharmaceutically acceptable forms thereof. Pharmaceutically acceptable forms of the compounds recited herein include pharmaceutically acceptable salts, solvates, crystal forms (including polymorphs and clathrates), chelates, non-covalent complexes, prodrugs, and mixtures thereof. In certain embodiments, the compounds described herein are in the form of pharmaceutically acceptable salts. Hence, the terms “chemical entity” and “chemical entities” also encompass pharmaceutically acceptable salts, solvates, crystal forms, chelates, non-covalent complexes, prodrugs, and mixtures.

As noted above, prodrugs also fall within the scope of chemical entities, for example ester or amide derivatives of the compounds of Formula I. The term “prodrugs” includes any compounds that become compounds of Formula I when or after administered to a patient, e.g., upon metabolic processing of the prodrug. Examples of prodrugs include, but are not limited to, acetate, formate, and benzoate and like derivatives of functional groups (such as alcohol or amine groups) in the compounds of Formula I.

As used herein, the term “solvate” refers to the chemical entity formed by the interaction of a solvent and a compound. Suitable solvates are pharmaceutically acceptable solvates, such as hydrates, including monohydrates and hemi-hydrates.

As used herein, the term “chelate” refers to the chemical entity formed by the coordination of a compound to a metal ion at two (or more) points.

As used herein, the term “non-covalent complex” refers to the chemical entity formed by the interaction of a compound and another molecule wherein a covalent bond is not formed between the compound and the molecule. For example, complexation can occur through van der Waals interactions, hydrogen bonding, and electrostatic interactions (also called ionic bonding).

The term “active agent” is used to indicate a chemical entity which has biological activity. In certain embodiments, an “active agent” is a compound having pharmaceutical utility. For example an active agent may be an anti-cancer therapeutic.

As used herein, the term “alkyl”, used alone or as part of a larger moiety (e.g., as in “cycloalkenylalkyl” or “haloalkyloxy”), refers to a saturated aliphatic hydrocarbon group. It can contain 1 to 8 (e.g., 1 to 6 or 1 to 4) carbon atoms. As a moiety, it can be denoted as —CₙH₂ₙ₊₁. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, and 2-ethylhexyl. An alkyl group can be substituted (i.e., optionally substituted) with one or more substituents.

As used herein, the term “alkylene”, used alone or as part of a larger moiety (e.g., as in “arylalkyleneoxy” or “arylhaloalkylenroxy”), refers to a saturated aliphatic hydrocarbon group with two radical points for forming two covalent bonds with two other moieties. It can contain 1 to 8 (e.g., 1 to 6 or 1 to 4) carbon atoms. As a moiety, it can be denoted as —CₙH₂ₙ—. Examples of an alkylene group include, but are not limited to, methylene (—CH₂—), ethylene (—CH₂CH₂—), and propylene (—CH₂CH₂CH₂—).

As used herein, the term “alkynyl”, used alone or as part of a larger moiety (e.g., as in “alkynylalkyl” or “haloalkynylalkoxy”), refers to an aliphatic carbon group with at least one trip bond. It can contain 2 to 8 (e.g., 2 to 6 or 2 to 4) carbon atoms. An alkynyl group can be straight or branched. Examples of an alkynyl group include, but are not limited to, propargyl and butyryl.

As used herein, the term “alkenyl”, used alone or as part of a larger moiety (e.g., as in “alkenylalkyl” or “alkenylalkoxy”), refers to an aliphatic carbon group with at least one double bond. It can contain 2 to 8 (e.g., 2 to 6 or 2 to 4) carbon atoms. An alkenyl group with one double bond can be denoted as —CₙH₂ₙ₋₁, or —CₙH₂ₙ₋₃ with two double bonds. Like an alkyl group, an alkenyl group can be straight or branched. Examples of an alkenyl group include, but are not limited to, allyl, isoprenyl, 2-butenyl, and 2-hexenyl.

As used herein, the term “cycloalkyl”, used alone or as part of a larger moiety (e.g., as in “cycloalkylalkyl” or “halocycloalkylalkoxy”), refers to a saturated carbocyclic mono-, bi-, or tri-cyclic (fused or bridged or spiral) ring system. It can contain 3 to 12 (e.g., 3 to 10, or 5 to 10) carbon atoms. Examples of cycloalkyl groups include, but are not limited to, cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, azacycloalkyl, or ((aminocarbonyl)cycloalkyl)cycloalkyl.

As used herein, the term “cycloalkenyl”, used alone or as part of a larger moiety (e.g., as in “cycloalkenylalkyl” or “cyanocycloalkenylalkoxy”), refers to a non-aromatic carbocyclic ring system having one or more double bonds. It can contain 3 to 12 (e.g., 3 to 10, or 5 to 10) carbon atoms. Examples of cycloalkenyl groups include, but are not limited to, cyclopentenyl, 1,4-cyclohexa-di-enyl, cycloheptenyl, cyclooctenyl, hexahydro-indenyl, octahydro-naphthyl, cyclohexenyl, cyclopentenyl, bicyclo[2.2.2]octenyl, or bicyclo[3.3.1]nonenyl.

As used herein, the term “heterocycloalkyl”, used alone or as part of a larger moiety (e.g., as in “heterocycloalkylalkyl” or “heterocycloalkoxy”), refers to a 3- to 16-membered mono-, bi-, or tri-cyclic (fused or bridged or spiral)) saturated ring structure, in which one or more of the ring atoms is a heteroatom (e.g., N, O, S, or combinations thereof). In addition to the heteroatom(s), the heterocycloalkyl can contain 3 to 15 carbon atoms (e.g., 3 to 12 or 5 to 10). Examples of a heterocycloalkyl group include, but are not limited to, piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl. A monocyclic heterocycloalkyl group can be fused with a phenyl moiety such as tetrahydroisoquinoline.

As used herein, the term “aryl”, used alone or as part of a larger moiety (e.g., as in “aralkyl”, “aralkoxy,” or “haloaryloxyalkyl”), refers to a monocyclic (e.g., phenyl); bicyclic (e.g., indenyl, naphthalenyl, tetrahydronaphthyl, benzimidazole, benzothiazole, or tetrahydroindenyl); and tricyclic (e.g., fluorenyl, tetrahydrofluorenyl, tetrahydroanthracenyl, or anthracenyl) ring system in which the monocyclic ring system is aromatic (e.g., phenyl) or at least one of the rings in a bicyclic or tricyclic ring system is aromatic (e.g., phenyl). The bicyclic and tricyclic groups include benzo-fused 2- or 3-membered carbocyclic rings. For instance, a benzo-fused group includes phenyl fused with two or more C₄₋₈ carbocyclic moieties.

As used herein, the term “heteroaryl” refers to a monocyclic, bicyclic, or tricyclic ring system having 5 to 15 ring atoms wherein at least one of the ring atoms is a heteroatom (e.g., N, O, S, or combinations thereof) and in which the monocyclic ring system is aromatic or at least one of the rings in the bicyclic or tricyclic ring systems is aromatic. It can contain e.g., 5 to 12 or 8 to 10 carbon atoms. A heteroaryl group includes a benzo-fused ring system having 2 to 3 rings. For example, a benzo-fused group includes benzo fused with one or two 4- to 8-membered heterocycloalkyl moieties (e.g., indolizyl, indolyl, isoindolyl, 3H-indolyl, indolinyl, benzo[b]furyl, benzo[b]thiophenyl, quinolinyl, or isoquinolinyl). Some examples of heteroaryl are pyridyl, 1H-indazolyl, furyl, pyrrolyl, thienyl, thiazolyl, oxazolyl, imidazolyl, tetrazolyl, benzofuryl, isoquinolinyl, benzithiazolyl, xanthenyl, thioxanthenyl, phenothiazinyl, dihydroindolyl, benzo[1,3]dioxolyl, benzo[b]furyl, benzo[b]thiophenyl, indazolyl, benzimidazolyl, benzthiazolyl, puryl, quinolinyl, quinazolinyl, phthalazyl, quinazolyl, quinoxalyl, isoquinolinyl, 4H-quinolizyl, benzo-1,2,5-thiadiazolyl, and 1,8-naphthyridyl.

As used herein, the term “bridged bicyclic ring system” refers to a bicyclic heterocycloalkyl ring system or bicyclic cycloalkyl ring system in which the rings have at least two common atoms. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxabicyclo[2.2.2]octyl, 1-azabicyclo[2.2.2]octyl, 3-azabicyclo[3.2.1]octyl, and 2,6-dioxatricyclo[3.3.1.03,7]nonyl.

As used herein, the term “halo” refers to fluoro, chloro, bromo, or iodo.

As used herein, the term “independent”, e.g., as in “optionally substituted with one or more independent R³ groups”, means that when the number of substituent is more than one (e.g., two or three), these multiple substituents can be the same or different.

As used herein, the term “optionally”, e.g., as in “optionally substituted with”, means that the moiety at issue is either substituted or not substituted, and that the substitution occurs only when chemically feasible. For instance, H cannot be substituted with a substituent; and a covalent bond or —C(═O)— group cannot be substituted with a substituent.

As used herein, an “oxo” group refers to ═O.

As used herein, a “carbonyl” group refers to —C(O)— or —C(═O)—.

As used herein, a “cyano” group refers to —CN.

As used herein, a “urea” group refers to the structure —NR_{X}—CO—NR_{Y}R_{Z} when terminally included in a compound or —NR_{X}—CO—NR_{Y}— when internally included in a compound.

As used herein, the term “substituted,” whether preceded by the term “optionally” or not, refers to the replacement of hydrogen radicals in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this invention are those combinations that result in the formation of stable or chemically feasible compounds.

As used herein, the term “stable” or “chemically feasible” refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

As used herein, the term “or” as in, e.g., “one or more independent halo, alkoxy, alkyl . . . cyano, oxo, or optionally substituted heterocycloalkyl” (emphasis added) can mean “or” or “and.” In other words, under this scenario, for instance, the substituents (when more than one) can be two halo groups or one halo and one alkyl. In another example, “VEGFR2 or c-Met” can mean “VEGFR2,” “c-Met,” or “VEGFR2 and c-Met.”

As used herein, the phrase “pharmaceutically acceptable salt(s)” means those salts of the compounds of the invention that are safe and effective for internal use (or topical use, if needed) in a subject (e.g., a mammal such as a human patient, a dog, or a cat) and that possess the desired biological activity. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts. For a review on pharmaceutically acceptable salts, see, e.g., Berge et al., J. Pharm. Sci., 1977, 66, 1-19, the contents of which are incorporated herein by reference.

As used herein, a “subject” for treatment generally refers to and thus may be interchangeable with a “patient,” such as an animal (e.g., a mammal such as a human, a cat, or a dog).

As used herein, an “effective amount” is defined as the amount required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, N.Y., 537 (1970).

Unless otherwise specified, all cyclic radical moieties identified herein can be bonded to another moiety in any of the formulae included herein at any of its ring atoms.

Unless otherwise stated, the structures depicted herein are meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C— or ¹⁴C-enriched carbon are within the scope of this invention. Such compounds are useful, for example, as analytical tools or probes in biological assays.

As used herein, the term “therapeutically effective amount” of a compound or pharmaceutical composition of this invention refers to an amount effective, when administered to a human or non-human patient, for the treatment of a disease. For example, a therapeutically effective amount may be an amount sufficient to treat a disease or disorder responsive to kinase inhibition. The therapeutically effective amount may be ascertained experimentally, e.g., by assaying blood concentration of the chemical entity, or theoretically, by calculating bioavailability.

As used herein, the term “significant” refers to any detectable change that is statistically significant in a standard parametric or nonparametric test of hypothesis such as Student's T-test, where p<0.05.

As used herein, the term “patient” or “subject” refers to an animal, such as a mammal, for example a human, a dog, or a cat, that has been or will be the object of treatment, observation or experiment. The methods of the invention can be useful in both human therapy and veterinary applications.

As used herein, the term “angiogenesis kinase” refers to a kinase involved in angiogenesis. Its examples include VEGFR2, PDGFRβ, EphB4 and c-Met.

As used herein, the term “inhibition” refers to a decrease in kinase activity as a direct or indirect response to the presence of compounds of Formula I, relative to the activity of the kinase in the absence of the compound. The decrease may be due to the direct interaction of the compound with the kinase, or due to the interaction of the compound with one or more other factors that in turn affect kinase activity. For example, the presence of the compound may, for example, decrease kinase activity by directly binding to the kinase, by causing (directly or indirectly) another factor to decrease the kinase activity, or by (directly or indirectly) decreasing the amount of kinase present in the cell or organism.

As used here in, the term “treatment” or “treating” refers to any treatment of a disease in a patient, including: (a) preventing the disease, that is, causing the clinical symptoms of the disease not to develop; (b) inhibiting the disease; (c) slowing or arresting the development of clinical symptoms; or (d) relieving the disease, that is, causing the regression of clinical symptoms.

As used herein, the term “diseases or disorders responsive to kinase inhibition” refer to pathologic conditions that depend, at least in part, on the activity of one or more protein kinases, for example, angiogenesis kinases. Kinases either directly or indirectly participate in the signal transduction pathways of a variety of cellular activities including cell proliferation, differentiation, and invasion. Diseases responsive to kinase inhibition include but are not limited to tumor growth, angiogenesis supporting solid tumor growth, and diseases characterized by excessive growth of local vasculature such as diabetic retinopathy, macular degeneration, and inflammation.

As used herein, the term “change in angiogenesis” refers to a change in the vascular network or quality of vasculature. Change in angiogenesis may be measured by many parameters and, for instance, may be assessed by delayed appearance of neovascular structures, slowed development of neovascular structures, decreased occurrence of neovascular structures, changes in vascular permeability, changes in blood flow, slowed or decreased severity of angiogenesis-dependent disease effects, arrested vasculature growth, or regression of previous vasculature.

### DETAILED DESCRIPTION OF THE INVENTION

The present invention provides compounds of Formula I, or a pharmaceutically acceptable salt thereof.

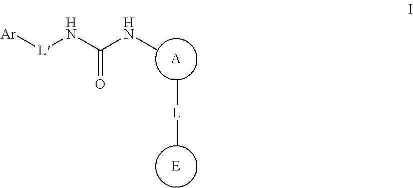

### In Formula I:

E is E1, E2 or E3:

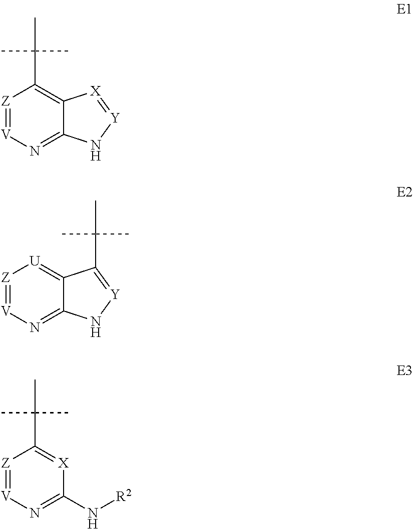

U, V, X, Y, and Z are each independently N or C—R¹;

L is a deuterium-enriched C₁₋₈alkylene, which is optionally substituted with one or more independent R³;

R² is H, C₁₋₈alkyl, C₆₋₁₂aryl, C₅₋₁₂heteroaryl, —C(═O)—C₁₋₈alkyl, —C(═O)—C₆₋₁₂aryl, or —C(═O)—C₅₋₁₂heteroaryl, each of which is optionally substituted with one or more independent Q¹;

L′ is a covalent bond, —C(═O)—, —C(═O)—C₁₋₈alkylene, or C₁₋₈alkylene, each of which is optionally substituted with one or more independent R⁴;

A is A1, A2, or A3:

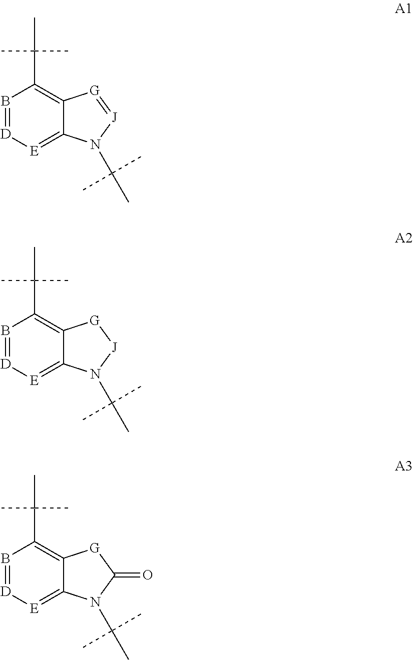

wherein B, D, E, G, and J are each independently N or CH;

each of A1, A2, and A3 is optionally substituted with one or more independent R⁵;

Ar is aryl or heteroaryl, each of which is optionally substituted with one or more independent R⁶;

R¹, R³, R⁴, R⁵, and R⁶ are each independently H, halo, —CN, —CF₃, —NO₂, —NH₂, —OH, —OCF₃, —OCH₃, —CO₂H, C₁₋₈alkyl, C₂₋₈alkenyl, C₂₋₈alkynyl, C₃₋₁₂cycloalkyl, C₃₋₁₂heterocycloalkyl, C₆₋₁₂ arylC₁₋₈alkyl, or C₅₋₁₂heteroarylC₁₋₈alkyl, each of which is optionally substituted with one or more independent Q²;

or R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q²;

Q¹ and Q² are each independently H, halo, —CN, —CF₃, —OCF₃, —NO₂, oxo, C₁₋₈alkyl, C₂₋₈alkenyl, C₂₋₈alkynyl, C₃₋₁₂ cycloalkyl, C₃₋₁₂heterocycloalkyl, C₆₋₁₂aryl, C₅₋₁₂heteroaryl, C₈₋₁₂heterocycloaryl, —OR⁷, —S(O)ₙR⁸, —NR⁹R¹⁰, —SO₂NR⁹R¹⁰, —C(O)R¹¹, —C(O)NR⁹R¹⁰, —C(O)OR⁷, —OC(O)R¹¹, —NR⁹C(O)R¹¹, —NR⁹S(O)₂R¹², —NR¹³C(O)NR⁹R¹⁰, —NR¹³S(O)₂NR⁹R¹⁰, or —NR¹³S(O)NR⁹R¹⁰, each of which is optionally substituted with one or more independent H, halo, —CN, —OH, —NH₂, —NO₂, oxo, —CF₃, —OCF₃, —CO₂H, —S(O)ₙH, C₁₋₈alkyl, C₆₋₁₂ aryl, C₅₋₁₂heteroaryl, C₃₋₁₂cycloalkyl, C₃₋₁₂heterocycloalkyl, C₈₋₁₂heterocycloaryl, or —O—C₁₋₈alkyl, each of which is optionally partially or fully halogenated;

R⁷, R⁸, R⁹, R¹⁰, R¹¹, R¹², and R¹³ are each independently H, C₁₋₈alkyl, C₂₋₈alkenyl, C₂₋₈alkynyl, C₃₋₁₂ cycloalkyl, C₃₋₁₂heterocycloalkyl, C₆₋₁₂aryl, C₅₋₁₂heteroaryl, or C₈₋₁₂heterocycloaryl;

or when in —NR⁹R¹⁰, R⁹ and R¹⁰, together with the nitrogen atom to which they are attached, form a 3- to 12-membered saturated or unsaturated ring, wherein the ring optionally includes one or more heteroatoms each independently being O, N, or S(O)ₙ; and n is 0, 1, or 2.

### General Synthetic Schemes

Compounds of this invention may be synthesized from commercially available or known starting materials by known methods. Exemplary synthetic routes to produce these compounds are provided in the schemes shown below wherein the substituents are as defined herein unless otherwise noted. These generic schemes are for illustration only and not limiting, and they can be applied to preparation of other compounds that include different variables than those explicitly shown below.

The following abbreviations are used:
NMR=Nuclear magnetic resonance
TMS=Tetramethylsilane
DCM=Dichloromethane
THF=Tetrahydrofuran
EtOAc=Ethyl acetate
MeCN=Acetonitrile
DMSO=Dimethylsulfoxide
Boc=t-Butyloxycarbonyl
DMF=N,N-Dimethylformamide
DME=Dimethyl ether
TFA=Trifluoroacetic acid
CDCl₃=Deuterated chloroform
DMSO-d₆=Deuterated dimethylsulfoxide
TLC=Thin layer chromatography
HPLC=High performance liquid chromatography
Min=Minute(s)
h=Hour(s)
d=Day(s)
RT or rt=Room temperature
t_{R}=Retention time
L=Liter
mL=Milliliter
mmol or mM=Millimole
g=Gram
mg=Milligram
LG=Leaving Group
PG=Protecting Group
BFEE=BF₃.OEt₂
DMAP=4-Dimethylaminopyridine
Ts or TS or p-Ts=p-Toluenesulfonyl
(Boc)₂O=Di-tert-butyl dicarbonate
DDQ=2,3-Dichloro-5,6-dicyano-1,4-benzoquinone

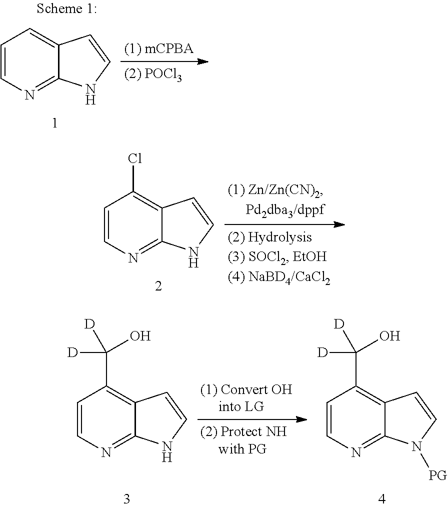

Intermediate 4 can be prepared from commercially available 7-azaindole (1) according to Scheme 1 illustrated above. Specifically, 4-chloro-7-azaindole (2) can be obtained by oxidation of starting material 1 with an oxidant, such as m-chloroperoxybenzoic acid (mCPBA) or hydrogen peroxide or other peroxyacid, followed by treatment with POCl₃ or SOCl₂ to give compound 2. Compound 2 can then be converted into alcohol 3 by Pd-catalyzed cyanation, base- or acid-mediated hydrolysis, esterification, and NaBD₄ or LiAlD₄ reduction. The hydroxyl group in compound 3 is then replaced with a leaving group (LG), such as Cl, Br, I, MeSO₃—, TfO—, and TsO—, by reacting with SOCl₂, CBr₄+PPh₃, PBr₃, MeSO₂Cl, Tf₂O, TsCl, etc. The NH group on the azaindolyl ring can be protected by a commonly used nitrogen protecting group (PG), e.g., Boc or Cbz or other carbamate, PhSO₂— or other organosulfonyl, p-methoxybenzyl (PMB), methoxymethyl (MOM), [β-(trimethylsilyl)ethoxy]methyl (SEM), etc.

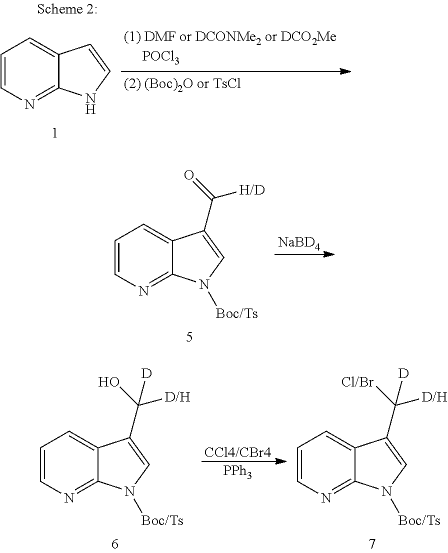

Intermediates 7 can be prepared from commercially available 7-azaindole (1) as shown in Scheme 2. On treatment with DMF or DCONMe₂ or DCO₂Me and POCl₃, compound 1 is converted to an aldehyde, which is then protected by a Boc or Ts group to give compound 5. Compound 5 can then be reduced to a primary alcohol 6 with NaBD₄. Treatment of alcohol 6 with carbon tetrachloride/triphenylphosphine or carbon tetrabromide/triphenylphosphine affords corresponding chloride or bromide compound 7.

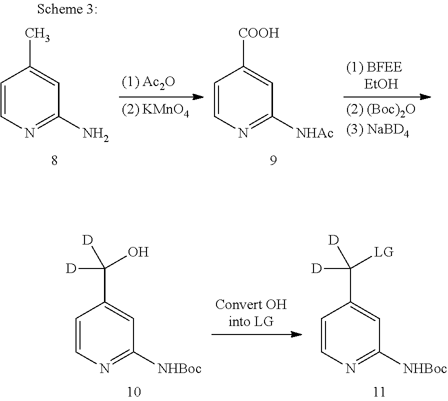

Intermediates 11 can be prepared from commercially available 2-amino-4-methylpyridine (8) as shown in Scheme 3. Protection of the amino group of compound 8 with acetyl followed by methyl oxidation provides an acid 9, which can then be converted into ester, de-acylated (by BF₃.OEt₂), and re-protected with Boc, and reduced to alcohol 10 with NaBD₄. The hydroxyl group in compound 10 can be replaced with a leaving group (LG), such as Cl, Br, I, MeSO₃—, TfO—, or TsO—, by reacting with SOCl₂, CBr₄+PPh₃, PBr₃, MeSO₂Cl, Tf₂O, or TsCl.

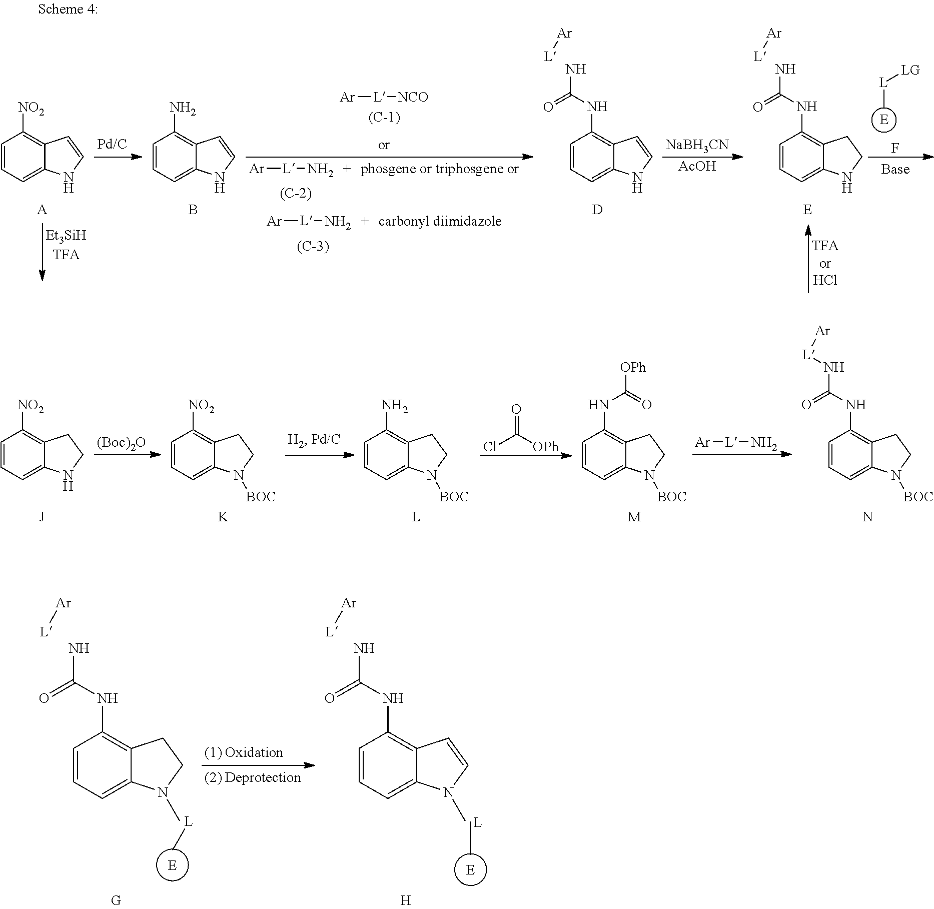

Scheme 4 shows one exemplary method for synthesizing compounds of Formula I. In this method, commercially available 4-nitroindole (A) is first reduced to 4-aminoindole (B), which then reacts with isocyanate (C-1) or amine (C-2) mediated by phosgene or triphosgene or carbonyl diimidazole to afford a urea product (D). Upon treatment with NaBH₃CN+AcOH, compound D can be converted into compound E. Alkylation of compound E with intermediate F (prepared by methods described in Schemes 1-3) gives compound G, which can be subsequently converted to product H after removal of protecting group (PG) under acidic or basic conditions. Intermediate E can also be prepared by following A→J→K→L→M→N→E synthetic sequence. 4-Nitroindole (A) is reduced into J, followed by Boc protection of NH and NO₂ reduction to afford L. Compound L is converted into carbamate M, which is subsequently converted into urea N by reacting with amine Ar-L′—NH₂. Removal of Boc by TFA or HCl gives E.

### General Experimental and Analytical Methods

Unless otherwise noted, all materials/reagents were obtained from commercial suppliers and used without further purification. NMR spectra were recorded on a Bruker or Varian 300 or 400 MHz instrument at ambient temperature with TMS or the residual solvent peak as the internal standard. The line positions or multiples are given in ppm (δ) and the coupling constants (J) are given as absolute values in Hertz (Hz). The multiplicities in ¹H NMR spectra are abbreviated as follows: s (singlet), d (doublet), t (triplet), q (quartet), quint (quintet), m (multiplet), m_{c} (centered multiplet), br or broad (broadened). Mass spectra (MS) were measured by ESI methods. Reactions were monitored by thin layer chromatography (TLC) on silica gel 60 F-254 (0.2 mm) and visualized using UV light. Flash chromatography was performed with silica gel (400-230 mesh).

### Preparation I: 2-Methoxy-5-chlorophenyl isocyanate (Int-4)

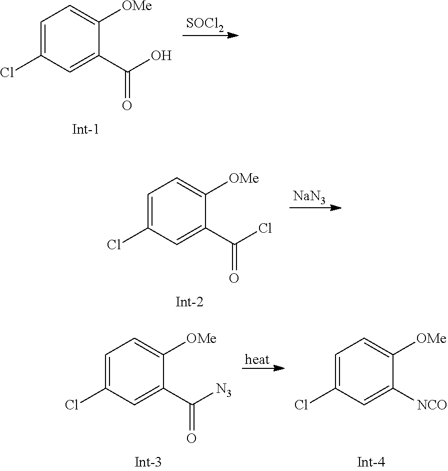

To a suspension of 2-methoxy-5-chlorobenzoic acid (Int-1, 18.7 g, 100 mmol) in dry dichloromethane (120 mL) under nitrogen was added dropwise thionyl chloride (25 mL, 130 mmol). After the reaction mixture was refluxed for 2 hours, a clear solution was formed. Evaporation of excess thionyl chloride and solvent gave a solid product Int-2, which was used directly in the next step without purification.

To a solution of acid chloride Int-2 in acetone (100 mL) under nitrogen was added sodium azide (7.8 g, 120 mmol) and 50 mL water. After being stirred for 2 hours at room temperature, additional 50 mL water was added. The mixture was filtered and the filter cake was washed with water and dried to afford acyl azide Int-3 as white solid, which was used directly in the next step without purification.

The acyl azide Int-3 obtained from previous step was dissolved in dry toluene (100 mL). The resulted solution was added slowly to an empty round-bottom flask preheated in an oil bath at 110° C. Evolution of nitrogen was observed, which ceased after about 1 h of addition. The reaction mixture was then cooled to room temperature and solvent was removed under reduced pressure to yield a yellowish solid residue, which was recrystallized from petroleum ether to afford the desired product Int-4 as white crystals (10.3 g, 56% for 3 steps), which turn yellowish on standing.

MS (ESI⁺): m/z 238.0 [M+MeOH+Na, ³⁵Cl]⁺, 240.0 [M+MeOH+Na, ³⁷Cl]⁺.

### Preparation II: tert-Butyl 4-bromodideuteriomethylpyridin-2-ylcarbamate (Int-11)

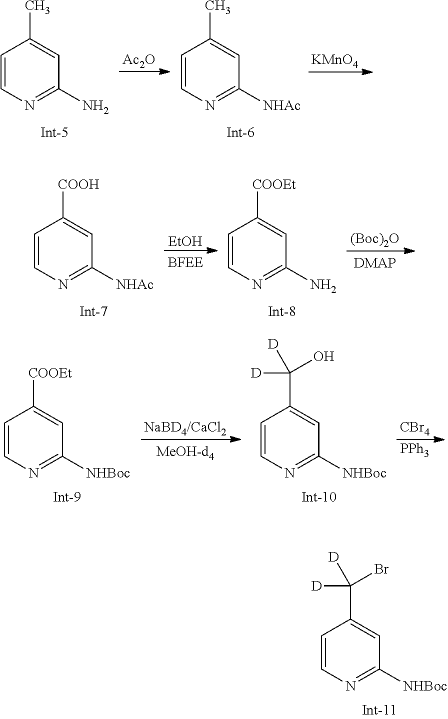

A round-bottom flask was charged with Int-5 (10.8 g, 0.1 mol), AcOH (10 mL), and acetic anhydride (12 mL). The mixture was brought to reflux until the reaction was complete (as determined by TLC), and then poured into ice-water, basified to pH 9 with ammonium hydroxide, and extracted with ethyl acetate. The organic layer was washed with water, brine, dried over anhydrous sodium sulfate, filtered, and concentrated under reduced pressure to dryness to give Int-6 (13.5 g, 90%).

Int-6 (10 g, 67 mmol) was added to a solution of potassium permanganate (15 g) in water (60 mL), and the mixture was heated to 60° C., then another batch of potassium permanganate (34 g) was added to the mixture in portions. After addition, the temperature was brought to 90° C. and stirred until the reaction finished (by TLC). The mixture was filtered while hot, the filtrate was cooled to room temperature, neutralized and acidified with concentrated hydrochloric acid. The solids were collected by filtration to afford Int-7 (4.2 g, 35%), which was used directly in the next step without purification.

BF₃.OEt₂ (BFEE, 7.3 mL, 46 mmol) was added dropwise to a solution of Int-7 (3.6 g, 20 mmol) in 50 mL of anhydrous ethanol, the mixture was then heated to reflux overnight. After the reaction was complete (as determined by TLC), 80 mL of a 10% NaHCO₃ solution was added, and the resulting mixture was extracted with ethyl acetate. The organic layer was washed with water, brine, dried over anhydrous sodium sulfate, filtered, and concentrated under reduced pressure to dryness to give Int-8 (2.5 g, 75%).

A round-bottom flask was charged with Int-8 (2.4 g, 14.5 mmol), (Boc)₂O (4.3 g, 20 mmol), and tert-butanol (30 mL). The mixture was stirred at 60° C. for 16 hours, then concentrated to dryness, and the residue was purified by flash column chromatography to provide Int-9 (3.4 g, 88%).

¹H NMR (400 MHz, DMSO-d₆) δ 10.07 (s, 1H), 8.42 (dd, J=5.1, 0.8 Hz, 1H), 8.31 (s, 1H), 7.44 (dd, J=5.1, 1.4 Hz, 1H), 4.35 (q, J=7.1 Hz, 2H), 1.48 (s, 9H), 1.32 (t, J=7.1 Hz, 3H).

To a solution of Int-9 (533 mg, 2 mmol) in methanol-d₄ (5 mL) was added anhydrous calcium chloride (330 mg, 3 mmol). The mixture was cooled to 0° C., sodium borodeuteride (420 mg, 10 mmol) was added in one portion after 10 minutes. The resulting mixture was stirred for 5 min at 0° C. and then 4 h at room temperature. Water was added, the mixture was extracted with EtOAc. The combined organic phases were washed with brine, dried over anhydrous sodium sulfate, filtered, concentrated under reduced pressure to provide the product Int-10.

¹H NMR (400 MHz, DMSO-d₆) δ 9.66 (s, 1H), 8.14 (dd, J=5.1, 0.5 Hz, 1H), 7.80 (s, 1H), 6.94 (dd, J=5.1, 1.4 Hz, 1H), 5.36 (s, 1H), 1.47 (s, 9H).

To a solution of Int-10 (450 mg, 2 mmol) in DCM (10 mL) was added triphenylphosphine (630 mg, 2.4 mmol). The mixture was cooled to 0° C., a solution of carbon tetrabromide (800 mg, 2.4 mmol) in DCM (5 mL) was added dropwise. The resulting mixture was stirred for 5 min at 0° C. and then 4 h at room temperature, then concentrated to dryness, the residue was purified by flash column chromatography to provide Int-11 (450 mg, 78% yield).

¹H NMR (400 MHz, CDCl₃) δ 8.17 (d, J=4.8 Hz, 1H), 8.01 (s, 1H), 7.93 (s, 1H), 6.92 (dd, J=5.2, 1.5 Hz, 1H), 1.47 (s, 9H).

### Preparation III: tert-Butyl 4-[chloro(dideuterio)methyl]pyrrolo[2,3-b]pyridine-1-carboxylate (Int-15)

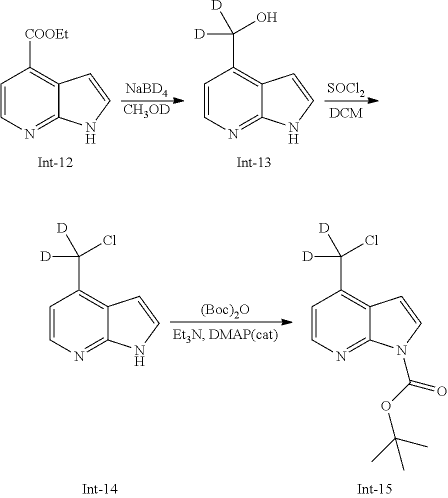

To a solution of ethyl 1H-pyrrolo[2,3-b]pyridine-4-carboxylate (Int-12, prepared according to WO2011/023081, 950 mg, 5 mmol) in methanol-d₁ (10 mL) was added sodium borodeuteride (1.05 g, 25 mmol) in portions. The resulting mixture was stirred at room temperature for 2 h and then refluxing overnight. The reaction mixture was concentrated, and to the residue was added water (50 mL), the mixture was extracted with EtOAc/MeOH (10:1, 3×50 mL). The combined organic phases were washed with brine, dried over anhydrous sodium sulfate, filtered, concentrated under reduced pressure to provide the product Int-13 (0.75 g, yield: 100%), which was used directly in the next step without purification.

To a solution of Int-13 (750 mg, 5 mmol) in DCM (10 mL) was added thionyl chloride (1.2 g, 10 mmol). The resulting mixture was stirred at room temperature for 1 hour, and then concentrated. To the residue was added water (10 mL) and EtOAc (50 mL). The mixture was washed with sat. NaHCO₃ (aq), extracted with EtOAc (25 mL). The combined organic phases were washed with brine, dried over anhydrous sodium sulfate, filtered, concentrated under reduced pressure to afford the product Int-14 (0.76 g, yield: 91%), which was used directly in the next step without purification.

To a solution of Int-14 (670 mg, 4 mmol), Et₃N (1.17 g, 11.6 mmol), and catalytical amount of DMAP in DCM (10 mL) was added (Boc)₂O (0.96 g, 4.4 mmol). The resulting mixture was stirred at room temperature for 30 minutes. The reaction mixture was concentrated and the residue was purified by column chromatography to afford the desired product Int-15 (0.5 g, yield: 47%).

¹H NMR (400 MHz, DMSO-d₆) δ 8.38 (d, J=4.9 Hz, 1H), 7.83 (s, 1H), 7.33 (d, J=4.9 Hz, 1H), 6.87 (d, J=4.1 Hz, 1H), 1.59 (s, 9H).

### Preparation IV: 3-[Bromo(deuterio)methyl]-1-(p-tolylsulfonyl)pyrrolo[2,3-b]pyridine (Int-19)

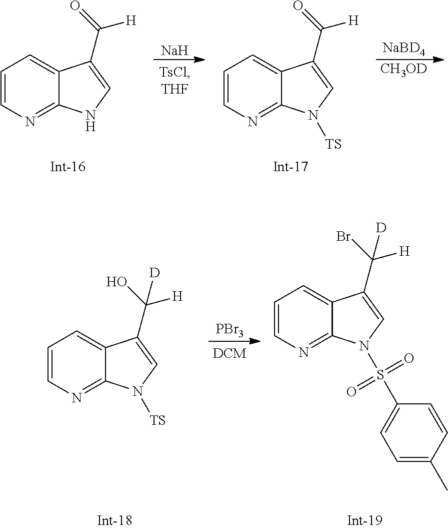

At 0° C., to a solution of Int-16 (4.5 g, 30.8 mmol) in dry THF (50 mL) was added sodium hydride (2.0 g, 61 mmol) in portions. The resulting mixture was stirred at the same temperature for 30 minutes, p-TsCl (7.05 g, 36.9 mmol) was then added. The reaction was allowed to warm to room temperature over 1 h under stirring and quenched with ice water (50 mL). The mixture was extracted with EtOAc (3×50 mL). The combined organic phases were washed with brine, dried over anhydrous sodium sulfate, filtered, concentrated under reduced pressure to give the crude product, which was trituated with petroleum ether/EtOAc (10:1, 30 mL) to afford Int-17 as a light yellow solid (5.4 g, yield: 58%).

¹H NMR (400 MHz, CDCl₃) δ 9.97 (s, 1H), 8.44 (t, J=5.7 Hz, 2H), 8.32 (s, 1H), 8.09 (d, J=8.3 Hz, 2H), 7.25 (m, 3H), 2.33 (s, 3H).

To a solution of Int-17 (5.4 g, 18.03 mmol) in methanol-d₁ (20 mL) was added sodium borodeuteride (0.9 g, 21.6 mmol) in portions. The resulting mixture was stirred at room temperature for 30 minutes and then at 60° C. for 1 hour. The reaction mixture was concentrated, and to the residue was added ice water (50 mL), the mixture was extracted with EtOAc (3×50 mL). The combined organic phases were washed with brine, dried over anhydrous sodium sulfate, filtered, concentrated under reduced pressure to give the crude product, which was trituated with EtOAc (10 mL) to afford Int-18 as a light yellow solid (4.0 g, yield: 73%), which was used directly in the next step without purification.

To a solution of Int-18 (4.0 g, 13 mmol) in DCM (50 mL) was added PBr₃ (7.18 g, 26 mmol) in portions. The resulting mixture was stirred at room temperature for 30 minutes, and then poured into ice water (50 mL). The pH of the mixture was adjusted to 7-8 using sat. NaHCO₃ (aq). Layers were separated and the aqueous layer was extracted with DCM (25 mL). The combined organic phases were washed with brine, dried over anhydrous sodium sulfate, filtered, concentrated under reduced pressure, the residue was purified by column chromatography (petroleum ether/EtOAc: 10:1) to afford the desired product Int-19 (3.2 g, yield: 67%) as a white solid.

¹H NMR (400 MHz, CDCl₃) δ 8.39 (d, J=4.7 Hz, 1H), 8.02 (d, J=8.2 Hz, 2H), 7.91 (d, J=7.8 Hz, 1H), 7.72 (s, 1H), 7.26-7.13 (m, 3H), 4.53 (s, 1H), 2.31 (s, 3H).

### Preparation V: tent-Butyl 4-(phenoxycarbonylamino)indoline-1-carboxylate (Int-24)

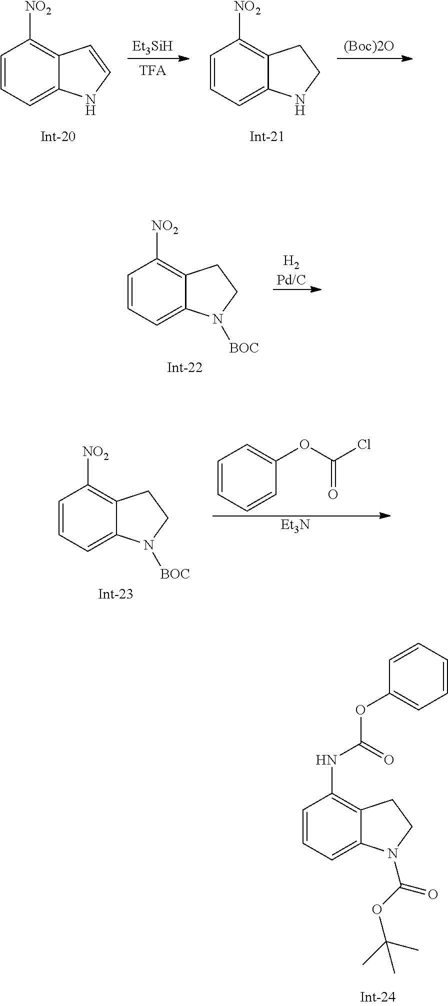

To a suspension of Int-20 (16.2 g, 100 mmol) in TFA (150 mL) was added Et₃SiH (30.2 g, 260 mmol). The resulting mixture was stirred at 50° C. for 1 hour. The solvent was removed under reduced pressure and the residue was dissolved in DCM (100 mL). Sat. NaHCO₃ (aq) was added cautiously to adjust to neutral pH. The organic phase was collected and concentrated to dryness. The residue was trituated with petroleum ether (25 mL) to afford Int-21 (16 g, yield: 97%).

¹H NMR (400 MHz, CDCl₃) δ 7.47 (d, J=8.3 Hz, 1H), 7.13 (t, J=8.0 Hz, 1H), 6.82 (d, J=7.7 Hz, 1H), 4.05 (s, 1H), 3.68 (t, J=8.5 Hz, 2H), 3.54 (t, J=8.4 Hz, 2H).

To a solution of Int-21 (16 g, 97.5 mmol), Et₃N (10.9 g, 107.3 mmol), and catalytical amount of DMAP in DCM (75 mL) was added (Boc)₂O (23.4 g, 107.3 mmol). The resulting mixture was stirred at room temperature for 2 days. The reaction mixture was diluted with DCM (150 mL) and water (150 mL). Layers were separated and organic layer was dried over anhydrous sodium sulfate, filtered, concentrated under reduced pressure to give the crude product, which was trituated with EtOH (50 mL) to give the desired product Int-22 (19.6 g, yield: 75%).

¹H NMR (400 MHz, DMSO-d₆) δ 8.17-7.84 (m, 1H), 7.71 (dd, J=8.3, 0.9 Hz, 1H), 7.43 (t, J=8.2 Hz, 1H), 3.98 (t, J=8.7 Hz, 2H), 3.48 (t, J=8.7 Hz, 2H), 1.51 (s, 9H).

A mixture of Int-22 (18.6 g, 70.4 mmol) and 10% Pd/C (2 g) in MeOH (450 mL) was hydrogenated under 1 atmosphere hydrogen pressure for 24 hours. The catalyst was filtered off, filtrate was concentrated to give product Int-23 as an oil (16.5 g, yield: 100%).

¹H NMR (400 MHz, DMSO-d₆) δ 7.20-7.13 (m, 1H), 6.82 (t, J=7.9 Hz, 1H), 6.22 (d, J=8.8 Hz, 1H), 4.91 (s, br, 2H), 3.92-3.80 (m, 2H), 2.78 (t, J=6.9 Hz, 2H), 1.48 (s, 9H).

To a solution of phenyl chloroformate (13.4 g, 85.2 mmol) in DCM (30 mL) was added a solution of Int-23(13.3 g, 56.8 mmol) and Et₃N (6.9 g, 68.2 mmol) in DCM (70 mL) over a period of 3.5 hours. After the reaction was complete (indicated by TLC), the reaction mixture was washed with water (50 mL) and sat. NaHCO₃ (aq), dried over anhydrous sodium sulfate, filtered, concentrated under reduced pressure to give the crude product, which was purified with column chromatography to give the desired product Int-24(16 g, yield: 80%).

### Preparation VI: 5-Chloro-2-(trideuteriomethoxy)aniline (Int-27)

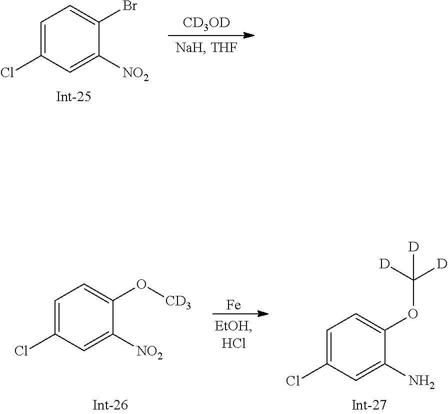

At 0° C., to a solution of CD₃OD (2.5 g, 70.5 mmol) in dry THF (150 mL) was added NaH (60% in mineral oil, 2.0 g, 51.7 mmol) in portions. After addition, the resulting mixture was stirred at 0° C. for 30 minutes. 1-Bromo-4-chloro-2-nitrobenzene (Int-25, 11.3 g, 46.3 mmol) was added in portions and the reaction was allowed to warm to room temperature. Stirring was continued ate room temperature for 5 hours. The reaction mixture was filtered, filtrate was concentrated to afford the crude product, which was trituated with petroleum ether/EtOAc (20:1) under reflux, cooled to room temperature to give Int-26 (7.2 g, yield: 81%) as a reddish solid.

To a refluxing mixture of Int-26 (6.7 g, 35 mmol) and iron powder (9.8 g, 175 mmol) in EtOH (70 mL) was added conc. HCl (1 mL) cautiously dropwise. TLC showed the reaction was complete in 4 hours. The reaction mixture was filtered, filtrate was concentrated and residue was purified by column chromatography to afford the desired product Int-27 (5.0 g, yield: 88%).

¹H NMR (400 MHz, CDCl₃) δ 6.63 (q, J=1.2 Hz, 1H), 6.60 (t, J=0.8 Hz, 2H), 3.34 (s, 2H).

### Preparation VII: 1-(5-Chloro-2-methoxyphenyl)-3-indolin-4-yl-urea (Int-30)

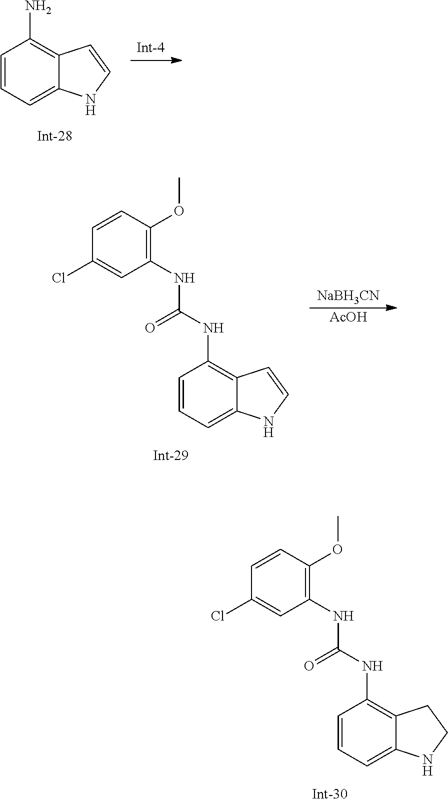

A round-bottom flask was charged with 4-nitroindole (4.8 g, 30 mmol), Palladium on carbon (10%, 480 mg), and THF (50 mL), and the mixture was stirred under one hydrogen atmosphere overnight. TLC showed the reaction was complete. The catalyst was filtered off and the filtrate was concentrated to afford 4-amino-1H-indole (Q-1), which was used directly in the next step without purification.

A solution of 5-chloro-2-methoxyphenyl isocyanate (Int-4, 5.5 g, 30 mmol) in DCM (20 mL) was added dropwise to the 4-amino-1H-indole in DCM (30 mL), and the resulting mixture was stirred overnight, then filtered to give the crude product 1-(5-chloro-2-methoxyphenyl)-3-(1H-indol-4-yl)urea (Int-29, 6.6 g, 70%), which was used directly in the next step without purification.

To a mixture of 1-(5-chloro-2-methoxyphenyl)-3-(1H-indol-4-yl)urea (Int-29, 6.3 g, 20 mmol) in acetic acid (50 mL) was added portionwise NaBH₃CN (1.9 g, 30 mmol), and the resulting mixture was stirred at room temperature for 1 hour. The solvent was evaporated and residue was diluted with water, neutralized with NaHCO₃ and extracted with EtOAc. The combined organic phases were washed with brine, dried and evaporated to give crude 1-(5-chloro-2-methoxyphenyl)-3-indolin-4-yl-urea (Int-30, 6.3 g, 100%), which was used directly in the next step without purification.

¹H NMR (300 MHz, DMSO-d₆) δ 8.72 (s, 1H), 8.65 (s, 1H), 8.22 (d, J=1.8 Hz, 1H), 7.14 (d, J=6.3 Hz, 1H), 7.03-6.94 (m, 2H), 6.83 (t, J=6.0 Hz, 1H), 6.20 (d, J=5.7 Hz, 1H), 5.47 (s, 1H), 3.88 (s, 3H), 3.44-3.40 (m, 2H), 2.86 (t, J=6.3 Hz, 2H).

### Preparation VIII: 1-(5-Chloro-2-methoxyphenyl)-3-indolin-4-yl-urea trifluoroacetate (Int-30.TFA)

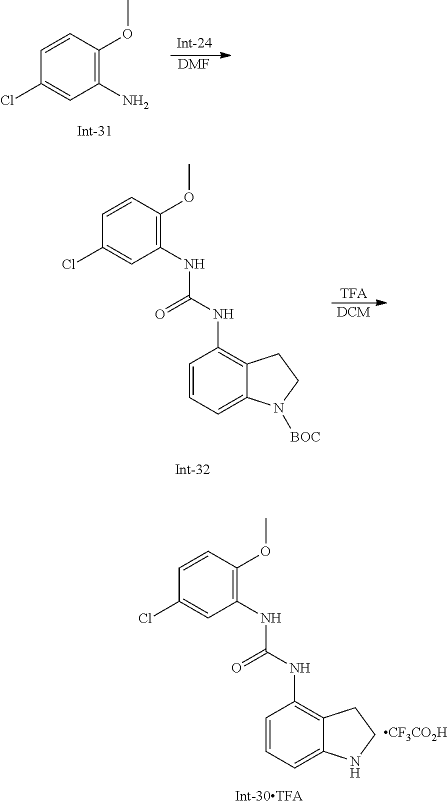

A solution of 5-chloro-2-methoxyaniline (Int-31, 0.64 g, 4 mmol) and Int-24 (1.42 g, 4.0 mmol) in DMF (10 mL) was stirred at 100° C. for 2 hours. After cooled down to room temperature, solvent was removed under reduced pressure, and the residue was purified by column chromatography to afford Int-32 (1.0 g, yield: 60%) as a white solid.

¹H NMR (300 MHz, CDCl₃) δ 7.80-6.85 (m, 7H), 6.57 (s, 1H), 3.97 (t, J=8.7 Hz, 2H), 2.98 (t, J=8.7 Hz, 2H), 1.51 (d, J=9.4 Hz, 9H).

To a solution of Int-32 (1.0 g, 2.4 mmol) in DCM (50 mL) was added TFA (10 equiv). The resulting mixture was stirred at room temperature for 2 days. Solvent was removed under reduced pressure to give Int-30.TFA (1.0 g, yield: 100%), which was used directly in the next step without purification.

### Preparation IX: 1-[5-Chloro-2-(trideuteriomethoxy)phenyl]-3-indolin-4-yl-urea trifluoroacetate (Int-34.TFA)

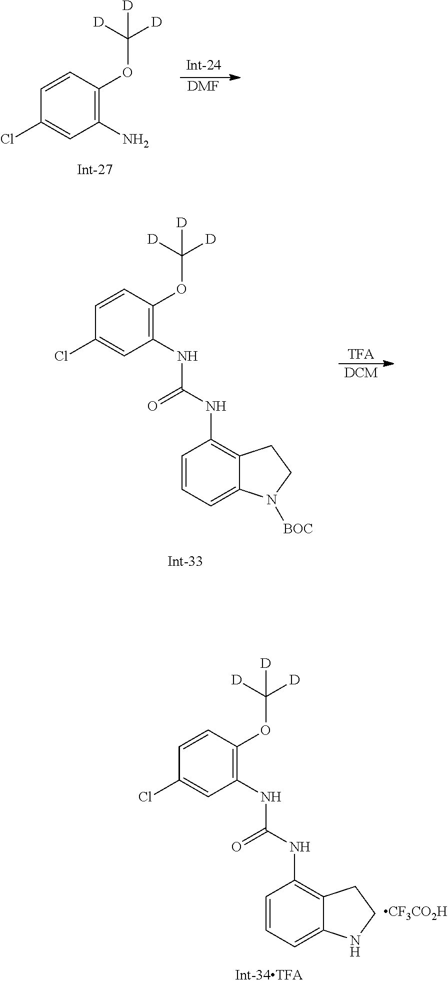

Int-34.TFA was prepared by following the same procedures described in Preparation XIII starting from Int-27 and Int-24.

### Examples 1 and 2

### 1-[1-[(2-Amino-4-pyridyl)-dideuteriomethyl]indolin-4-yl]-3-(5-chloro-2-methoxyphenyl)urea (Q-4 or Example 1) and 1-[1-[(2-amino-4-pyridyl)-dideuteriomethyl]indol-4-yl]-3-(5-chloro-2-methoxyphenyl)urea hydrochloride (Q-3 or Example 2)

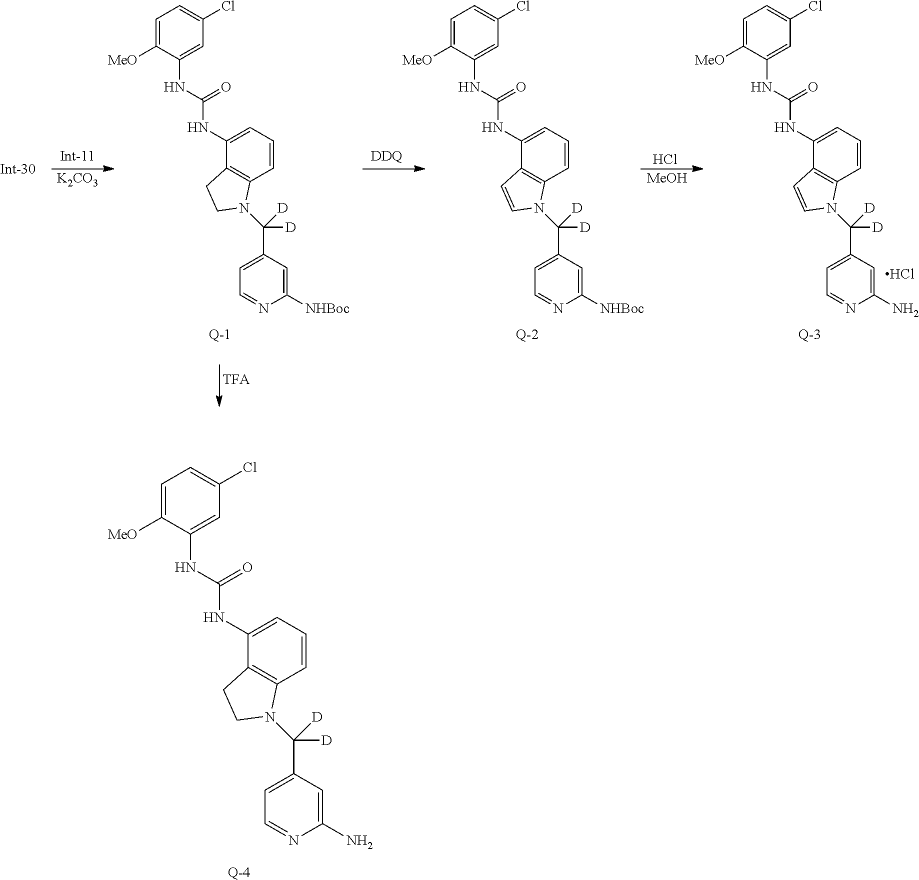

To a mixture of Int-30 (445 mg, 1.4 mmol), potassium carbonate (230 mg, 1.65 mmol), and DMF (10 mL) was added Int-11 (430 mg, 1.5 mmol) in portions. The mixture was stirred at 80° C. until the reaction was complete (as determined by TLC). Water was then added, the mixture was extracted with EtOAc. The combined organic phases were washed with brine, dried over anhydrous sodium sulfate, filtered, concentrated under reduced pressure, and purified by flash column chromatography to provide the desired product Q-1 (500 mg, 68%).

¹H NMR (400 MHz, CDCl₃) δ 8.49 (d, J=4.8 Hz, 1H), 8.29 (d, J=2.4 Hz, 1H), 7.65 (d, J=4.0 Hz, 1H), 7.43 (s, 1H), 7.22 (d, J=5.2 Hz, 1H), 7.12 (t, J=8.0 Hz, 2H), 6.91 (dd, J=8.8, 2.4 Hz, 1H), 6.86 (d, J=8.4 Hz, 1H), 6.74 (d, J=8.8 Hz, 1H), 6.64 (d, J=4.0 Hz, 1H), 6.36 (d, J=8.0 Hz, 1H), 6.32 (s, 1H), 4.51 (s, 2H), 3.78 (s, 3H), 3.40 (t, J=8.4 Hz, 2H), 2.98 (t, J=8.0 Hz, 2H), 1.67 (s, 9H).

To a solution of Q-1 (480 mg, 0.9 mmol) in acetone (20 mL) was added DDQ (410 mg, 1.8 mmol) portionwise. The mixture was stirred at room temperature overnight, the solvent was evaporated and residue was purified by flash column chromatography to provide the product Q-2 (280 mg, 60%).

¹H NMR (400 MHz, CDCl₃) δ 8.49 (d, J=5.2 Hz, 1H), 8.33 (s, 1H), 7.65 (s, 1H), 7.56 (s, 1H), 7.50 (d, J=8.0 Hz, 1H), 7.31 (t, J=6.8 Hz, 2H), 7.26-7.25 (m, 1H), 7.14 (s, 1H), 7.09 (t, J=6.0 Hz, 1H), 6.91 (d, J=8.8 Hz, 1H), 6.71-6.68 (m, 2H), 6.57 (s, 1H), 5.41 (s, 2H), 3.64 (s, 3H), 1.67 (s, 9H).

Compound Q-2 (260 mg, 0.5 mmol) was added to 25% HCl in methanol solution. The mixture was stirred at 60° C. overnight, then concentrated to afford the product 1-[1-[(2-amino-4-pyridyl)-dideuteriomethyl]indol-4-yl]-3-(5-chloro-2-methoxyphenyl)urea hydrochloride (Q-3, 190 mg, 83%).

¹H NMR (400 MHz, DMSO-d₆) δ 12.08 (s, 1H), 9.23 (s, 1H), 8.79 (s, 1H), 8.28 (d, J=2.8 Hz, 1H), 8.19 (d, J=5.2 Hz, 1H), 7.70 (dd, J=6.0, 2.0 Hz, 1H), 7.54 (s, 1H), 7.50 (d, J=3.2 Hz, 1H), 7.05-6.96 (m, 4H), 6.79 (d, J=3.2 Hz, 1H), 6.73 (d, J=5.6 Hz, 1H), 6.47 (t, J=2.0 Hz, 1H), 5.80 (s, 2H), 3.91 (s, 3H).

A solution of Q-1 (100 mg, 0.19 mmol) in TFA (1 mL) was stirred at room temperature for 1 hour. TLC showed the reaction was complete. The solvent was removed under reduced pressure and to the residue was added ice water (5 mL). The pH of the mixture was adjusted to 7-8 using sat. NaHCO₃ (aq), DCM (1 mL) was then added. The mixture was stirred for 30 minutes. The product was collected by filtration to afford Q-4 (64 mg, 79%).

¹H NMR (400 MHz, DMSO-d₆) δ 8.68 (s, 1H), 8.65 (s, 1H), 8.23 (d, J=2.6 Hz, 1H), 7.83 (d, J=5.2 Hz, 1H), 7.19 (d, J=7.7 Hz, 1H), 7.02 (d, J=8.8 Hz, 1H), 6.99-6.86 (m, 2H), 6.50-6.38 (m, 2H), 6.22 (d, J=7.7 Hz, 1H), 5.84 (br, s, 2H), 3.88 (s, 3H), 3.32-3.26 (m, 2H), 2.89 (t, J=8.4 Hz, 2H).

### Examples 3 and 4

### 1-[1-[(2-Amino-4-pyridyl)-dideuteriomethyl]indolin-4-yl]-3-[5-chloro-2-(trideuteriomethoxy)phenyl]urea (Q-6 or Example 3) and 1-[1-[(2-amino-4-pyridyl)-dideuteriomethyl]indol-4-yl]-3-[5-chloro-2-(trideuteriomethoxy)phenyl]urea (Q-8 or Example 4)

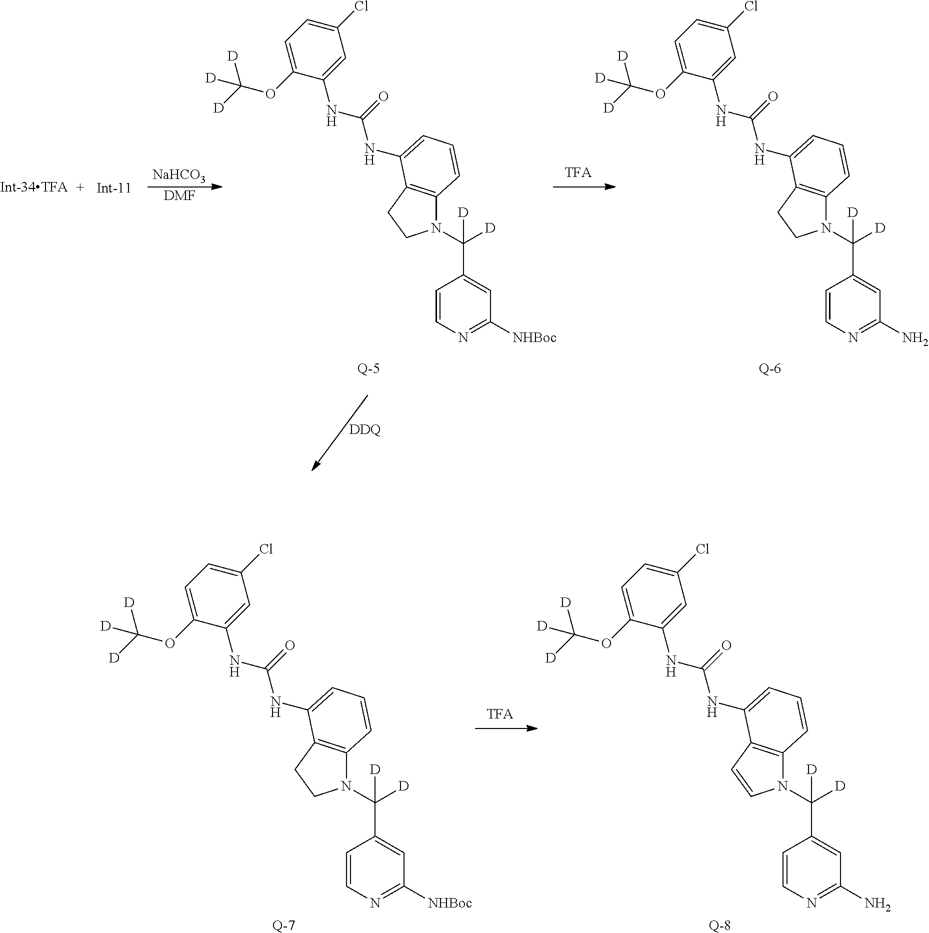

A mixture of Int-34.TFA (600 mg, 1.37 mmol) and NaHCO₃ (345 mg, 4.11 mmol) in DMF (6 mL) was stirred at room temperature for 10 minutes. Int-11 (475 mg, 1.65 mmol) was then added, and the mixture was stirred at room temperature for 3 hours. TLC showed the completion of the reaction. Ice water (20 mL) was added and the mixture was stirred for 10 minutes. The crude product was collected by filtration, which was purified by column chromatography (eluted with DCM/EtOAc: 20:1) to provide product Q-5 (700 mg, 96%).

A solution of Q-5 (100 mg, 0.19 mmol) in TFA (1 mL) was stirred at room temperature for 1 hour. TLC showed the reaction was complete. The solvent was removed under reduced pressure and to the residue was added ice water (5 mL). The pH of the mixture was adjusted to 7-8 using sat. NaHCO₃ (aq), DCM (1 mL) was then added. The mixture was stirred for 30 minutes. The product was collected by filtration to afford Q-6 (62 mg, 77%).

¹H NMR (400 MHz, DMSO-d₆) δ 8.68 (s, 1H), 8.65 (s, 1H), 8.21 (t, J=11.8 Hz, 1H), 7.83 (d, J=5.1 Hz, 1H), 7.20 (d, J=8.1 Hz, 1H), 7.02 (d, J=8.7 Hz, 1H), 6.94 (dt, J=16.0, 4.7 Hz, 2H), 6.49-6.38 (m, 2H), 6.22 (d, J=7.6 Hz, 1H), 5.85 (br, s, 2H), 3.31 (t, J=8.3 Hz, 2H), 2.89 (t, J=8.3 Hz, 2H).

A solution of Q-5 (300 mg, 0.5 mmol) and DDQ (260 mg, 1.1 mmol) in THF (20 mL) was stirred at room temperature for 10 minutes. TLC showed the reaction was complete. Ice water (40 mL) and 5N NaOH (aq, 0.5 mL) were added. The mixture was extracted with DCM (3×50 mL). The combined organic phases were washed with brine, dried over anhydrous sodium sulfate, filtered, concentrated under reduced pressure, and residue was trituated with MeOH (3 mL) to give the desired product Q-7 (200 mg, 67%).

A solution of Q-7 (200 mg, 0.38 mmol) in TFA (2 mL) was stirred at room temperature for 1 hour. TLC showed the reaction was complete. The solvent was removed under reduced pressure and to the residue was added ice water (5 mL). The pH of the mixture was adjusted to 7-8 using sat. NaHCO₃ (aq), DCM (1 mL) was then added. The mixture was stirred for 30 minutes. The product was collected by filtration to afford Q-8 (120 mg, 74%).

¹H NMR (400 MHz, DMSO-d₆) δ 9.18 (s, 1H), 8.79 (s, 1H), 8.29 (d, J=2.4 Hz, 1H), 7.80 (d, J=5.3 Hz, 1H), 7.77-7.64 (m, 1H), 7.41 (t, J=6.8 Hz, 1H), 7.11-6.93 (m, 4H), 6.72 (d, J=3.2 Hz, 1H), 6.28 (d, J=4.1 Hz, 1H), 6.05 (s, 1H), 5.88 (br, s, 2H).

### Examples 5 and 6

### 1-[1-[(2-Amino-4-pyridyl)methyl]indolin-4-yl]-3-[5-chloro-2-(trideuteriomethoxy)phenyl]urea (Q-9 or Example 5) and 1-[1-[(2-amino-4-pyridyl)methyl]indol-4-yl]-3-[5-chloro-2-(trideuteriomethoxy)phenyl]urea (Q-10 or Example 6)

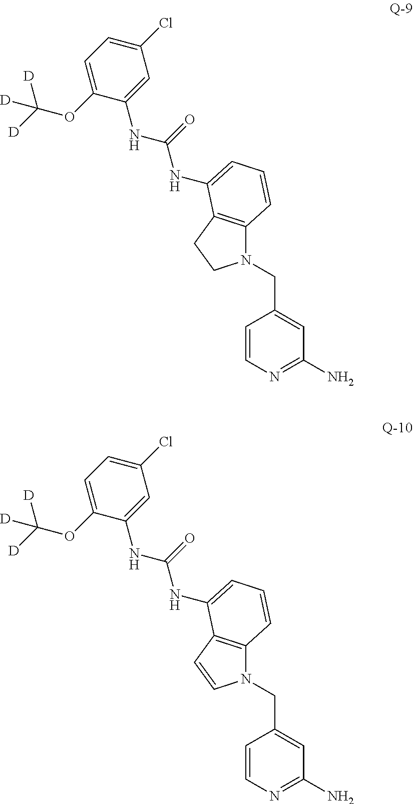 [SMILES: [2H]C([2H])([2H])Oc1ccc(Cl)cc1NC(=O)Nc1cccc2c1CCN2Cc1ccnc(N)c1 ; [2H]C([2H])([2H])Oc1ccc(Cl)cc1NC(=O)Nc1cccc2c1ccn2Cc1ccnc(N)c1]

Q-9 and Q-10 were prepared following the same procedures described for Examples 3 and 4 starting from Int-34.TFA and tert-butyl N-[4-(bromomethyl)-2-pyridyl]carbamate (synthesized according to M. O. Polla et al, Bioorg. Med. Chem. 2004, 12(5), 1151-1175). For Q-9: ¹H NMR (400 MHz, DMSO-d₆) δ 8.68 (s, 1H), 8.66 (s, 1H), 8.23 (d, J=2.2 Hz, 1H), 7.83 (d, J=5.1 Hz, 1H), 7.20 (d, J=8.2 Hz, 1H), 7.08-6.85 (m, 3H), 6.54-6.34 (m, 2H), 6.21 (d, J=7.8 Hz, 1H), 5.86 (br, s, 2H), 4.10 (s, 2H), 3.34 (t, J=8.3 Hz, 2H), 2.89 (t, J=8.3 Hz, 2H).

For Q-10: ¹H NMR (400 MHz, DMSO-d₆) δ 9.18 (s, 1H), 8.79 (s, 1H), 8.29 (d, J=2.5 Hz, 1H), 7.79 (d, J=5.2 Hz, 1H), 7.71 (dd, J=6.7, 1.6 Hz, 1H), 7.40 (d, J=3.2 Hz, 1H), 7.11-6.91 (m, 4H), 6.72 (d, J=3.1 Hz, 1H), 6.26 (d, J=4.3 Hz, 1H), 6.04 (s, 1H), 5.84 (br, s, 2H), 5.27 (s, 2H).

### Examples 7 and 8

### 1-[5-Chloro-2-(trideuteriomethoxy)phenyl]-3-[1-[dideuterio(1H-pyrrolo[2,3-b]pyridin-4-yl)methyl]indolin-4-yl]urea hydrochloride (Q-12 or Example 7) and 1-[5-chloro-2-(trideuteriomethoxy)phenyl]-3-[1-[dideuterio(1H-pyrrolo[2,3-b]pyridin-4-yl)methyl]indol-4-yl]urea hydrochloride (Q-14 or Example 8)

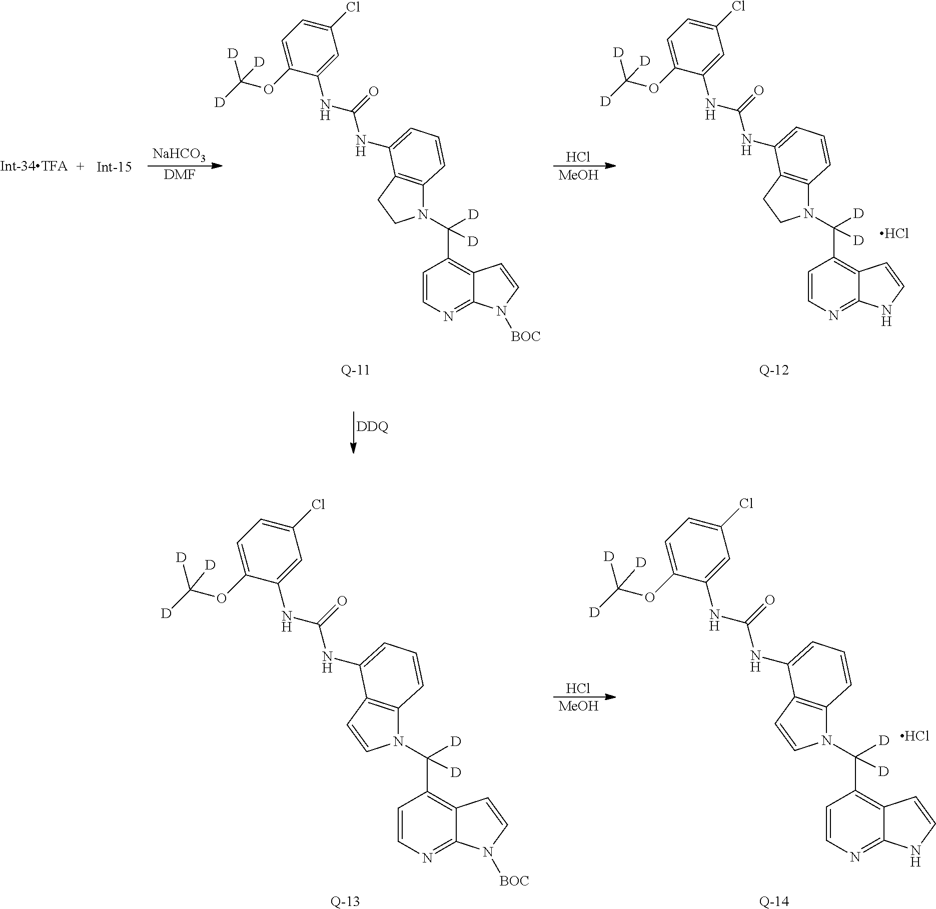

A mixture of Int-34.TFA (650 mg, 1.5 mmol) and NaHCO₃ (500 mg, 6 mmol) in DMF (10 mL) was stirred at room temperature for 10 minutes. Int-15 (400 mg, 1.5 mmol) was then added, and the mixture was stirred at room temperature for 2 days. TLC showed the reaction was complete. Water (100 mL) and EtOAc (100 mL) were added. Layers were separated and the aqueous layer was extracted with EtOAc (50 mL). The combined organic phases were washed with brine, dried over anhydrous sodium sulfate, filtered, concentrated under reduced pressure to give 480 mg of crude product, which was trituated with MeOH (5 mL) to afford the desired product Q-11 (350 mg, 42%) as a white solid.

¹H NMR (400 MHz, DMSO-d₆) δ 8.69 (s, 1H), 8.66 (s, 1H), 8.34 (d, J=4.9 Hz, 1H), 8.23 (d, J=2.5 Hz, 1H), 7.78 (s, 1H), 7.25 (dd, J=10.2, 6.6 Hz, 2H), 7.01 (d, J=8.7 Hz, 1H), 6.95 (ddd, J=11.4, 8.0, 4.7 Hz, 2H), 6.32 (d, J=7.4 Hz, 1H), 3.31 (t, J=8.5 Hz, 2H), 2.88 (t, J=8.3 Hz, 2H), 1.61 (s, 9H).

A solution of Q-11 (60 mg, 0.11 mmol) in 0.2 M HCl in MeOH (10 mL, 2 mmol) was stirred at room temperature for 2 days. TLC showed the reaction was complete. The solvent was removed under reduced pressure to give the crude product, which was trituated with EtOAc to afford the desired product Q-12 as a hydrochloride salt.

¹H NMR (400 MHz, DMSO-d₆) δ 12.60 (s, 1H), 8.78 (s, 1H), 8.74 (s, 1H), 8.37 (d, J=5.6 Hz, 1H), 8.23 (d, J=2.5 Hz, 1H), 7.68 (s, 1H), 7.37 (d, J=5.5 Hz, 1H), 7.25 (d, J=8.1 Hz, 1H), 6.95 (ddd, J=32.9, 25.4, 14.4 Hz, 4H), 6.29 (d, J=7.7 Hz, 1H), 3.40 (t, J=5.9 Hz, 2H), 2.94 (t, J=8.4 Hz, 2H).

MS (ESI⁺): m/z 475.2 [M+Na, ³⁵Cl]⁺, 477.2 [M+Na, ³⁷Cl]⁺.

A solution of Q-11 (110 mg, 0.2 mmol) and DDQ (90.8 mg, 0.4 mmol) in THF (10 mL) was stirred at room temperature. After reaction was complete (by TLC), DCM (50 mL), water (25 mL) and 5N NaOH (aq, 0.5 mL) were added. Layers were separated and the aqueous layer was extracted with DCM (50 mL). The combined organic phases were washed with brine, dried over anhydrous sodium sulfate, filtered, concentrated under reduced pressure, and residue was trituated with MeOH (1 mL) to give the desired product Q-13 (100 mg, 90%).

¹H NMR (400 MHz, DMSO-d₆) δ 9.20 (s, 1H), 8.79 (s, 1H), 8.28 (d, J=5.0 Hz, 2H), 7.78 (s, 1H), 7.74-7.65 (m, 1H), 7.50 (d, J=3.2 Hz, 1H), 7.09-6.93 (m, 4H), 6.82 (d, J=5.0 Hz, 1H), 6.76 (d, J=3.2 Hz, 1H), 1.59 (s, 9H).

A solution of Q-13 (100 mg, 0.18 mmol) in 0.2 M HCl in MeOH (7 mL, 1.4 mmol) was stirred at room temperature for 2 days. TLC showed the reaction was complete. The precipitated product was collected by filtration to afford the desired product Q-14 as a hydrochloride salt.

¹H NMR (400 MHz, DMSO-d₆) δ 12.43 (s, 1H), 9.31 (s, 1H), 8.86 (s, 1H), 8.26 (d, J=5.6 Hz, 1H), 7.72 (dd, J=5.2, 3.4 Hz, 1H), 7.62 (d, J=2.1 Hz, 1H), 7.53 (d, J=3.2 Hz, 1H), 7.08-6.93 (m, 4H), 6.82 (dd, J=12.6, 4.4 Hz, 2H), 6.54 (d, J=2.1 Hz, 1H).

MS (ESI⁺): m/z 473.1 [M+Na, ³⁵Cl]⁺, 475.2 [M+Na, ³⁷Cl]⁺.

### Examples 9 and 10

### 1-(5-Chloro-2-methoxyphenyl)-3-[1-[dideuterio(1H-pyrrolo[2,3-b]pyridin-4-yl)methyl]indolin-4-yl]urea hydrochloride (Q-15 or Example 9) and 1-(5-chloro-2-methoxyphenyl)-3-[1-[dideuterio(1H-pyrrolo[2,3-b]pyridin-4-yl)methyl]indol-4-yl]urea hydrochloride (Q-16 or Example 10)

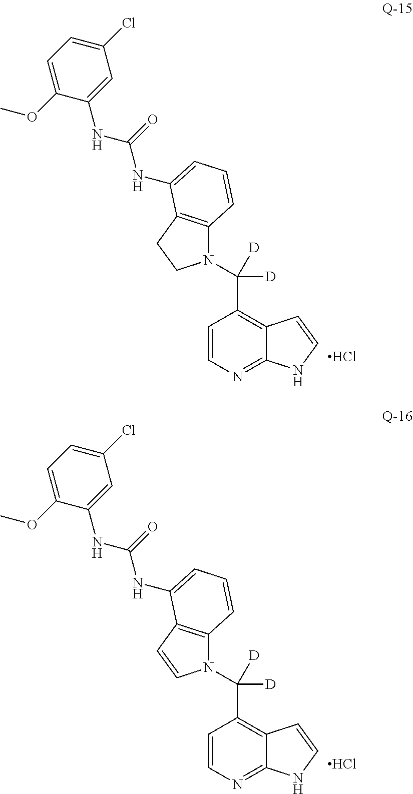 [SMILES: [2H]C([2H])(c1ccnc2[nH]ccc12)N1CCc2c(NC(=O)Nc3cc(Cl)ccc3OC)cccc21 ; Cl ; [2H]C([2H])(c1ccnc2[nH]ccc12)n1ccc2c(NC(=O)Nc3cc(Cl)ccc3OC)cccc21 ; Cl]

Q-15 and Q-16 were prepared following the same procedures described for Examples 7 and 8 starting from Int-30.TFA and Int-15.

For Q-15: ¹H NMR (400 MHz, DMSO-d₆) δ 11.48 (s, 1H), 8.66 (s, 1H), 8.60 (s, 1H), 8.21 (dd, J=9.3, 2.9 Hz, 2H), 7.98 (d, J=7.1 Hz, 1H), 7.43 (d, J=2.1 Hz, 1H), 7.20 (d, J=8.2 Hz, 1H), 7.07-6.92 (m, 4H), 6.50 (d, J=7.8 Hz, 1H), 3.87 (s, 3H), 3.24 (t, J=8.4 Hz, 2H), 2.78 (t, J=8.3 Hz, 2H).

MS (ESI⁺): m/z 472.1 [M+Na, ³⁵Cl]⁺, 474.2 [M+Na, ³⁷Cl]⁺.

For Q-16: ¹H NMR (400 MHz, DMSO-d₆) δ 11.58 (s, 1H), 9.12 (s, 1H), 8.76 (s, 1H), 8.27 (d, J=2.4 Hz, 1H), 8.17 (d, J=3.2 Hz, 1H), 7.85 (d, J=6.7 Hz, 1H), 7.68 (d, J=8.0 Hz, 1H), 7.62 (s, 1H), 7.48 (d, J=3.0 Hz, 1H), 7.33 (d, J=8.3 Hz, 1H), 7.09-6.93 (m, 4H), 6.64 (d, J=2.7 Hz, 1H), 5.48 (s, 1H), 3.90 (s, 3H).

MS (ESI⁺): m/z 470.1 [M+Na, ³⁵Cl]⁺, 472.1 [M+Na, ³⁷Cl]⁺.

### Examples 11 and 12

### 1-(5-Chloro-2-fluorophenyl)-3-[1-[dideuterio(1H-pyrrolo[2,3-b]pyridin-4-yl)methyl]indolin-4-yl]urea hydrochloride (Q-17 or Example 11) and 1-(5-chloro-2-fluorophenyl)-3-[1-[dideuterio(1H-pyrrolo[2,3-b]pyridin-4-yl)methyl]indol-4-yl]urea hydrochloride (Q-18 or Example 12)

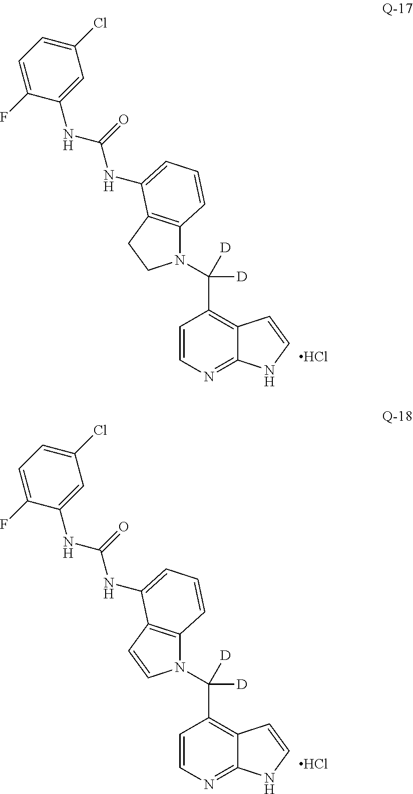 [SMILES: [2H]C([2H])(c1ccnc2[nH]ccc12)N1CCc2c(NC(=O)Nc3cc(Cl)ccc3F)cccc21 ; Cl ; [2H]C([2H])(c1ccnc2[nH]ccc12)n1ccc2c(NC(=O)Nc3cc(Cl)ccc3F)cccc21 ; Cl]

Q-17 and Q-18 were prepared following the same procedures described for Examples 7 and 8.

For Q-17: MS (ESI⁺): m/z 460.1 [M+Na, ³⁵Cl]⁺, 462.1 [M+Na, ³⁷Cl]⁺.

For Q-18: MS (ESI⁺): m/z 458.1 [M+Na, ³⁵Cl]⁺, 460.1 [M+Na, ³⁷Cl]⁺.

### Examples 13 and 14

### 1-[1-[Dideuterio(1H-pyrrolo[2,3-b]pyridin-4-yl)methyl]indolin-4-yl]-3-(m-tolyl)urea hydrochloride (Q-19 or Example 13) and 1-[1-[dideuterio(1H-pyrrolo[2,3-b]pyridin-4-yl)methyl]indol-4-yl]-3-(m-tolyl)urea hydrochloride (Q-20 or Example 14)

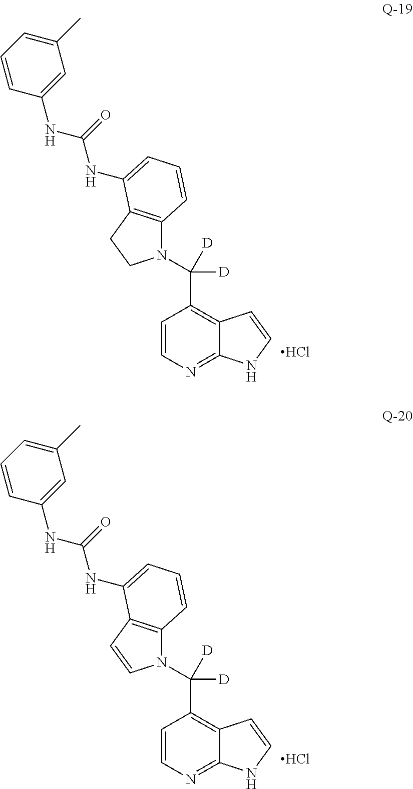 [SMILES: [2H]C([2H])(c1ccnc2[nH]ccc12)N1CCc2c(NC(=O)Nc3cccc(C)c3)cccc21 ; Cl ; [2H]C([2H])(c1ccnc2[nH]ccc12)n1ccc2c(NC(=O)Nc3cccc(C)c3)cccc21 ; Cl]

Q-19 and Q-20 were prepared following the same procedures described for Examples 7 and 8.

For Q-19: ¹H NMR (400 MHz, DMSO-d₆) δ 11.63 (s, 1H), 8.78 (s, 1H), 8.16 (d, J=4.9 Hz, 1H), 7.94 (s, 1H), 7.44 (d, J=2.4 Hz, 1H), 7.29 (s, 1H), 7.18 (dt, J=27.1, 7.8 Hz, 3H), 7.03 (d, J=4.8 Hz, 1H), 6.93 (t, J=8.0 Hz, 1H), 6.78 (d, J=7.4 Hz, 1H), 6.64 (s, 1H), 6.56 (dd, J=3.5, 1.8 Hz, 1H), 6.30 (d, J=7.2 Hz, 1H), 3.32 (t, J=8.3 Hz, 2H), 2.86 (t, J=8.3 Hz, 2H), 2.28 (s, 3H).

MS (ESI⁺): m/z 422.2 [M+Na]⁺.

For Q-20: ¹H NMR (400 MHz, DMSO-d₆) δ 12.29 (s, 1H), 9.32 (s, 1H), 8.87 (s, 1H), 8.24 (d, J=5.5 Hz, 1H), 7.72 (d, J=8.6 Hz, 1H), 7.59 (s, 1H), 7.51 (d, J=3.2 Hz, 1H), 7.34 (s, 1H), 7.29 (d, J=8.3 Hz, 1H), 7.16 (s, 1H), 7.00-6.97 (m, 2H), 6.86 (d, J=3.2 Hz, 1H), 6.78 (dd, J=5.4, 1.2 Hz, 2H), 6.50 (dd, J=3.5, 1.7 Hz, 1H), 2.28 (s, 3H).

MS (ESI⁺): m/z 420.2 [M+Na]⁺.

### Examples 15 and 16

### 1-[5-Chloro-2-(trideuteriomethoxy)phenyl]-3-[1-[deuterio(1H-pyrrolo[2,3-b]pyridin-3-yl)methyl]indolin-4-yl]urea (Q-23 or Example 15) and 1-[5-chloro-2-(trideuteriomethoxy)phenyl]-3-[1-[deuterio(1H-pyrrolo[2,3-b]pyridin-3-yl)methyl]indol-4-yl]urea (Q-24 or Example 16)

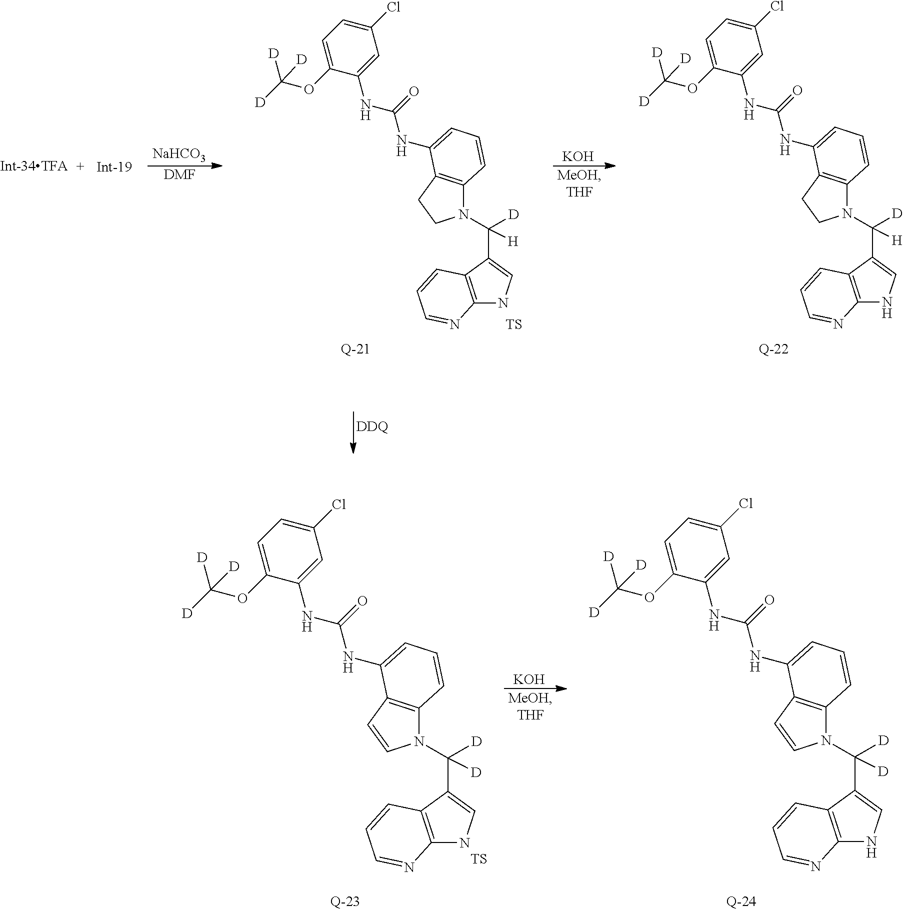

A mixture of Int-34.TFA (592.2 mg, 1.36 mmol) and NaHCO₃ (343 mg, 4.08 mmol) in DMF (10 mL) was stirred at room temperature for 10 minutes. Int-19 (500 mg, 1.36 mmol) was then added, and the mixture was stirred at room temperature for 1 hour. TLC showed the reaction was complete. Ice water (30 mL) was added. The precipitated product was collected by filtration to give 700 mg of the desired product Q-21 (yield: 85%) as a white solid, which was used directly in the next step without purification.

To a solution of Q-21 (100 mg, 0.164 mmol) in MeOH (5 mL) and THF (5 mL) was added KOH (40 mg, 0.714 mmol). The resulting mixture was stirred at room temperature for 3 hours. TLC showed the reaction was complete. The solvent was removed under reduced pressure, and to the residue was added ice water (10 mL). After stirred for 10 minutes, the mixture was filtered. The solid product was trituated with MeOH (3 mL) to afford the desired product Q-22 (60 mg, yield: 81%).

¹H NMR (400 MHz, DMSO-d₆) δ 11.49 (s, 1H), 8.67 (s, 1H), 8.60 (s, 1H), 8.21 (dd, J=9.2, 2.9 Hz, 2H), 7.98 (d, J=7.7 Hz, 1H), 7.43 (d, J=2.2 Hz, 1H), 7.20 (d, J=8.1 Hz, 1H), 7.07-6.92 (m, 4H), 6.50 (d, J=7.9 Hz, 1H), 4.34 (s, 1H), 3.24 (t, J=8.4 Hz, 2H), 2.78 (t, J=8.5 Hz, 2H).

MS (ESI⁺): m/z 474.1 [M+Na, ³⁵Cl]⁺, 476.1 [M+Na, ³⁷Cl]⁺.

A solution of Q-21 (340 mg, 0.56 mmol) and DDQ (255 mg, 1.12 mmol) in THF (20 mL) was stirred at room temperature for 10 minutes. TLC showed that the reaction was complete. Ice water (30 mL) and 5N NaOH (aq) were added to adjust the pH to 11-12. The mixture was extracted with EtOAc. The combined organic phases were washed with brine, dried over anhydrous sodium sulfate, filtered, concentrated under reduced pressure, and residue was trituated with MeOH (3 mL) to give the desired product Q-23 (240 mg, 71%).

To a solution of Q-23 (140 mg, 0.237 mmol) in MeOH (10 mL) and THF (30 mL) was added KOH (50 mg, 0.892 mmol). The resulting mixture was stirred at room temperature for 3 hours. TLC showed the reaction was complete. The solvent was removed under reduced pressure, and to the residue was added ice water (10 mL). After stirred for 10 minutes, the mixture was filtered. The solid product was trituated with MeOH (3 mL) to afford the desired product Q-24 (90 mg, yield: 87%).

¹H NMR (400 MHz, DMSO-d₆) δ 11.58 (s, 1H), 9.11 (s, 1H), 8.76 (s, 1H), 8.27 (d, J=2.5 Hz, 1H), 8.16 (d, J=3.3 Hz, 1H), 7.85 (d, J=7.9 Hz, 1H), 7.68 (d, J=7.7 Hz, 1H), 7.61 (d, J=2.3 Hz, 1H), 7.48 (d, J=3.2 Hz, 1H), 7.33 (d, J=8.3 Hz, 1H), 7.01 (ddt, J=20.9, 12.9, 8.2 Hz, 4H), 6.64 (d, J=2.8 Hz, 1H), 5.47 (s, 1H).

MS (ESI⁺): m/z 472.1 [M+Na, ³⁵Cl]⁺, 474.1 [M+Na, ³⁷Cl]⁺.

### Examples 17 and 18

### 1-(5-Chloro-2-methoxyphenyl)-3-[1-[deuterio(1H-pyrrolo[2,3-b]pyridin-3-yl)methyl]indolin-4-yl]urea (Q-25 or Example 17) and 1-(5-chloro-2-methoxyphenyl)-3-[1-[deuterio(1H-pyrrolo[2,3-b]pyridin-3-yl)methyl]indol-4-yl]urea (Q-26 or Example 18)

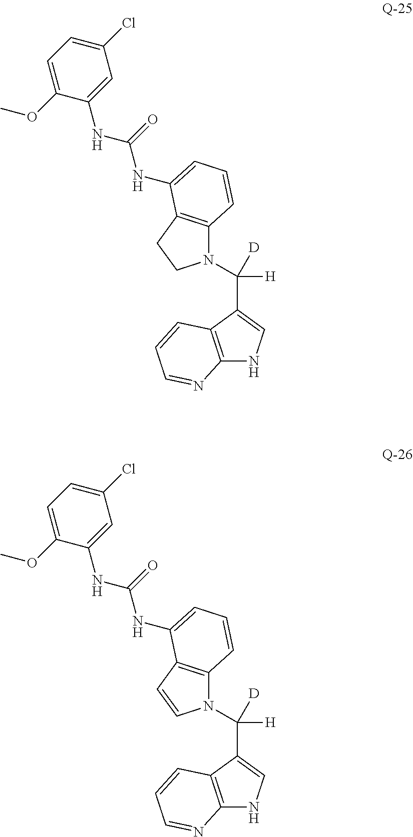 [SMILES: [2H]C(c1c[nH]c2ncccc12)N1CCc2c(NC(=O)Nc3cc(Cl)ccc3OC)cccc21 ; [2H]C(c1c[nH]c2ncccc12)n1ccc2c(NC(=O)Nc3cc(Cl)ccc3OC)cccc21]

Q-25 and Q-26 were prepared following the same procedures described for Examples 15 and 16.

For Q-25: ¹H NMR (400 MHz, DMSO-d₆) δ 11.48 (s, 1H), 8.66 (s, 1H), 8.60 (s, 1H), 8.21 (dd, J=9.3, 2.9 Hz, 2H), 7.98 (d, J=7.1 Hz, 1H), 7.43 (d, J=2.1 Hz, 1H), 7.20 (d, J=8.2 Hz, 1H), 7.07-6.92 (m, 4H), 6.50 (d, J=7.8 Hz, 1H), 4.34 (s, 1H), 3.87 (s, 3H), 3.24 (t, J=8.4 Hz, 2H), 2.78 (t, J=8.3 Hz, 2H).

MS (ESI⁺): m/z 471.1 [M+Na, ³⁵Cl]⁺, 473.1 [M+Na, ³⁷Cl]⁺.

For Q-26: ¹H NMR (400 MHz, DMSO-d₆) δ 11.58 (s, 1H), 9.12 (s, 1H), 8.76 (s, 1H), 8.27 (d, J=2.4 Hz, 1H), 8.17 (d, J=3.2 Hz, 1H), 7.85 (d, J=6.7 Hz, 1H), 7.68 (d, J=8.0 Hz, 1H), 7.62 (s, 1H), 7.48 (d, J=3.0 Hz, 1H), 7.33 (d, J=8.3 Hz, 1H), 7.09-6.93 (m, 4H), 6.64 (d, J=2.7 Hz, 1H), 5.48 (s, 1H), 3.90 (s, 3H).

MS (ESI⁺): m/z 469.1 [M+Na, ³⁵Cl]⁺, 471.1 [M+Na, ³⁷Cl]⁺.

### Examples 19 and 20

### 1-(5-Chloro-2-fluorophenyl)-3-[1-[deuterio(1H-pyrrolo[2,3-b]pyridin-3-yl)methyl]indolin-4-yl]urea (Q-27 or Example 19) and 1-(5-chloro-2-fluorophenyl)-3-[1-[deuterio(1H-pyrrolo[2,3-b]pyridin-3-yl)methyl]indol-4-yl]urea (Q-28 or Example 20)

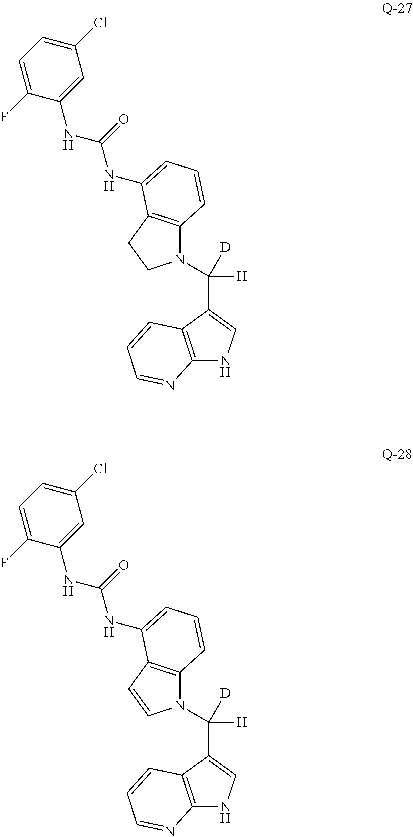 [SMILES: [2H]C(c1c[nH]c2ncccc12)N1CCc2c(NC(=O)Nc3cc(Cl)ccc3F)cccc21 ; [2H]C(c1c[nH]c2ncccc12)n1ccc2c(NC(=O)Nc3cc(Cl)ccc3F)cccc21]

Q-27 and Q-28 were prepared following the same procedures described for Examples 15 and 16.

For Q-27: ¹H NMR (400 MHz, DMSO-d₆) δ 11.49 (s, 1H), 9.01 (d, J=2.5 Hz, 1H), 8.43 (s, 1H), 8.29 (dd, J=7.1, 2.6 Hz, 1H), 8.20 (dd, J=4.6, 1.5 Hz, 1H), 7.98 (d, J=6.9 Hz, 1H), 7.43 (d, J=2.2 Hz, 1H), 7.33-7.18 (m, 2H), 7.07-6.94 (m, 3H), 6.51 (d, J=7.5 Hz, 1H), 4.35 (s, 1H), 3.26 (t, J=8.4 Hz, 2H), 2.78 (t, J=8.4 Hz, 2H).

MS (ESI⁺): m/z 459.1 [M+Na, ³⁵Cl]⁺, 461.1 [M+Na, ³⁷Cl]⁺.

For Q-28: ¹H NMR (400 MHz, DMSO-d₆) δ 11.58 (s, 1H), 9.06 (s, 1H), 8.95 (s, 1H), 8.34 (dd, J=7.0, 2.5 Hz, 1H), 8.16 (d, J=3.5 Hz, 1H), 7.85 (d, J=7.0 Hz, 1H), 7.67 (d, J=7.6 Hz, 1H), 7.62 (s, 1H), 7.51 (d, J=3.1 Hz, 1H), 7.39-7.25 (m, 2H), 7.02 (ddd, J=12.6, 11.8, 6.3 Hz, 3H), 6.56 (d, J=2.9 Hz, 1H), 5.48 (s, 1H).

MS (ESI⁺): m/z 457.1 [M+Na, ³⁵Cl]⁺, 459.1 [M+Na, ³⁷Cl]⁺.

### Examples 21 and 22

### 1-[1-[Deuterio(1H-pyrrolo[2,3-b]pyridin-3-yl)methyl]indolin-4-yl]-3-(m-tolyl)urea (Q-29 or Example 21) and 1-[1-[deuterio(1H-pyrrolo[2,3-b]pyridin-3-yl)methyl]indol-4-yl]-3-(m-tolyl)urea (Q-30 or Example 22)

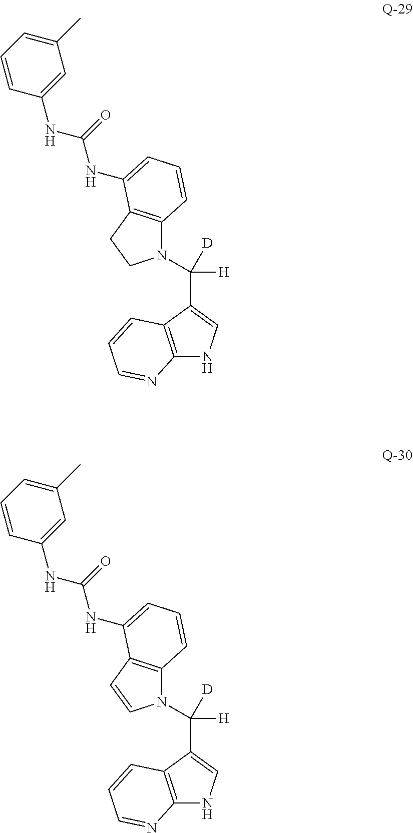 [SMILES: [2H]C(c1c[nH]c2ncccc12)N1CCc2c(NC(=O)Nc3cccc(C)c3)cccc21 ; [2H]C(c1c[nH]c2ncccc12)n1ccc2c(NC(=O)Nc3cccc(C)c3)cccc21]

Q-29 and Q-30 were prepared following the same procedures described for Examples 15 and 16.

For Q-29: ¹H NMR (400 MHz, DMSO-d₆) δ 11.48 (s, 1H), 8.76 (s, 1H), 8.20 (d, J=3.4 Hz, 1H), 7.98 (d, J=7.7 Hz, 1H), 7.89 (s, 1H), 7.43 (s, 1H), 7.27 (s, 1H), 7.16 (dt, J=25.5, 7.9 Hz, 3H), 7.04 (dd, J=7.8, 4.7 Hz, 1H), 6.96 (t, J=8.0 Hz, 1H), 6.77 (d, J=7.1 Hz, 1H), 6.48 (d, J=7.7 Hz, 1H), 4.34 (s, 1H), 3.25 (t, J=8.3 Hz, 2H), 2.76 (t, J=8.2 Hz, 2H), 2.26 (s, 3H).

MS (ESI⁺): m/z 421.2 [M+Na]⁺.

For Q-30: ¹H NMR (400 MHz, DMSO-d₆) δ 11.58 (s, 1H), 8.80 (s, 1H), 8.43 (s, 1H), 8.16 (d, J=3.5 Hz, 1H), 7.85 (d, J=7.9 Hz, 1H), 7.69-7.57 (m, 2H), 7.49 (d, J=2.9 Hz, 1H), 7.31 (d, J=8.5 Hz, 2H), 7.24 (d, J=8.4 Hz, 1H), 7.16 (t, J=7.6 Hz, 1H), 7.08-6.94 (m, 2H), 6.78 (d, J=7.0 Hz, 1H), 6.54 (d, J=3.0 Hz, 1H), 5.47 (s, 1H), 2.28 (s, 3H).

MS (ESI⁺): m/z 419.2 [M+Na]⁺.

### Example 23

### VEGFR2 Cell-Based Assays

A standard cell-based assay for VEGFR2 kinase activity can be used to test or screen the compounds of this invention and to identify VEGFR2 antagonists. In this example, ELISA kits (R&D Systems, Inc., Minneapolis, Minn. 55413, USA) were used.

The activity of VEGFR2 in cells depended on its autophosphorylation level by various factors including growth factors. In order to induce the cell to produce VEGFR2, full length of VEGFR2 sequence was cloned into PC-DNA3.1 vector, the plasmid was then transfected into CHO cells. After 48 hours, the expression of VEGFR2 in transfected CHO cells was confirmed by regular western blotting.

A compound to be tested was first dissolved in DMSO to a concentration of 10 mM and stored at −20° C. One day before the test, CHO cells transfected with full length VEGFR2 plasmid were grown in Dulbecco's Modified Eagle Medium (DMEM) without the presence of serum. On the day of the test, the compounds with various concentrations in DMSO (from 0.5 nM to 500 nM) were added into DMEM. Two hours later, 20% of FBS (Fetal Bovine Serum) was added into the medium to stimulate the production of phosphor-VEGFR2. Cell lysate from approximately 5×10⁵ transfected CHO cells were reacted with anti-phosphor VEGFR2 or anti-total VEGFR2 antibody in 96-well plates, in the presence of detection antibody. The phosphor-VEGFR2 or total VEGFR2 were detected with Streptavidin-HRP(R&D Systems), followed by chemiluminescent detection. The level of VEGFR2 kinase activity was quantified as the ratio between the amount of phosphor- and that of total VEGFR2 proteins.

The percentage of VEGFR2 kinase activity that remained in the presence of a compound at the concentration of 100 nM is listed in the following table for each tested compound, wherein A indicates that the percentage of kinase activity remains is below 70% and B indicates that the percentage is greater or equal to 70%.

| | % VEGFR2 kinase |
|---|---|
| Example | activity remains |
| 1 | A |
| 2 | A |
| 3 | A |
| 4 | A |
| 5 | A |
| 6 | B |
| 7 | A |
| 8 | A |
| 9 | A |
| 10 | A |
| 11 | A |
| 12 | A |
| 13 | A |
| 14 | A |
| 15 | A |
| 16 | A |
| 17 | A |
| 18 | A |
| 19 | A |
| 20 | A |
| 21 | A |
| 22 | A |

### Example 24

### PDGFRβ Cell-Based Assays

A standard cell-based assay for PDGFRβ kinase activity can be used to test or screen the compounds of this invention and to identify PDGFRβ antagonists. In this example, ELISA kits (R&D Systems, Inc., Minneapolis, Minn. 55413, USA) were used.

The activity of PDGFRβ in cells depended on its autophosphorylation level by various factors including growth factors. In order to induce the cell to produce PDGFRβ, full length of PDGFRβ sequence was cloned into PC-DNA3.1 vector, the plasmid was then transfected into CHO cells. After 48 hours, the expression of PDGFRβ in transfected CHO cells was confirmed by regular western blotting.

A compound to be tested was first dissolved in DMSO to a concentration of 10 mM and stored at −20° C. One day before the screening, CHO cells transfected with full length PDGFRβ plasmid were grown in DMEM medium without the presence of serum. On the day of screening procedure, the compounds with various concentration in DMSO (from 0.5 nM to 500 nM) were added into DMEM medium, two hours later, 20% of FBS (Fetal Bovine Serum) was added into the medium to stimulate the production of phosphor-PDGFRβ. Cell lysate from approximately 5×10⁵ transfected CHO cells were reacted with anti-phosphor PDGFRβ or anti-total PDGFRβ antibody in 96 well plates, in the presence of detection antibody. The phosphor-PDGFRβ or total PDGFRβ were detected with Streptavidin-HRP (R&D Systems), followed by chemiluminescent detection. The level of PDGFRβ kinase activity was quantified as the ratio between the amount of phosphor- and that of total PDGFRβ proteins.

The percentage of PDGFRβ kinase activity that remained in the presence of a compound at the concentration of 100 nM is listed in the following table for each tested compound, wherein A indicates that the percentage of kinase activity remains is below 70% and B indicates that the percentage is greater or equal to 70%.

| | % PDGFRβ kinase |
|---|---|
| Example | activity remains |
| 1 | A |
| 2 | A |
| 3 | B |
| 4 | B |
| 5 | A |
| 6 | A |
| 7 | A |
| 8 | A |
| 9 | A |
| 10 | B |
| 11 | A |
| 12 | B |
| 13 | A |
| 14 | A |
| 15 | B |
| 16 | B |
| 17 | A |
| 18 | A |
| 19 | B |
| 20 | A |
| 21 | A |
| 22 | A |

### Example 25

### C-Met Cell-Based Assays

A standard cell-based assay for c-Met kinase activity can be used to test or screen the compounds of this invention and to identify c-Met antagonists. In this example, ELISA kits (R&D Systems, Inc., Minneapolis, Minn. 55413, USA) were used.

The activity of c-Met in cells depended on its autophosphorylation level by it specific growth factor-Hepatocyte growth factor (HGF). In order to induce the cell to produce c-Met, full length of c-Met sequence was cloned into PC-DNA3.1 vector, the plasmid was then transfected into CHO cells. After 48 hours, the expression of c-Met in transfected CHO cells was confirmed by regular western blotting.

A compound to be tested was first dissolved in DMSO to a concentration of 10 mM and stored at −20° C. One day before the screening, CHO cells transfected with full length c-Met plasmid were grown in DMEM medium without the presence of serum. On the day of screening procedure, the compounds with various concentration in DMSO (from 0.5 nM to 500 nM) were added into DMEM medium, two hours later, Hepatocyte growth factor (HGF) was added into the medium at the concentration of 100 ng/mL to stimulate the production of phosphor-c-Met. Cell lysate from approximately 5×10⁵ transfected CHO cells were reacted with anti-phosphor c-Met or anti-total c-Met antibody in 96 well plates, in the presence of detection antibody. The phosphor-c-Met or total c-Met were detected with Streptavidin-HRP (R&D Systems), followed by chemiluminescent detection. The level of c-Met kinase activity was quantified as the ratio between the amount of phosphor- and that of total c-Met proteins.

The percentage of c-Met kinase activity that remained in the presence of a compound at the concentration of 100 nM is listed in the following table for each tested compound, wherein A indicates that the percentage of kinase activity remains is below 70% and B indicates that the percentage is greater or equal to 70%.

| | % C-Met kinase |
|---|---|
| Example | activity remains |
| 1 | B |
| 2 | B |
| 3 | A |
| 4 | A |
| 5 | A |
| 6 | A |
| 7 | A |
| 8 | A |
| 9 | A |
| 10 | A |
| 11 | A |
| 12 | A |
| 13 | A |
| 14 | A |
| 15 | A |
| 16 | A |
| 17 | A |
| 18 | A |
| 19 | B |
| 20 | A |
| 21 | B |
| 22 | A |

### Example 26

### EphB4 Cell-Based Assays

A standard image-based quantitative Western blotting assay for EphB4 kinase activity can be used to test or screen the compounds of this invention and to identify EphB4 antagonists.

The kinase activity of EphB4 in cells depends on its autophosphorylation level stimulated by the binding to its ligand Ephrin-B2. High level of EphB4 autophosphorylation in PC-3 human prostate cancer cells (ATCC, USA) induced by the application of Ephrin-B2 to the cell culture can be observed by regular Western blotting assay.

A compound to be tested was first dissolved in DMSO to a concentration of 10 mM and stored at −20° C. On the day of screening procedure, the compound in DMSO was added into DMEM medium to a final concentration of 100 nM. Two hours later, Ephrin-B2 (4 mg/ml) was added into DMEM medium to stimulate the production of phosphor-EphB4. The phosphor-EphB4 was detected based on its molecular weight and with anti-phosphor protein antibody PY-20 in cell lysate from approximately 5×10⁵ PC-3 cells by Western blotting followed by imaging quantification. The relative level of EphB4 kinase activity was quantified as the ratio between the amount of phosphor-EphB4 and that of total Actin proteins.

The percentage of EphB4 kinase activity that remained in the presence of a compound at the concentration of 100 nM is listed in the following table for each tested compound, wherein A indicates that the percentage of kinase activity remains is below 70% and B indicates that the percentage is greater or equal to 70%.

| | % EphB4 kinase |
|---|---|
| Example | activity remains |
| 1 | A |
| 2 | A |
| 3 | A |
| 4 | A |
| 5 | A |
| 6 | A |
| 7 | B |
| 8 | B |
| 9 | B |
| 10 | B |
| 11 | A |
| 12 | A |
| 13 | B |
| 14 | A |
| 15 | B |
| 16 | B |
| 17 | A |
| 18 | A |
| 19 | B |
| 20 | B |
| 21 | B |
| 22 | B |

### Example 27

### Liver Microsomal Stability Assays

A standard liver microsomal stability assay can be used to test or screen the compounds of this invention and to identify compounds with enhanced metabolic stability due to the enrichment of deuterium in the compounds. Liver microsome proteins prepared from different species such as mouse and rat were obtained from a commercial source (Research Institute for Liver Diseases, Shanghai, China). The assay was conducted in 1.5% potassium hydrogenphosphate (K₂HPO4) in the presence of liver microsome proteins at 1 mg/ml and supplemented with an NADH generating system (3.3 mM NADPH, 3.3 mM glucose-6-phosphate, 6 units/ml G6PDase, and 3.3 mM magnesium chloride). Test compound was added into the assay mixture to a final concentration of 40 μM and incubated at 37° C. for 0 or 30 minutes. The reaction was stopped by the addition of 20 μM Bifonazole/CAN. Samples were then centrifuged at 14000 rpm for 10 minutes to precipitate proteins. Supernatant were then transferred to microcentrifuge tubes and subject to reverse phase HPLC analysis to determine the concentration of test compound.

The percentage of compounds that remained in the assay reactions after a thirty-minute incubation at 37° C. is calculated as the ratio between the amount of compound remained after a thirty-minute and that after a zero-minute incubation. The results are listed in the following table for each compound tested, wherein A indicates that the percentage of a compound remains is greater than that of its deuterium non-enriched counterpart and B indicates a result otherwise.

% of compound remained at
the 30^{th} minutes

| | Mouse liver | Rat liver |
|---|---|---|
| Example | microsome | microsome |
| 1 | A | A |
| 2 | A | A |
| 3 | B | A |
| 4 | B | A |
| 5 | B | A |
| 6 | B | A |
| 7 | A | A |
| 8 | A | A |
| 9 | A | A |
| 10 | A | B |
| 11 | B | B |
| 12 | B | A |
| 13 | A | A |
| 14 | A | A |
| 15 | B | A |
| 16 | A | A |
| 17 | A | A |
| 18 | A | A |
| 19 | B | B |
| 20 | B | A |
| 21 | A | A |
| 22 | A | B |

### Example 28

### Oral Bioavailability Study—Group 1

Oral pharmacokinetic studies were performed on Example 2. Specifically, this compound was dissolved in PEG-400 (2:1 mixture of PEG400 and 0.01N HCL (v/v)) and administered orally to male ICR mice at a dose of 50 mg/kg. Mice blood was obtained via eyeball enucleation at 0.25, 0.5, 1, 2, 3, 4, 6, 8, 12 and 24 hours after administration. The concentration of Example 2 in blood was determined using appropriate internal standards and reverse phase HPLC with UV detection.

Control blood samples were also used to identify compound-specific peaks. The oral bioavailability was determined by comparing the dose corrected areas under plasma-concentration time curve (AUC) to infinity for Example 2, given orally and intravenously in mice.

The studies demonstrated that substantial amounts of Example 2 were detected in the blood of the mice 0.25 hour after oral dosing. The compound showed excellent pharmacokinetic properties with the oral bioavailability more than 88% in these mice. Furthermore, its long half-life (2.3-3.5 hours) suggests prolonged activity which in turn will diminish the need for frequent dosing regimens. All these pharmacokinetic properties demonstrated that Example 2 has excellent drug properties, especially good oral bioavailability.

### Example 29

### Oral Bioavailability Study—Group 2

Oral pharmacokinetic studies on rats were performed on Example 2, this compound was dissolved in PEG-400 (2:1 mixture of PEG400 and 0.01N HCL (v/v)) and administered orally to male SD rat at a dose of 50 mg/kg. Rat blood was obtained from tail vein at 0.25, 0.5, 1, 2, 3, 4, 6, 8, 12, and 24 hours after administration. The blood concentration of Example 2 was determined using appropriate internal standards and reverse phase HPLC with UV detection.

Control blood samples were also run to identify compound-specific peaks. The oral bioavailability was determined by comparing the dose corrected areas under plasma-concentration time curve (AUC) to infinity for Example 2, given orally and intravenously in rats.

These studies demonstrate that substantial amounts of Example 2 were detected in the blood of the mice 0.25 hours after oral dosing and that it also had excellent pharmacokinetic properties with the oral bioavailability more than 67% on these rats. Furthermore, the long half-life (2-3 hours) suggests prolonged activity which will diminish the need for frequent dosing regimens. These pharmacokinetic properties demonstrated that Example 2 had excellent drug properties, especially good oral availability with low toxicity.

### Example 30

### In Vivo Testing of Anti-Tumor Efficacy

Female (nu/nu) Balb/c athymic mice at 4-6 weeks of age were injected subcutaneously (s.c.) with MDA-MB-231 human breast cancer cells, A549 human non-small cell lung cancer cells, BEL7401 human liver cancer cells, HT-29 human colon cancer cells, or MKN-45 human gastric cancer cells (ATCC, 5×10⁶ cells suspended in 100 μl DMEM medium). Treatment was initiated after the tumor mass grown from subcutaneous implanted tumor cells reaches a median volume of 200-400 mm³. Mice were randomized into groups with three in each group such that the median tumor volume is nearly equal among all groups. Each group was treated either with compounds (dissolved in 10% ethanol, 20% cremorphor, and 70% saline) or without compounds (vehicle only, 10% ethanol, 20% cremorphor, and 70% saline) once per day at dose 50 mg/kg by oral gavage. Tumor volumes were assessed at least twice weekly by caliper measurement from the start of treatment. Tumor volume was calculated using the formula ½×L×W² (L: length of tumor's long axis, W: length of tumor's short axis). Treatment was applied for at least two weeks or until the tumor volume reaches a size of ˜2500 mm³. Mice were humanly sacrificed after the experiment.

The efficacy of compounds in reducing tumor growth was assessed by the index Tumor Growth Inhibition (TGI). TGI for a given treatment period was calculated using the group mean tumor volumes according to the formula:

TGI=100×(V_{vehicle}_{_{—}}_{group}−V_{Treatment}_{_{—}}_{group})÷V_{Vehicle}_{_{—}}_{group}

wherein V_{Vehicle}_{_{—}}_{group} representing the mean tumor volume for the group treated with vehicle and V_{Treatment}_{_{—}}_{group} representing the mean tumor volume of the group treated with a compound.

The Tumor Growth Inhibition (TGI) for the compounds of this invention in in vivo anti-tumor efficacy test is listed in the following table, wherein B indicates that the TGI for the compound of this invention is between 0% and 50% and A indicates that the TGI is greater or equal to 50%.

The following table summarizes the results of efficacy studies of Example 2:

| Indication (group × | | Dosage | Tumor Growth |
|---|---|---|---|
| sample size) | Cancer cell line | (mg/kg) | Inhibition (TGI) |
| Breast cancer (n = 2 × 3) | MDA-MB-231 | 50 | A |
| Gastric cancer (n = 2 × 3 ) | MKN-45 | 50 | A |
| Liver cancer (n = 2 × 3) | BEL7404 | 50 | A |
| Non-small cell lung | A549 | 50 | A |
| cancer (n = 2 × 3) | | | |
| Colon cancer (n = 2 × 3) | HT-29 | 50 | A |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. All publications referenced herein are incorporated by reference in their entireties.

1. A compound of Formula I or a pharmaceutically acceptable salt thereof, 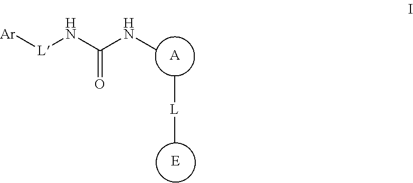
wherein:
E is E1, E2 or E3: 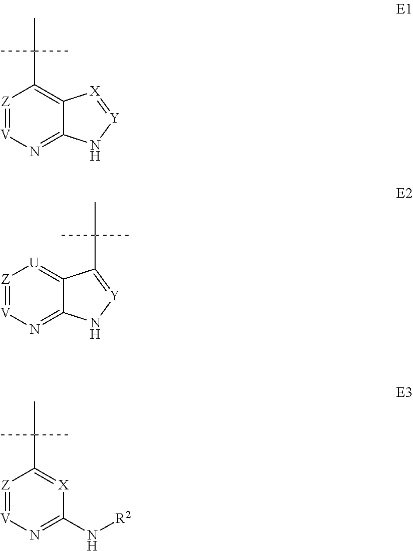
U, V, X, Y, and Z are each independently N or C—R¹;
L is a C₁₋₈alkylene, optionally deuterium-enriched and optionally substituted with one or more independent R³;
R² is H, C₁₋₈alkyl, C₆₋₁₂aryl, C₅₋₁₂heteroaryl, —C(═O)—C₁₋₈alkyl, —C(═O)—C₆₋₁₂aryl, or —C(═O)—C₅₋₁₂heteroaryl, each of which is optionally substituted with one or more independent Q¹;
L′ is a covalent bond, —C(═O)—, —C(═O)—C₁₋₈alkylene, or C₁₋₈alkylene, each of which is optionally substituted with one or more independent R⁴;
A is A1, A2, or A3: 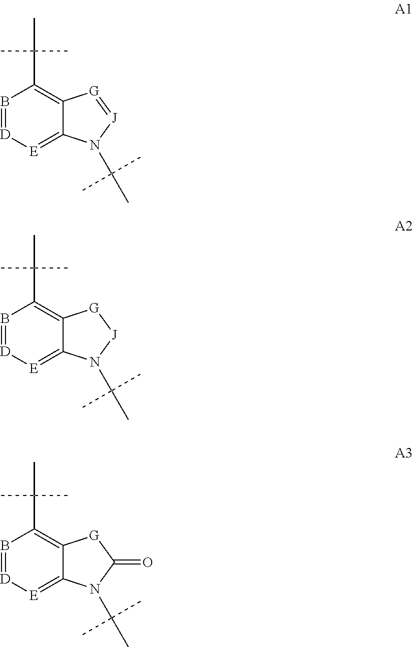
wherein B, D, E, G, and J are each independently N or CH;
each of A1, A2, and A3 is optionally substituted with one or more independent R⁵;
Ar is aryl or heteroaryl, each of which is optionally substituted with one or more independent R⁶;
R¹, R³, R⁴, R⁵, and R⁶ are each independently H, halo, —CN, —CF₃, —NO₂, —NH₂, —OH, —OCF₃, C₁₋₈alkyl-O—, —CO₂H, C₁₋₈alkyl, C₂₋₈alkynyl, C₃₋₁₂cycloalkyl, C₃₋₁₂heterocycloalkyl, C₆₋₁₂arylC₁₋₈alkyl, or C₅₋₁₂heteroarylC₁₋₈alkyl, each of which is optionally substituted with one or more independent Q²;
or R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q²;
Q¹ and Q² are each independently H, halo, —CN, —CF₃, —OCF₃, —NO₂, oxo, C₁₋₈alkyl, C₂₋₈alkenyl, C₂₋₈alkynyl, C₃₋₁₂cycloalkyl, C₃₋₁₂heterocycloalkyl, C₆₋₁₂aryl, C₅₋₁₂heteroaryl, C₈₋₁₂heterocycloaryl, —OR⁷, —S(O)ₙR⁸, —NR⁹R¹⁰, —SO₂NR⁹R¹⁰, —C(O)R¹¹, —C(O)NR⁹R¹⁰, —C(O)OR⁷, —OC(O)R¹¹, —NR⁹C(O)R¹¹, NR⁹S(O)₂R¹², —NR¹³C(O)NR⁹R¹⁰, —NR¹³S(O)₂NR⁹R¹⁰, or —NR¹³S(O)NR⁹R¹⁰, each of which is optionally substituted with one or more independent H, halo, —CN, —OH, —NH₂, —NO₂, oxo, —CF₃, —OCF₃, —CO₂H, —S(O)ₙH, C₁₋₈alkyl, C₆₋₁₂aryl, C₅₋₁₂heteroaryl, C₃₋₁₂cycloalkyl, C₃₋₁₂heterocycloalkyl, C₈₋₁₂heterocycloaryl, or —O—C₁₋₈alkyl, each of which is optionally partially or fully halogenated;
R⁷, R⁸, R⁹, R¹⁰, R¹¹, R¹², and R¹³ are each independently H, C₁₋₈alkyl, C₂₋₈alkenyl, C₂₋₈alkynyl, C₃₋₁₂cycloalkyl, C₃₋₁₂heterocycloalkyl, C₆₋₁₂aryl, C₅₋₁₂heteroaryl, or C₈₋₁₂heterocycloaryl;
or when in —NR⁹R¹⁰, R⁹ and R¹⁰, together with the nitrogen atom to which they are attached, form a 3- to 12-membered saturated or unsaturated ring, wherein the ring optionally includes one or more heteroatoms each independently being O, N, or S(O)ₙ;
n is 0, 1, or 2;
when L is not deuterium-enriched, then Ar must be substituted with one or more independent R⁶, and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

2. The compound of claim 1, wherein X, Y, Z, V, and U are each independently C—R¹:

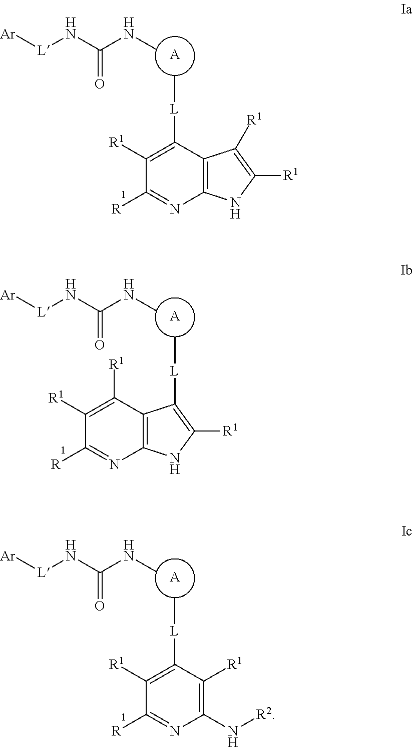

3. The compound of claim 2, wherein L′ is a covalent bond.

4. The compound of claim 2, wherein Ar is C₆₋₁₂aryl or C₅₋₁₂heteroaryl, each of which is optionally substituted with one or more independent R⁶.

5. The compound of claim 4, wherein Ar is substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

6. The compound of claim 2, wherein L has 1 to 4 carbon atoms and is optionally deuterium-enriched and optionally substituted with one or more independent R³.

7. The compound of claim 6, wherein L has 1 or 2 carbon atoms and is optionally deuterium-enriched and optionally substituted with one or more independent R³.

8. The compound of claim 7, wherein L has one carbon atom and is optionally deuterium-enriched and optionally substituted with one or more independent R³.

9. The compound of claim 7, wherein L is —CD₂-, —CHD-, or —CD₂CD₂-.

10. The compound of claim 7, wherein L is —CH₂—; Ar is C₆₋₁₂aryl or C₅₋₁₂heteroaryl and substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

11. The compound of claim 2, wherein A is A1-a, A1-b, A1-c, A1-d, A1-e, A1-f, A1-g, A1-h, A1-i, A2-a, A2-b, A2-c, A2-d, A2-e, A2-f, or A2-g: 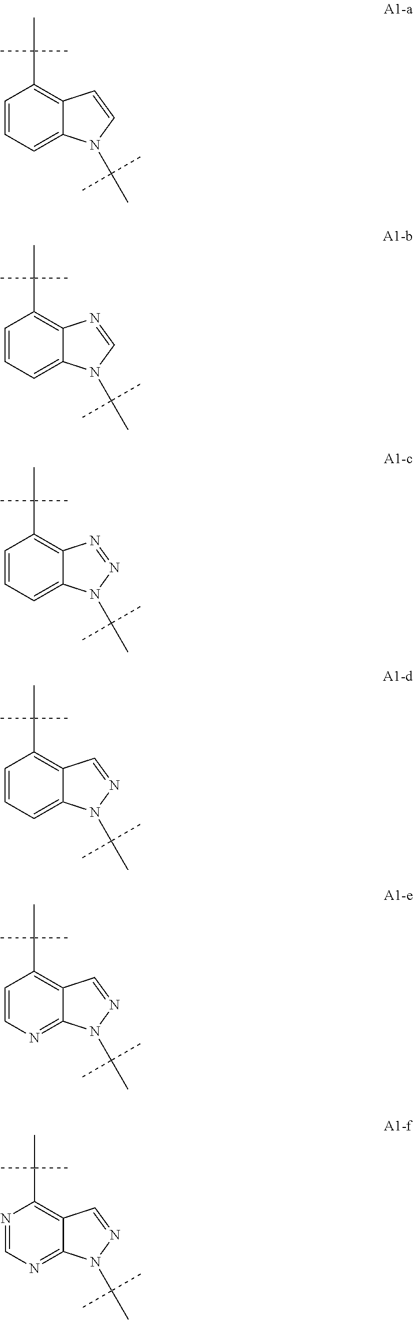 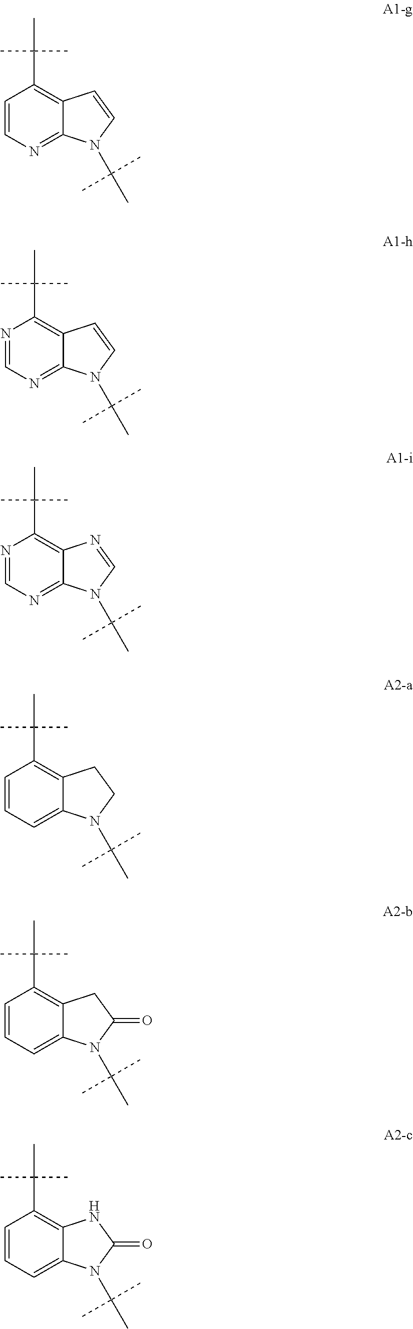 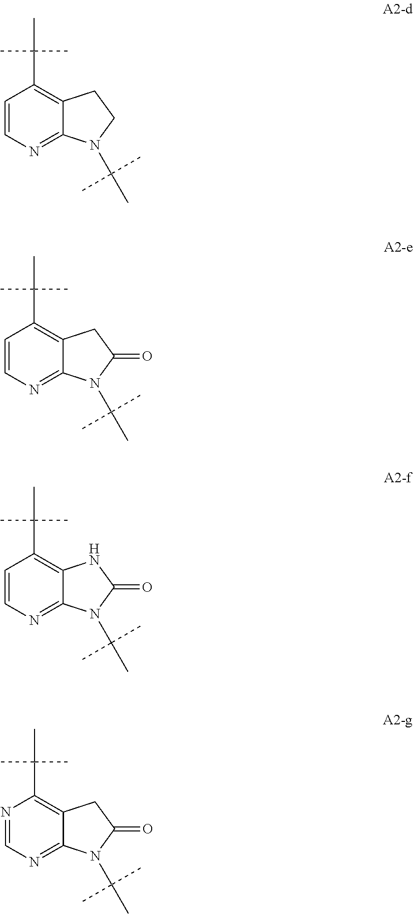
each of which is optionally substituted with one or more independent R⁵.

12. The compound of claim 11, wherein A is A1-a, A1-b, A1-d, A1-e, A1-g, A2-a, A2-b, A2-d, or A2-e, each of which is optionally substituted with one or more independent R⁵.

13. The compound of claim 12, wherein A is A1-a, A1-b, A1-d, A1-g, A2-a, or A2-d, each of which is optionally substituted with one or more independent R⁵.

14. The compound of claim 2, wherein L′ is a covalent bond; Ar is C₆₋₁₂aryl or C₅₋₁₂heteroaryl, each of which is optionally substituted with one or more independent R⁶; L has 1 to 4 carbon atoms and is optionally deuterium-enriched and optionally substituted with one or more independent R³; and A is A1-a, A1-b, A1-c, A1-d, A1-e, A1-f, A1-g, A1-h, A1-i, A2-a, A2-b, A2-c, A2-d, A2-e, A2-f, or A2-g, each of which is optionally substituted with one or more independent R⁵.

15. The compound of claim 14, wherein L has 1 or 2 carbon atoms and is optionally deuterium-enriched and optionally substituted with one or more independent R³; and A is A1-a, A1-b, A1-d, A1-e, A1-g, A2-a, A2-b, A2-d, or A2-e, each of which is optionally substituted with one or more independent R⁵.

16. The compound of claim 15, wherein L is —CD₂-, —CHD-, or —CD₂CD₂-; and A is A1-a, A1-b, A1-d, A1-g, A2-a, or A2-d, each of which is optionally substituted with one or more independent R⁵.

17. The compound of claim 15, wherein L is —CH₂—; Ar is C₆₋₁₂aryl or C₅₋₁₂heteroaryl and is substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

18. The compound of claim 1, wherein E is E1 or E2; Y is N; and X, Z, V, and U are each independently C—R¹:

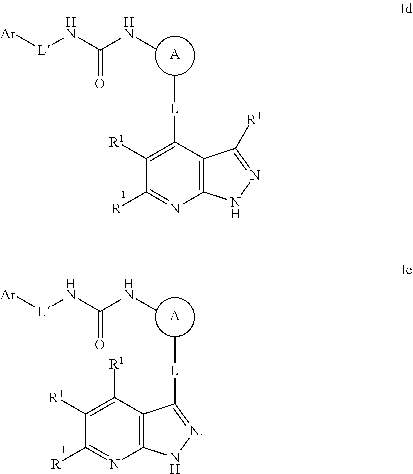

19. The compound of claim 18, wherein L′ is a covalent bond.

20. The compound of claim 18, wherein Ar is C₆₋₁₂aryl or C₅₋₁₂heteroaryl, each of which is optionally substituted with one or more independent R⁶.

21. The compound of claim 20, wherein Ar is substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

22. The compound of claim 18, wherein L has 1 to 4 carbon atoms and is optionally deuterium-enriched and optionally substituted with one or more independent R³.

23. The compound of claim 22, wherein L has 1 or 2 carbon atoms and is optionally deuterium-enriched and optionally substituted with one or more independent R³.

24. The compound of claim 23, wherein L has one carbon atom and is optionally deuterium-enriched and optionally substituted with one or more independent R³.

25. The compound of claim 23, wherein L is —CD₂-, —CHD-, or —CD₂CD₂-.

26. The compound of claim 23, wherein L is —CH₂—; Ar is C₆₋₁₂aryl or C₅₋₁₂heteroaryl and substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

27. The compound of claim 18, wherein A is selected from A1-a, A1-b, A1-c, A1-d, A1-e, A1-f, A1-g, A1-h, A1-i, A2-a, A2-b, A2-c, A2-d, A2-e, A2-f, or A2-g: 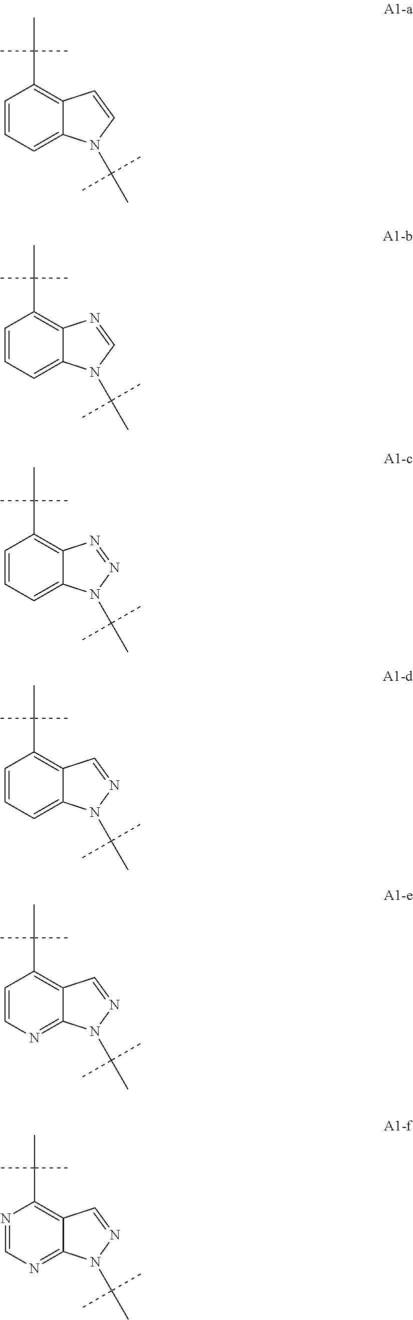 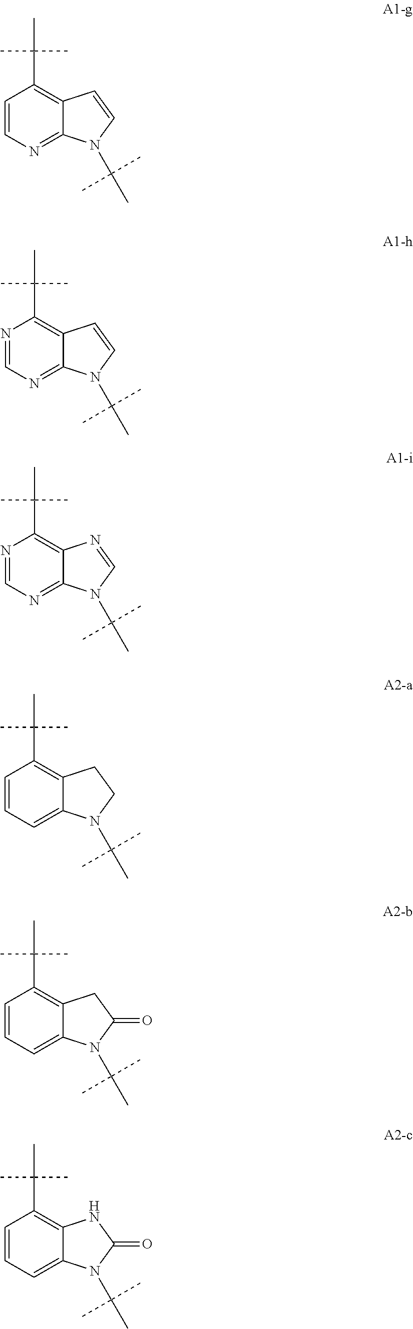 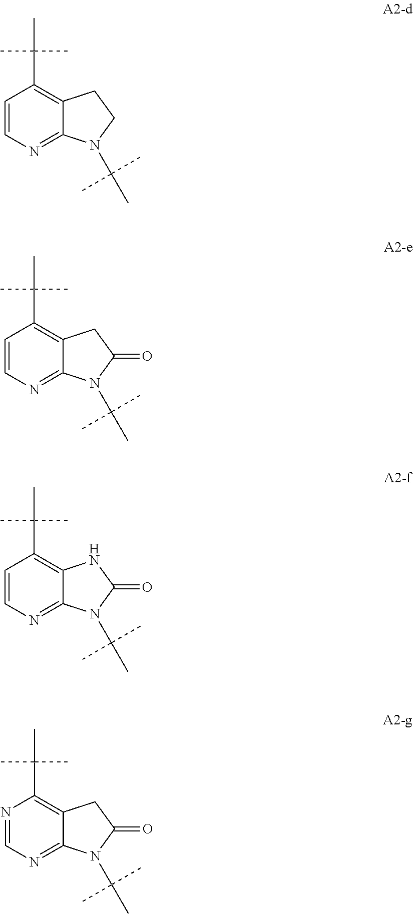
each of which is optionally substituted with one or more independent R⁵.

28. The compound of claim 27, wherein A is A1-a, A1-b, A1-d, A1-e, A1-g, A2-a, A2-b, A2-d, or A2-e, each of which is optionally substituted with one or more independent R⁵.

29. The compound of claim 28, wherein A is A1-a, A 1-b, A1-d, A1-g, A2-a, or A2-d, each of which is optionally substituted with one or more independent R⁵.

30. The compound of claim 18, wherein L′ is a covalent bond; Ar is C₆₋₁₂aryl or C₅₋₁₂heteroaryl, each of which is optionally substituted with one or more independent Rb; L has 1 to 4 carbon atoms and is optionally deuterium-enriched and optionally substituted with one or more independent R³; and A is A1-a, A1-b, A1-c, A1-d, A1-e, A1-f, A1-g, A1-h, A1-i, A2-a, A2-b, A2-c, A2-d, A2-e, A2-f, or A2-g, each of which is optionally substituted with one or more independent R⁵.

31. The compound of claim 30, wherein L has 1 or 2 carbon atoms and is optionally deuterium-enriched and optionally substituted with one or more independent R³; and A is A1-a, A1-b, A1-d, A1-e, A1-g, A2-a, A2-b, A2-d, or A2-e, each of which is optionally substituted with one or more independent R⁵.

32. The compound of claim 31, wherein L is —CD₂-, —CHD-, or —CD₂CD₂-; and A is A1-a, A1-b, A1-d, A1-g, A2-a, or A2-d, each of which is optionally substituted with one or more independent R⁵.

33. The compound of claim 31, wherein L is —CH₂—; Ar is C₆₋₁₂aryl or C₅₋₁₂heteroaryl and substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

34. The compound of claim 1, wherein Z is N; and X, Y, V, and U are each independently C—R¹.

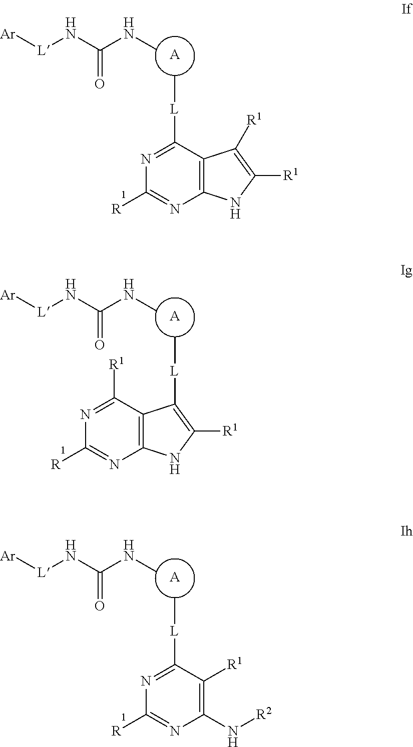

35. The compound of claim 34, wherein L′ is a covalent bond.

36. The compound of claim 34, wherein Ar is C₆₋₁₂aryl or C₅₋₁₂heteroaryl, each of which is optionally substituted with one or more independent R⁶.

37. The compound of claim 36, wherein Ar is substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

38. The compound of claim 34, wherein L has 1 to 4 carbon atoms and is optionally deuterium-enriched and optionally substituted with one or more independent R³.

39. The compound of claim 38, wherein L has 1 or 2 carbon atoms and is optionally deuterium-enriched and optionally substituted with one or more independent R³.

40. The compound of claim 39, wherein L has one carbon atom and is optionally deuterium-enriched and optionally substituted with one or more independent R³.

41. The compound of claim 39, wherein L is —CD₂-, —CHD-, or —CD₂CD₂-.

42. The compound of claim 39, wherein L is —CH₂—; Ar is C₆₋₁₂aryl or C₅₋₁₂heteroaryl and substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

43. The compound of claim 34, wherein A is A1-a, A 1-b, A1-c, A1-d, A1-e, A1-f, A1-g, A1-h, A1-i, A2-a, A2-b, A2-c, A2-d, A2-e, A2-f, or A2-g: 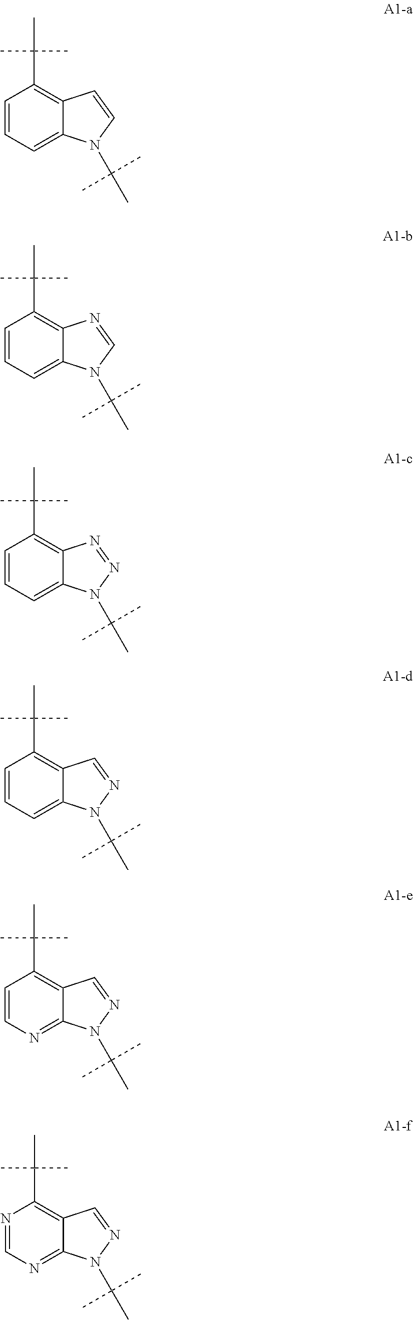 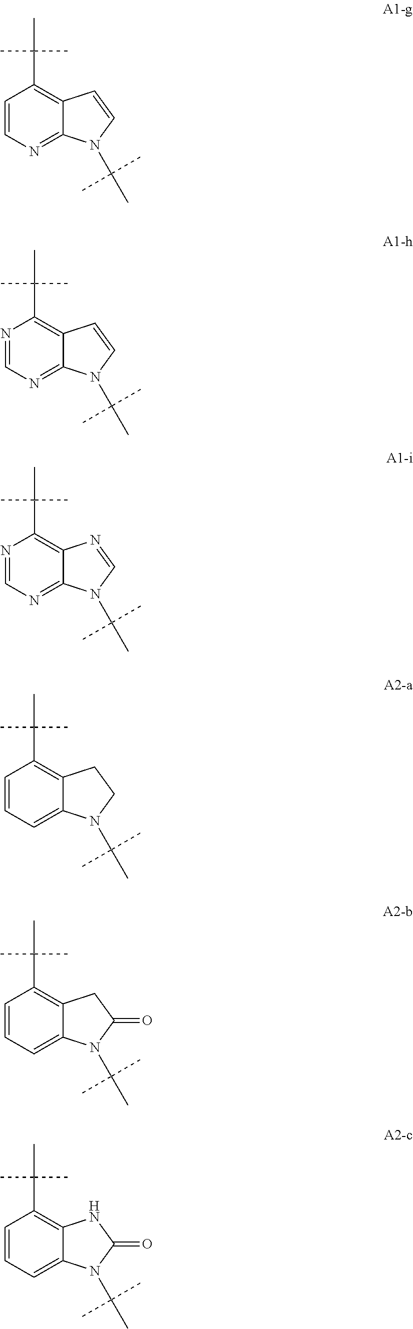 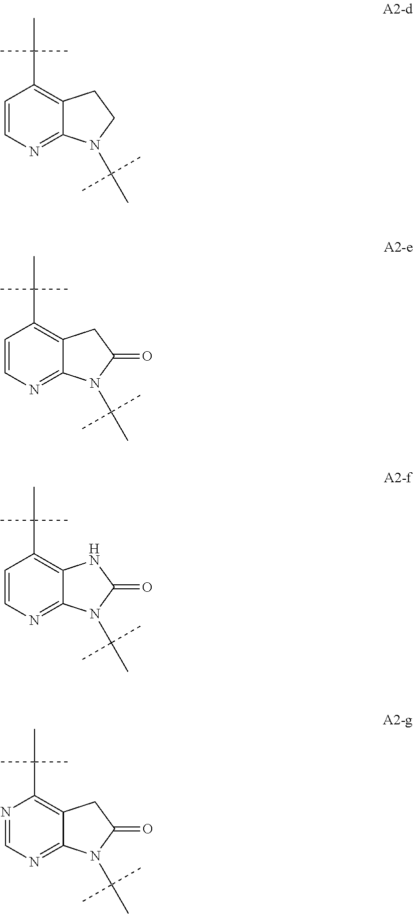
each of which is optionally substituted with one or more independent R⁵.

44. The compound of claim 43, wherein A is A1-a, A1-b, A1-d, A1-e, A1-g, A2-a, A2-b, A2-d, or A2-e, each of which is optionally substituted with one or more independent R⁵.

45. The compound of claim 44, wherein A is A1-a, A1-b, A1-d, A1-g, A2-a, or A2-d, each of which is optionally substituted with one or more independent R⁵.

46. The compound of claim 34, wherein L′ is a covalent bond; Ar is C₆₋₁₂aryl or C₅₋₁₂heteroaryl, each of which is optionally substituted with one or more independent R⁶; L has 1 to 4 carbon atoms and is optionally deuterium-enriched and optionally substituted with one or more independent R³; and A is A1-a, A1-b, A1-c, A1-d, A1-e, A1-f, A1-g, A1-h, A1-i, A2-a, A2-b, A2-c, A2-d, A2-e, A2-f, or A2-g, each of which is optionally substituted with one or more independent R⁵.

47. The compound of claim 46, wherein L has 1 or 2 carbon atoms and is optionally deuterium-enriched and optionally substituted with one or more independent R³; and A is A1-a, A1-b, A1-d, A1-e, A1-g, A2-a, A2-b, A2-d, or A2-e, each of which is optionally substituted with one or more independent R⁵.

48. The compound of claim 47, wherein L is —CD₂-, —CHD-, or —CD₂CD₂-; and A is A1-a, A1-b, A1-d, A1-g, A2-a, or A2-d, each of which is optionally substituted with one or more independent R⁵.

49. The compound of claim 47, wherein L is —CH₂—; Ar is C₆₋₁₂aryl or C₅₋₁₂heteroaryl and substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

50. The compound of claim 1, wherein X is N; and Y, Z, and V are each independently C—R¹:

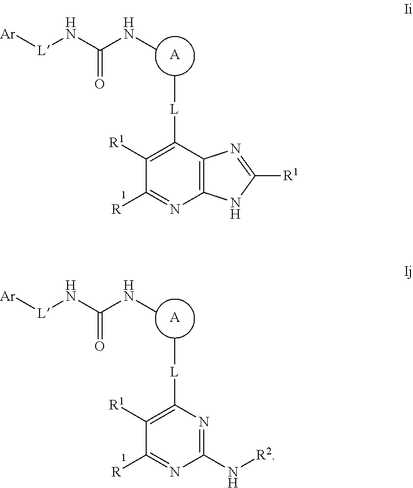

51. The compound of claim 50, wherein L′ is a covalent bond.

52. The compound of claim 50, wherein Ar is C₆₋₁₂aryl or C₅₋₁₂heteroaryl, each of which is optionally substituted with one or more independent R⁶.

53. The compound of claim 50, wherein Ar is substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

54. The compound of claim 50, wherein L has 1 to 4 carbon atoms and is optionally deuterium-enriched and optionally substituted with one or more independent R³.

55. The compound of claim 54, wherein L has 1 or 2 carbon atoms and is optionally deuterium-enriched and optionally substituted with one or more independent R³.

56. The compound of claim 55, wherein L has one carbon atom and is optionally deuterium-enriched and optionally substituted with one or more independent R³.

57. The compound of claim 55, wherein L is —CD₂-, —CHD-, or —CD₂CD₂-.

58. The compound of claim 55, wherein L is —CH₂—; Ar is C₆₋₁₂aryl or C₅₋₁₂heteroaryl and substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

59. The compound of claim 50, wherein A is A1-a, A1-b, A1-c, A1-d, A1-e, A1-f, A1-g, A1-h, A1-i, A2-a, A2-b, A2-c, A2-d, A2-e, A2-f, or A2-g: 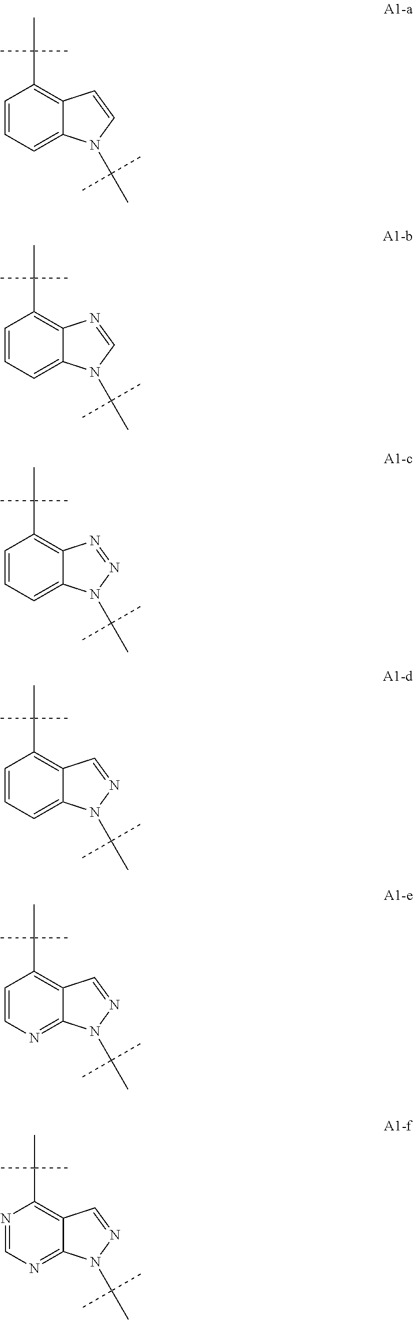 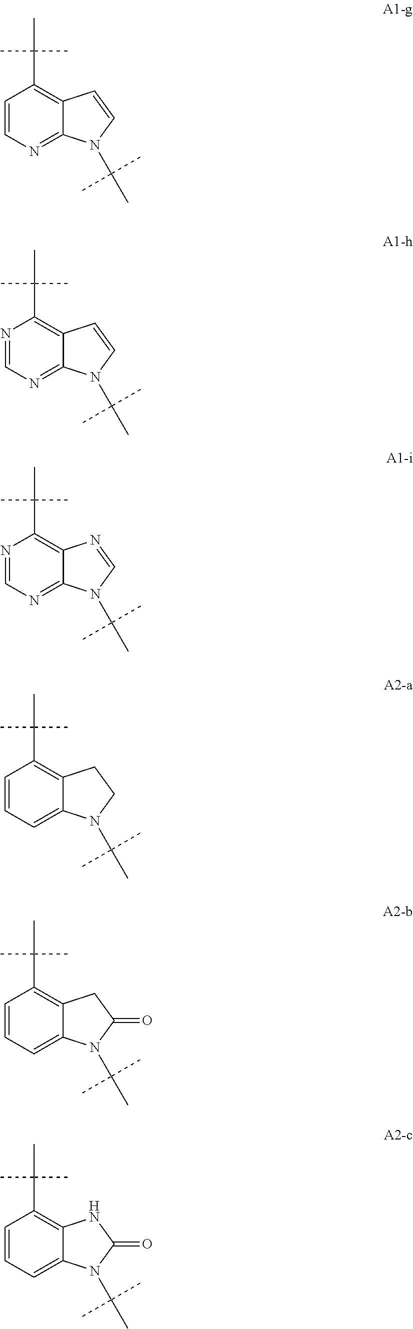 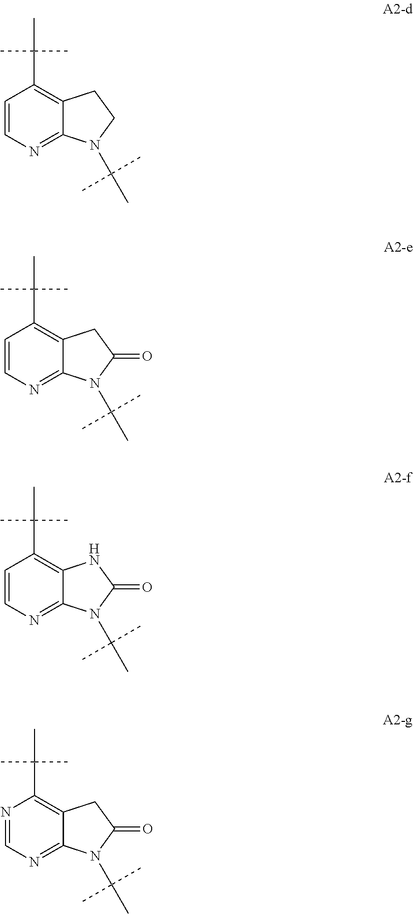
each of which is optionally substituted with one or more independent R⁵.

60. The compound of claim 59, wherein A is A1-a, A1-b, A1-d, A1-e, A1-g, A2-a, A2-b, A2-d, or A2-e, each of which is optionally substituted with one or more independent R⁵.

61. The compound of claim 60, wherein A is A1-a, A1-b, A1-d, A1-g, A2-a, or A2-d, each of which is optionally substituted with one or more independent R⁵.

62. The compound of claim 50, wherein L′ is a covalent bond; Ar is C₆₋₁₂aryl or C₅₋₁₂heteroaryl, each of which is optionally substituted with one or more independent R⁶; L has 1 to 4 carbon atoms and is optionally deuterium-enriched and optionally substituted with one or more independent R³; and A is A1-a, A1-b, A1-c, A1-d, A1-e, A1-f, A1-g, A1-h, A1-i, A2-a, A2-b, A2-c, A2-d, A2-e, A2-f, or A2-g, each of which is optionally substituted with one or more independent R⁵.

63. The compound of claim 62, wherein L has 1 or 2 carbon atoms and is optionally deuterium-enriched and optionally substituted with one or more independent R³; and A is A1-a, A1-b, A1-d, A1-e, A1-g, A2-a, A2-b, A2-d, or A2-e, each of which is optionally substituted with one or more independent R⁵.

64. The compound of claim 63, wherein L is —CD₂-, —CHD-, or —CD₂CD₂-; and A is A1-a, A1-b, A1-d, A1-g, A2-a, or A2-d, each of which is optionally substituted with one or more independent R⁵.

65. The compound of claim 63, wherein L is —CH₂—; Ar is C₆₋₁₂aryl or C₅₋₁₂heteroaryl and substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

66. The compound of claim 1, wherein X and Z are N; and Y and V are each independently C—R¹.

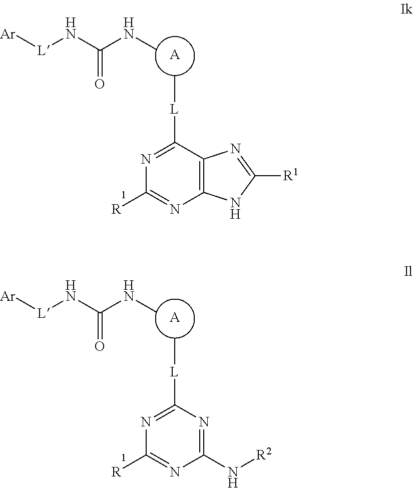

67. The compound of claim 66, wherein L′ is a covalent bond.

68. The compound of claim 66, wherein Ar is C₆₋₁₂aryl or C₅₋₁₂heteroaryl, each of which is optionally substituted with one or more independent R⁶.

69. The compound of claim 66, wherein L has 1 to 4 carbon atoms and is optionally deuterium-enriched and optionally substituted with one or more independent R³.

70. The compound of claim 69, wherein L has 1 or 2 carbon atoms and is optionally deuterium-enriched and optionally substituted with one or more independent R³.

71. The compound of claim 70, wherein L has one carbon atom and is optionally deuterium-enriched and optionally substituted with one or more independent R³.

72. The compound of claim 70, wherein L is —CD₂-, —CHD-, or —CD₂CD₂-.

73. The compound of claim 69, wherein L is —CH₂—, Ar is C₆₋₁₂aryl or C₅₋₁₂heteroaryl and substituted with one or more independent R⁶, and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

74. The compound of claim 66, wherein A is A1-a, A 1-b, A1-c, A1-d, A1-e, A1-f, A1-g, A1-h, A1-i, A2-a, A2-b, A2-c, A2-d, A2-e, A2-f, or A2-g: 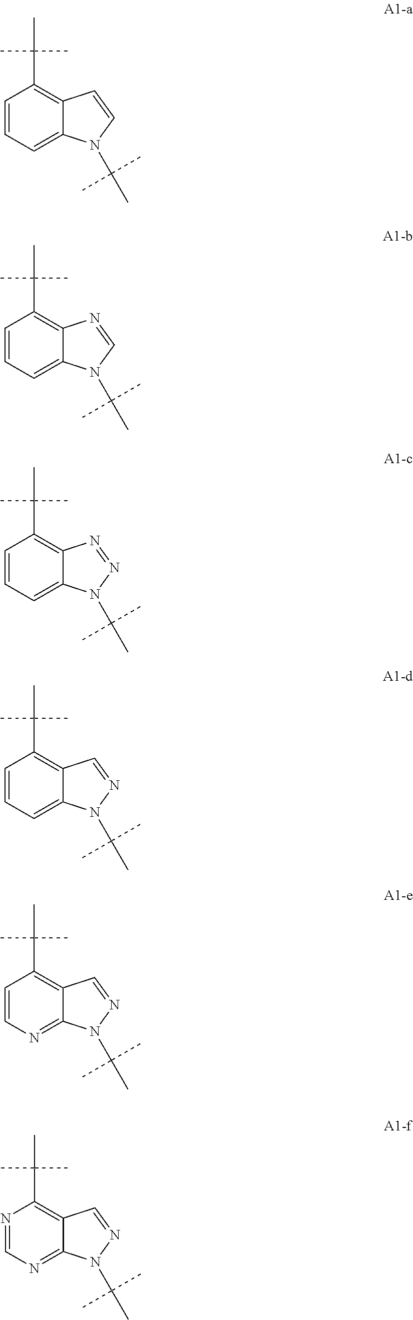 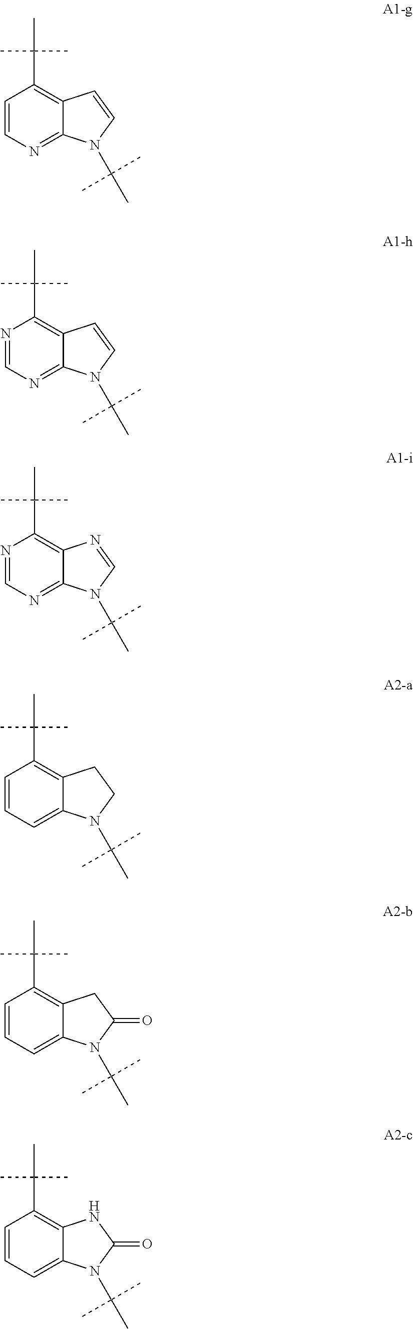 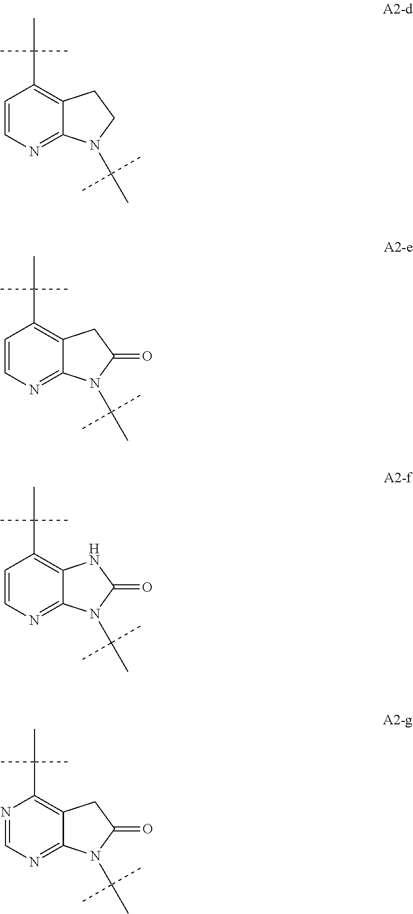
each of which is optionally substituted with one or more independent R⁵.

75. The compound of claim 74, wherein A is A1-a, A1-b, A1-d, A1-e, A1-g, A2-a, A2-b, A2-d, or A2-e, each of which is optionally substituted with one or more independent R⁵.

76. The compound of claim 75, wherein A is A1-a, A1-b, A1-d, A1-g, A2-a, or A2-d, each of which is optionally substituted with one or more independent R⁵.

77. The compound of claim 66, wherein L′ is a covalent bond; Ar is C₆₋₁₂aryl or C₅₋₁₂heteroaryl, each of which is optionally substituted with one or more independent R⁶; L has 1 to 4 carbon atoms and is optionally deuterium-enriched and optionally substituted with one or more independent R³; and A is A1-a, A1-b, A1-c, A1-d, A1-e, A1-f, A1-g, A1-h, A1-i, A2-a, A2-b, A2-c, A2-d, A2-e, A2-f, or A2-g, each of which is optionally substituted with one or more independent R⁵.

78. The compound of claim 77, wherein L has 1 or 2 carbon atoms and is optionally deuterium-enriched and optionally substituted with one or more independent R³; and A is A1-a, A1-b, A1-d, A1-e, A1-g, A2-a, A2-b, A2-d, or A2-e, each of which is optionally substituted with one or more independent R⁵.

79. The compound of claim 77, wherein L is —CD₂-, —CHD-, or —CD₂CD₂-; and A is A1-a, A1-b, A1-d, A1-g, A2-a, or A2-d, each of which is optionally substituted with one or more independent R⁵.

80. The compound of claim 78, wherein L is —CH₂—; Ar is C₆₋₁₂aryl or C₅₋₁₂heteroaryl and substituted with one or more independent R⁶; and at least one R⁶ is deuterium-enriched C₁₋₈alkyl, deuterium-enriched C₃₋₁₂cycloalkyl, deuterium-enriched C₃₋₁₂heterocycloalkyl, deuterium-enriched C₁₋₈alkyl-O—, deuterium-enriched C₃₋₁₂cycloalkyl-O—, or deuterium-enriched C₃₋₁₂heterocycloalkyl-O—, each of which is optionally substituted with one or more independent Q².

81. The compound of claim 1, wherein the compound is:
1-[1-[dideuterio(1H-pyrrolo[2,3-b]pyridin-3-yl)methyl]indol-4-yl]-3-phenylurea;
1-[1-[dideuterio(1H-pyrrolo[2,3-b]pyridin-3-yl)methyl]indol-4-yl]-3-(2-fluorophenyl)urea;
1-[1-[dideuterio(1H-pyrrolo[2,3-b]pyridin-3-yl)methyl]indol-4-yl]-3-(3-fluorophenyl)urea;
1-[1-[dideuterio(1H-pyrrolo[2,3-b]pyridin-3-yl)methyl]indol-4-yl]-3-(4-fluorophenyl)urea;
1-[1-[dideuterio(1H-pyrrolo[2,3-b]pyridin-3-yl)methyl]indol-4-yl]-3-(2-chlorophenyl)urea;
1-[1-[dideuterio(1H-pyrrolo[2,3-b]pyridin-3-yl)methyl]indol-4-yl]-3-(3-chlorophenyl)urea,
1-[1-[dideuterio(1H-pyrrolo[2,3-b]pyridin-3-yl)methyl]indol-4-yl]-3-(4-chlorophenyl)urea;
1-[1-[dideuterio(1H-pyrrolo[2,3-b]pyridin-3-yl)methyl]indol-4-yl]-3-(2-bromophenyl)urea;
1-[1-[dideuterio(1H-pyrrolo[2,3-b]pyridin-3-yl)methyl]indol-4-yl]-3-(3-bromophenyl)urea;
1-[1-[dideuterio(1H-pyrrolo[2,3-b]pyridin-3-yl)methyl]indol-4-yl]-3-(4-bromophenyl)urea;
1-[1-[dideuterio(1H-pyrrolo[2,3-b]pyridin-3-yl)methyl]indol-4-yl]-3-(2-methylphenyl)urea;
1-[1-[dideuterio(1H-pyrrolo[2,3-b]pyridin-3-yl)methyl]indol-4-yl]-3-(3-methylphenyl)urea;
1-[1-[dideuterio(1H-pyrrolo[2,3-b]pyridin-3-yl)methyl]indol-4-yl]-3-(4-methylphenyl)urea;
1-[1-[dideuterio(1H-pyrrolo[2,3-b]pyridin-3-yl)methyl]indol-4-yl]-3-(2-methoxyphenyl)urea;
1-[1-[dideuterio(1H-pyrrolo[2,3-b]pyridin-3-yl)methyl]indol-4-yl]-3-(3-methoxyphenyl)urea;
1-[1-[dideuterio(1H-pyrrolo[2,3-b]pyridin-3-yl)methyl]indol-4-yl]-3-(4-methoxyphenyl)urea;
1-[1-[dideuterio(1H-pyrrolo[2,3-b]pyridin-3-yl)methyl]indol-4-yl]-3-(2-trifluoromethoxyphenyl)urea;
1-[1-[dideuterio(1H-pyrrolo[2,3-b]pyridin-3-yl)methyl]indol-4-yl]-3-(3-trifluoromethoxyphenyl)urea;
1-[1-[dideuterio(1H-pyrrolo[2,3-b]pyridin-3-yl)methyl]indol-4-yl]-3-(4-trifluoromethoxyphenyl)urea
1-[1-[dideuterio(1H-pyrrolo[2,3-b]pyridin-3-yl)methyl]indol-4-yl]-3-(2-trifluoromethylphenyl)urea;
1-[1-[dideuterio(1H-pyrrolo[2,3-b]pyridin-3-yl)methyl]indol-4-yl]-3-(3-trifluoromethylphenyl)urea;
1-[1-[dideuterio(1H-pyrrolo[2,3-b]pyridin-3-yl)methyl]indol-4-yl]-3-(4-trifluoromethylphenyl)urea
1-[1-[dideuterio(1H-pyrrolo[2,3-b]pyridin-3-yl)methyl]indol-4-yl]-3-(2-cyanophenyl)urea;
1-[1-[dideuterio(1H-pyrrolo[2,3-b]pyridin-3-yl)methyl]indol-4-yl]-3-(3-cyanophenyl)urea;
1-[1-[dideuterio(1H-pyrrolo[2,3-b]pyridin-3-yl)methyl]indol-4-yl]-3-(4-cyanophenyl)urea;
1-[1-[dideuterio(1H-pyrrolo[2,3-b]pyridin-3-yl)methyl]indol-4-yl]-3-(5-fluoro-2-methoxyphenyl)urea;
1-[1-[dideuterio(1H-pyrrolo[2,3-b]pyridin-3-yl)methyl]indol-4-yl]-3-(5-chloro-2-methoxyphenyl)urea;
1-[1-[dideuterio(1H-pyrrolo[2,3-b]pyridin-3-yl)methy]indol-4-yl]-3-(5-bromo-2-methoxyphenyl)urea;
1-[1-[dideuterio(1H-pyrrolo[2,3-b]pyridin-3-yl)methyl]indol-4-yl]-3-(5-methyl-2-methoxyphenyl)urea;
1-[1-[dideuterio(1H-pyrrolo[2,3-b]pyridin-3-yl)methyl]indol-4-yl]-3-(2,5-dimethoxyphenyl)urea;
1-[1-[dideuterio(1H-pyrrolo[2,3-b]pyridin-3-yl)methyl]indol-4-yl]-3-(5-trifluoromethyl-2-methoxyphenyl)urea;
1-[1-[dideuterio(1H-pyrrolo[2,3-b]pyridin-3-yl)methyl]indol-4-yl]-3-(5-methyl-2-fluorophenyl)urea;
1-[1-[dideuterio(1H-pyrrolo[2,3-b]pyridin-3-yl)methyl]indol-4-yl]-3-(5-methoxyl-2-fluorophenyl)urea;
1-[1-[dideuterio(1H-pyrrolo[2,3-b]pyridin-3-yl)methyl]indol-4-yl]-3-(5-trifluoromethyl-2-fluorophenyl)urea;
1-[1-[dideuterio(1H-pyrrolo[2,3-b]pyridin-3-yl)methyl]indol-4-yl]-3-(5-chloro-2-fluorophenyl)urea;
1-[1-[dideuterio(1H-pyrrolo[2,3-b]pyridin-3-yl)methyl]indol-4-yl]-3-(5-bromo-2-fluorophenyl)urea; or
1-[1-[dideuterio(1H-pyrrolo[2,3-b]pyridin-3-yl)methyl]indol-4-yl]-3-(5-methyl-2-trifluoromethylphenyl)urea.

82. The compound of claim 1, wherein the compound is:
1-[1-[dideuterio(1H-pyrrolo[2,3-b]pyridin-4-yl)methyl]indol-4-yl]-3-phenylurea;
1-[1-[dideuterio(1H-pyrrolo[2,3-b]pyridin-4-yl)methyl]indol-4-yl]-3-(2-fluorophenyl)urea;
1-[1-[dideuterio(1H-pyrrolo[2,3-b]pyridin-4-yl)methyl]indol-4-yl]-3-(3-fluorophenyl)urea;
1-[1-[dideuterio(1H-pyrrolo[2,3-b]pyridin-4-yl)methyl]indol-4-yl]-3-(4-fluorophenyl)urea;
1-[1-[dideuterio(1H-pyrrolo[2,3-b]pyridin-4-yl)methyl]indol-4-yl]-3-(2-chlorophenyl)urea;
1-[1-[dideuterio(1H-pyrrolo[2,3-b]pyridin-4-yl)methyl]indol-4-yl]-3-(3-chlorophenyl)urea;
1-[1-[dideuterio(1H-pyrrolo[2,3-b]pyridin-4-yl)methyl]indol-4-yl]-3-(4-chlorophenyl)urea;
1-[1-[dideuterio(1H-pyrrolo[2,3-b]pyridin-4-yl)methyl]indol-4-yl]-3-(2-bromophenyl)urea;
1-[1-[dideuterio(1H-pyrrolo[2,3-b]pyridin-4-yl)methyl]indol-4-yl]-3-(3-bromophenyl)urea;
1-[1-[dideuterio(1H-pyrrolo[2,3-b]pyridin-4-yl)methyl]indol-4-yl]-3-(4-bromophenyl)urea;
1-[1-[dideuterio(1H-pyrrolo[2,3-b]pyridin-4-yl)methyl]indol-4-yl]-3-(2-methylphenyl)urea;
1-[1-[dideuterio(1H-pyrrolo[2,3-b]pyridin-4-yl)methyl]indol-4-yl]-3-(3-methylphenyl)urea;
1-[1-[dideuterio(1H-pyrrolo[2,3-b]pyridin-4-yl)methyl]indol-4-yl]-3-(4-methylphenyl)urea;
1-[1-[dideuterio(1H-pyrrolo[2,3-b]pyridin-4-yl)methyl]indol-4-yl]-3-(2-methoxyphenyl)urea;
1-[1-[dideuterio(1H-pyrrolo[2,3-b]pyridin-4-yl)methyl]indol-4-yl]-3-(3-methoxyphenyl)urea;
1-[1-[dideuterio(1H-pyrrolo[2,3-b]pyridin-4-yl)methyl]indol-4-yl]-3-(4-methoxyphenyl)urea;
1-[1-[dideuterio(1H-pyrrolo[2,3-b]pyridin-4-yl)methyl]indol-4-yl]-3-(2-trifluoromethoxyphenyl)urea;
1-[1-[dideuterio(1H-pyrrolo[2,3-b]pyridin-4-yl)methyl]indol-4-yl]-3-(3-trifluoromethoxyphenyl)urea;
1-[1-[dideuterio(1H-pyrrolo[2,3-b]pyridin-4-yl)methyl]indol-4-yl]-3-(4-trifluoromethoxyphenyl)urea;
1-[1-[dideuterio(1H-pyrrolo[2,3-b]pyridin-4-yl)methyl]indol-4-yl]-3-(2-trifluoromethylphenyl)urea;
1-[1-[dideuterio(1H-pyrrolo[2,3-b]pyridin-4-yl)methyl]indol-4-yl]-3-(3-trifluoromethylphenyl)urea;
1-[1-[dideuterio(1H-pyrrolo[2,3-b]pyridin-4-yl)methyl]indol-4-yl]-3-(4-trifluoromethylphenyl)urea;
1-[1-[dideuterio(1H-pyrrolo[2,3-b]pyridin-4-yl)methyl]indol-4-yl]-3-(2-cyanophenyl)urea;
1-[1-[dideuterio(1H-pyrrolo[2,3-b]pyridin-4-yl)methyl]indol-4-yl]-3-(3-cyanophenyl)urea;
1-[1-[dideuterio(1H-pyrrolo[2,3-b]pyridin-4-yl)methyl]indol-4-yl]-3-(4-cyanophenyl)urea;
1-[1-[dideuterio(1H-pyrrolo[2,3-b]pyridin-4-yl)methyl]indol-4-yl]-3-(5-fluoro-2-methoxyphenyl)urea;
1-[1-[dideuterio(1H-pyrrolo[2,3-b]pyridin-4-yl)methyl]indol-4-yl]-3-(5-chloro-2-methoxyphenyl)urea;
1-[5-chloro-2-(trideuteriomethoxy)phenyl]-3-[1-[dideuterio(1H-pyrrolo[2,3-b]pyridin-4-yl)methyl]indol-4-yl]urea hydrochloride;
1-[1-[dideuterio(1H-pyrrolo[2,3-b]pyridin-4-yl)methyl]indol-4-yl]-3-(5-bromo-2-methoxyphenyl)urea;
1-[1-[dideuterio(1H-pyrrolo[2,3-b]pyridin-4-yl)methyl]indol-4-yl]-3-(5-methyl-2-methoxyphenyl)urea;
1-[1-[dideuterio(1H-pyrrolo[2,3-b]pyridin-4-yl)methyl]indol-4-yl]-3-(2,5-dimethoxyphenyl)urea;
1-[1-[dideuterio(1H-pyrrolo[2,3-b]pyridin-4-yl)methyl]indol-4-yl]-3-(5-trifluoromethyl-2-methoxyphenyl)urea;
1-[1-[dideuterio(1H-pyrrolo[2,3-b]pyridin-4-yl)methyl]indol-4-yl]-3-(5-methyl-2-fluorophenyl)urea;
1-[1-[dideuterio(1H-pyrrolo[2,3-b]pyridin-4-yl)methyl]indol-4-yl]-3-(5-methoxyl-2-fluorophenyl)urea;
1-[1-[dideuterio(1H-pyrrolo[2,3-b]pyridin-4-yl)methyl]indol-4-yl]-3-(5-trifluoromethyl-2-fluorophenyl)urea;
1-[1-[dideuterio(1H-pyrrolo[2,3-b]pyridin-4-yl)methyl]indol-4-yl]-3-(5-chloro-2-fluorophenyl)urea;
1-[1-[dideuterio(1H-pyrrolo[2,3-b]pyridin-4-yl)methyl]indol-4-yl]-3-(5-bromo-2-fluorophenyl)urea; or
1-[1-[dideuterio(1H-pyrrolo[2,3-b]pyridin-4-yl)methyl]indol-4-yl]-3-(5-methyl-2-trifluoromethylphenyl)urea.

83. The compound of claim 1, wherein the compound is:
1-[1-[dideuterio(1H-pyrrolo[2,3-b]pyridin-3-yl)methyl]indolin-4-yl]-3-phenylurea;
1-[1-[dideuterio(1H-pyrrolo[2,3-b]pyridin-3-yl)methyl]indolin-4-yl]-3-(2-fluorophenyl)urea;
1-[1-[dideuterio(1H-pyrrolo[2,3-b]pyridin-3-yl)methyl]indolin-4-yl]-3-(3-fluorophenyl)urea;
1-[1-[dideuterio(1H-pyrrolo[2,3-b]pyridin-3-yl)methyl]indolin-4-yl]-3-(4-fluorophenyl)urea;
1-[1-[dideuterio(1H-pyrrolo[2,3-b]pyridin-3-yl)methyl]indolin-4-yl]-3-(2-chlorophenyl)urea;
1-[1-[dideuterio(1H-pyrrolo[2,3-b]pyridin-3-yl)methyl]indolin-4-yl]-3-(3-chlorophenyl)urea;
1-[1-[dideuterio(1H-pyrrolo[2,3-b]pyridin-3-yl)methyl]indolin-4-yl]-3-(4-chlorophenyl)urea;
1-[1-[dideuterio(1H-pyrrolo[2,3-b]pyridin-3-yl)methyl]indolin-4-yl]-3-(2-bromophenyl)urea;
1-[1-[dideuterio(1H-pyrrolo[2,3-b]pyridin-3-yl)methyl]indolin-4-yl]-3-(3-bromophenyl)urea;
1-[1-[dideuterio(1H-pyrrolo[2,3-b]pyridin-3-yl)methyl]indolin-4-yl]-3-(4-bromophenyl)urea;
1-[1-[dideuterio(1H-pyrrolo[2,3-b]pyridin-3-yl)methyl]indolin-4-yl]-3-(2-methylphenyl)urea;
1-[1-[dideuterio(1H-pyrrolo[2,3-b]pyridin-3-yl)methyl]indolin-4-yl]-3-(3-methylphenyl)urea;
1-[1-[dideuterio(1H-pyrrolo[2,3-b]pyridin-3-yl)methyl]indolin-4-yl]-3-(4-methylphenyl)urea;
1-[1-[dideuterio(1H-pyrrolo[2,3-b]pyridin-3-yl)methyl]indolin-4-yl]-3-(2-methoxyphenyl)urea;
1-[1-[dideuterio(1H-pyrrolo[2,3-b]pyridin-3-yl)methyl]indolin-4-yl]-3-(3-methoxyphenyl)urea;
1-[1-[dideuterio(1H-pyrrolo[2,3-b]pyridin-3-yl)methyl]indolin-4-yl]-3-(4-methoxyphenyl)urea;
1-[1-[dideuterio(1H-pyrrolo[2,3-b]pyridin-3-yl)methyl]indolin-4-yl]-3-(2-trifluoromethoxyphenyl)urea;
1-[1-[dideuterio(1H-pyrrolo[2,3-b]pyridin-3-yl)methyl]indolin-4-yl]-3-(3-trifluoromethoxyphenyl)urea;
1-[1-[dideuterio(1H-pyrrolo[2,3-b]pyridin-3-yl)methyl]indolin-4-yl]-3-(4-trifluoromethoxyphenyl)urea
1-[1-[dideuterio(1H-pyrrolo[2,3-b]pyridin-3-yl)methyl]indolin-4-yl]-3-(2-trifluoromethylphenyl)urea;
1-[1-[dideuterio(1H-pyrrolo[2,3-b]pyridin-3-yl)methyl]indolin-4-yl]-3-(3-trifluoromethylphenyl)urea;
1-[1-[dideuterio(1H-pyrrolo[2,3-b]pyridin-3-yl)methyl]indolin-4-yl]-3-(4-trifluoromethylphenyl)urea
1-[1-[dideuterio(1H-pyrrolo[2,3-b]pyridin-3-yl)methyl]indolin-4-yl]-3-(2-cyanophenyl)urea;
1-[1-[dideuterio(1H-pyrrolo[2,3-b]pyridin-3-yl)methyl]indolin-4-yl]-3-(3-cyanophenyl)urea;
1-[1-[dideuterio(1H-pyrrolo[2,3-b]pyridin-3-yl)methyl]indolin-4-yl]-3-(4-cyanophenyl)urea;
1-[1-[dideuterio(1H-pyrrolo[2,3-b]pyridin-3-yl)methyl]indolin-4-yl]-3-(5-fluoro-2-methoxyphenyl)urea;
1-[1-[dideuterio(1H-pyrrolo[2,3-b]pyridin-3-yl)methyl]indolin-4-yl]-3-(5-chloro-2-methoxyphenyl)urea;
1-[1-[dideuterio(1H-pyrrolo[2,3-b]pyridin-3-yl)methyl]indolin-4-yl]-3-(5-bromo-2-methoxyphenyl)urea,
1-[1-[dideuterio(1H-pyrrolo[2,3-b]pyridin-3-yl)methyl]indolin-4-yl]-3-(5-methyl-2-methoxyphenyl)urea;
1-[1-[dideuterio(1H-pyrrolo[2,3-b]pyridin-3-yl)methyl]indolin-4-yl]-3-(2,5-dimethoxyphenyl)urea;
1-[1-[dideuterio(1H-pyrrolo[2,3-b]pyridin-3-yl)methyl]indolin-4-yl]-3-(5-trifluoromethyl-2-methoxyphenyl)urea;
1-[1-[dideuterio(1H-pyrrolo[2,3-b]pyridin-3-yl)methyl]indolin-4-yl]-3-(5-methyl-2-fluorophenyl)urea;
1-[1-[dideuterio(1H-pyrrolo[2,3-b]pyridin-3-yl)methyl]indolin-4-yl]-3-(5-methoxyl-2-fluorophenyl)urea;
1-[1-[dideuterio(1H-pyrrolo[2,3-b]pyridin-3-yl)methyl]indolin-4-yl]-3-(5-trifluoromethyl-2-fluorophenyl)urea;
1-[1-[dideuterio(1H-pyrrolo[2,3-b]pyridin-3-yl)methyl]indolin-4-yl]-3-(5-chloro-2-fluorophenyl)urea;
1-[1-[dideuterio(1H-pyrrolo[2,3-b]pyridin-3-yl)methyl]indolin-4-yl]-3-(5-bromo-2-fluorophenyl)urea; or
1-[1-[dideuterio(1H-pyrrolo[2,3-b]pyridin-3-yl)methyl]indolin-4-yl]-3-(5-methyl-2-trifluoromethylphenyl)urea.

84. The compound of claim 1, wherein the compound is:
1-[1-[dideuterio(1H-pyrrolo[2,3-b]pyridin-4-yl)methyl]indolin-4-yl]-3-phenylurea;
1-[1-[dideuterio(1H-pyrrolo[2,3-b]pyridin-4-yl)methyl]indolin-4-yl]-3-(2-fluorophenyl)urea;
1-[1-[dideuterio(1H-pyrrolo[2,3-b]pyridin-4-yl)methyl]indolin-4-yl]-3-(3-fluorophenyl)urea:
1-[1-[dideuterio(1H-pyrrolo[2,3-b]pyridin-4-yl)methyl]indolin-4-yl]-3-(4-fluorophenyl)urea;
1-[1-[dideuterio(1H-pyrrolo[2,3-b]pyridin-4-yl)methyl]indolin-4-yl]-3-(2-chlorophenyl)urea;
1-[1-[dideuterio(1H-pyrrolo[2,3-b]pyridin-4-yl)methyl]indolin-4-yl]-3-(3-chlorophenyl)urea;
1-[1-[dideuterio(1H-pyrrolo[2,3-b]pyridin-4-yl)methyl]indolin-4-yl]-3-(4-chlorophenyl)urea;
1-[1-[dideuterio(1H-pyrrolo[2,3-b]pyridin-4-yl)methyl]indolin-4-yl]-3-(2-bromophenyl)urea;
1-[1-[dideuterio(1H-pyrrolo[2,3-b]pyridin-4-yl)methyl]indolin-4-yl]-3-(3-bromophenyl)urea;
1-[1-[dideuterio(1H-pyrrolo[2,3-b]pyridin-4-yl)methyl]indolin-4-yl]-3-(4-bromophenyl)urea;
1-[1-[dideuterio(1H-pyrrolo[2,3-b]pyridin-4-yl)methyl]indolin-4-yl]-3-(2-methylphenyl)urea;
1-[1-[dideuterio(1H-pyrrolo[2,3-b]pyridin-4-yl)methyl]indolin-4-yl]-3-(3-methylphenyl)urea;
1-[1-[dideuterio(1H-pyrrolo[2,3-b]pyridin-4-yl)methyl]indolin-4-yl]-3-(m-tolyl)urea hydrochloride;
1-[1-[dideuterio(1H-pyrrolo[2,3-b]pyridin-4-yl)methyl]indolin-4-yl]-3-(4-methylphenyl)urea;
1-[1-[dideuterio(1H-pyrrolo[2,3-b]pyridin-4-yl)methyl]indolin-4-yl]-3-(2-methoxyphenyl)urea;
1-[1-[dideuterio(1H-pyrrolo[2,3-b]pyridin-4-yl)methyl]indolin-4-yl]-3-(3-methoxyphenyl)urea;
1-[1-[dideuterio(1H-pyrrolo[2,3-b]pyridin-4-yl)methyl]indolin-4-yl]-3-(4-methoxyphenyl)urea;
1-[1-[dideuterio(1H-pyrrolo[2,3-b]pyridin-4-yl)methyl]indolin-4-yl]-3-(2-trifluoromethoxyphenyl)urea;
1-[1-[dideuterio(1H-pyrrolo[2,3-b]pyridin-4-yl)methyl]indolin-4-yl]-3-(3-trifluoromethoxyphenyl)urea;
1-[1-[dideuterio(1H-pyrrolo[2,3-b]pyridin-4-yl)methyl]indolin-4-yl]-3-(4-trifluoromethoxyphenyl)urea;
1-[1-[dideuterio(1H-pyrrolo[2,3-b]pyridin-4-yl)methyl]indolin-4-yl]-3-(2-trifluoromethylphenyl)urea;
1-[1-[dideuterio(1H-pyrrolo[2,3-b]pyridin-4-yl)methyl]indolin-4-yl]-3-(3-trifluoromethylphenyl)urea;
1-[1-[dideuterio(1H-pyrrolo[2,3-b]pyridin-4-yl)methyl]indolin-4-yl]-3-(4-trifluoromethylphenyl)urea;
1-[1-[dideuterio(1H-pyrrolo[2,3-b]pyridin-4-yl)methyl]indolin-4-yl]-3-(2-cyanophenyl)urea;
1-[1-[dideuterio(1H-pyrrolo[2,3-b]pyridin-4-yl)methyl]indolin-4-yl]-3-(3-cyanophenyl)urea;
1-[1-[dideuterio(1H-pyrrolo[2,3-b]pyridin-4-yl)methyl]indolin-4-yl]-3-(4-cyanophenyl)urea;
1-[1-[dideuterio(1H-pyrrolo[2,3-b]pyridin-4-yl)methyl]indolin-4-yl]-3-(5-fluoro-2-methoxyphenyl)urea;
1-[1-[dideuterio(1H-pyrrolo[2,3-b]pyridin-4-yl)methyl]indolin-4-yl]-3-(5-chloro-2-methoxyphenyl)urea;
1-[5-chloro-2-(trideuteriomethoxy)phenyl]-3-[1-[dideuterio(1H-pyrrolo[2,3-b]pyridin-4-yl)methyl]indolin-4-yl]urea hydrochloride;
1-(5-chloro-2-methoxyphenyl)-3-[1-[dideuterio(1H-pyrrolo[2,3-b]pyridin-4-yl)methyl]indolin-4-yl]urea hydrochloride;
1-[1-[dideuterio(1H-pyrrolo[2,3-b]pyridin-4-yl)methyl]indolin-4-yl]-3-(5-bromo-2-methoxyphenyl)urea;
1-[1-[dideuterio(1H-pyrrolo[2,3-b]pyridin-4-yl)methyl]indolin-4-yl]-3-(5-methyl-2-methoxyphenyl)urea;
1-[1-[dideuterio(1H-pyrrolo[2,3-b]pyridin-4-yl)methyl]indolin-4-yl]-3-(2,5-dimethoxyphenyl)urea;
1-[1-[dideuterio(1H-pyrrolo[2,3-b]pyridin-4-yl)methyl]indolin-4-yl]-3-(5-trifluoromethyl-2-methoxyphenyl)urea;
1-[1-[dideuterio(1H-pyrrolo[2,3-b]pyridin-4-yl)methyl]indolin-4-yl]-3-(5-methyl-2-fluorophenyl)urea;
1-[1-[dideuterio(1H-pyrrolo[2,3-b]pyridin-4-yl)methyl]indolin-4-yl]-3-(5-methoxyl-2-fluorophenyl)urea;
1-[1-[dideuterio(1H-pyrrolo[2,3-b]pyridin-4-yl)methyl]indolin-4-yl]-3-(5-trifluoromethyl-2-fluorophenyl)urea;
1-[1-[dideuterio(1H-pyrrolo[2,3-b]pyridin-4-yl)methyl]indolin-4-yl]-3-(5-chloro-2-fluorophenyl)urea;
1-(5-chloro-2-fluorophenyl)-3-[1-[dideuterio(1H-pyrrolo[2,3-b]pyridin-4-yl)methyl]indolin-4-yl]urea hydrochloride;
1-[1-[dideuterio(1H-pyrrolo[2,3-b]pyridin-4-yl)methyl]indolin-4-yl]-3-(5-bromo-2-fluorophenyl)urea; or
1-[1-[dideuterio(1H-pyrrolo[2,3-b]pyridin-3-yl)methyl]indolin-4-yl]-3-(5-methyl-2-trifluoromethylphenyl)urea.

85. The compound of claim 1, wherein the compound is:
1-[1-[(2-amino-4-pyridyl)dideuteriomethyl]indol-4-yl]-3-phenylurea;
1-[1-[(2-amino-4-pyridyl)dideuteriomethyl]indol-4-yl]-3-(2-fluorophenyl)urea;
1-[1-[(2-amino-4-pyridyl)dideuteriomethyl]indol-4-yl]-3-(3-fluorophenyl)urea;
1-[1-[(2-amino-4-pyridyl)dideuteriomethyl]indol-4-yl]-3-(4-fluorophenyl)urea;
1-[1-[(2-amino-4-pyridyl)dideuteriomethyl]indol-4-yl]-3-(2-chlorophenyl)urea;
1-[1-[(2-amino-4-pyridyl)dideuteriomethyl]indol-4-yl]-3-(3-chlorophenyl)urea;
1-[1-[(2-amino-4-pyridyl)dideuteriomethyl]indol-4-yl]-3-(4-chlorophenyl)urea;
1-[1-[(2-amino-4-pyridyl)dideuteriomethyl]indol-4-yl]-3-(2-bromophenyl)urea;
1-[1-[(2-amino-4-pyridyl)dideuteriomethyl]indol-4-yl]-3-(3-bromophenyl)urea;
1-[1-[(2-amino-4-pyridyl)dideuteriomethyl]indol-4-yl]-3-(4-bromophenyl)urea;
1-[1-[(2-amino-4-pyridyl)dideuteriomethyl]indol-4-yl]-3-(2-methylphenyl)urea;
1-[1-[(2-amino-4-pyridyl)dideuteriomethyl]indol-4-yl]-3-(3-methylphenyl)urea;
1-[1-[(2-amino-4-pyridyl)dideuteriomethyl]indol-4-yl]-3-(4-methylphenyl)urea;
1-[1-[(2-amino-4-pyridyl)dideuteriomethyl]indol-4-yl]-3-(2-methoxyphenyl)urea;
1-[1-[(2-amino-4-pyridyl)dideuteriomethyl]indol-4-yl]-3-(3-methoxyphenyl)urea;
1-[1-[(2-amino-4-pyridyl)dideuteriomethyl]indol-4-yl]-3-(4-methoxyphenyl)urea;
1-[1-[(2-amino-4-pyridyl)dideuteriomethyl]indol-4-yl]-3-(2-trifluoromethoxyphenyl)urea;
1-[1-[(2-amino-4-pyridyl)dideuteriomethyl]indol-4-yl]-3-(3-trifluoromethoxyphenyl)urea;
1-[1-[(2-amino-4-pyridyl)dideuteriomethyl]indol-4-yl]-3-(4-trifluoromethoxyphenyl)urea;
1-[1-[(2-amino-4-pyridyl)dideuteriomethyl]indol-4-yl]-3-(2-trifluoromethylphenyl)urea;
1-[1-[(2-amino-4-pyridyl)dideuteriomethyl]indol-4-yl]-3-(3-trifluoromethylphenyl)urea;
1-[1-[(2-amino-4-pyridyl)dideuteriomethyl]indol-4-yl]-3-(4-trifluoromethylphenyl)urea;
1-[1-[(2-amino-4-pyridyl)dideuteriomethyl]indol-4-yl]-3-(2-cyanophenyl)urea;
1-[1-[(2-amino-4-pyridyl)dideuteriomethyl]indol-4-yl]-3-(3-cyanophenyl)urea;
1-[1-[(2-amino-4-pyridyl)dideuteriomethyl]indol-4-yl]-3-(4-cyanophenyl)urea;
1-[1-[(2-amino-4-pyridyl)dideuteriomethyl]indol-4-yl]-3-(5-fluoro-2-methoxyphenyl)urea;
1-[1-[(2-amino-4-pyridyl)dideuteriomethyl]indol-4-yl]-3-(5-chloro-2-methoxyphenyl)urea;
1-[1-[(2-amino-4-pyridyl)-dideuteriomethyl]indol-4-yl]-3-(5-chloro-2-methoxyphenyl)urea hydrochloride;
1-[1-[(2-amino-4-pyridyl)dideuteriomethyl]indol-4-yl]-3-(5-bromo-2-methoxyphenyl)urea;
1-[1-[(2-amino-4-pyridyl)dideuteriomethyl]indol-4-yl]-3-(5-methyl-2-methoxyphenyl)urea;
1-[1-[(2-amino-4-pyridyl)dideuteriomethyl]indol-4-yl]-3-(2,5-dimethoxyphenyl)urea;
1-[1-[(2-amino-4-pyridyl)dideuteriomethyl]indol-4-yl]-3-(5-trifluoromethyl-2-methoxyphenyl)urea;
1-[1-[(2-amino-4-pyridyl)dideuteriomethyl]indol-4-yl]-3-(5-methyl-2-fluorophenyl)urea;
1-[1-[(2-amino-4-pyridyl)dideuteriomethyl]indol-4-yl]-3-(5-methoxyl-2-fluorophenyl)urea,
1-[1-[(2-amino-4-pyridyl)dideuteriomethyl]indol-4-yl]-3-(5-trifluoromethyl-2-fluorophenyl)urea;
1-[1-[(2-amino-4-pyridyl)dideuteriomethyl]indol-4-yl]-3-(5-chloro-2-fluorophenyl)urea;
1-[1-[(2-amino-4-pyridyl)dideuteriomethyl]indol-4-yl]-3-(5-bromo-2-fluorophenyl)urea;
or
1-[1-[(2-amino-4-pyridyl)dideuteriomethyl]indol-4-yl]-3-(5-methyl-2-trifluoromethylphenyl)urea.

86. The compound of claim 1, wherein the compound is:
1-[1-[dideuterio(1H-pyrrolo[2,3-b]pyridin-3-yl)methyl]indol-4-yl]-3-[2-(trideuteriomethoxy)phenyl]urea;
1-[1-[dideuterio(1H-pyrrolo[2,3-b]pyridin-3-yl)methyl]indol-4-yl]-3-[3-(trideuteriomethoxy)phenyl]urea;
1-[1-[dideuterio(1H-pyrrolo[2,3-b]pyridin-3-yl)methyl]indol-4-yl]-3-[4-(trideuteriomethoxy)phenyl]urea;
1-[1-[dideuterio(1H-pyrrolo[2,3-b]pyridin-3-yl)methyl]indol-4-yl]-3-[5-fluoro-2-(trideuteriomethoxy)phenyl]urea;
1-[1-[dideuterio(1H-pyrrolo[2,3-b]pyridin-3-yl)methyl]indol-4-yl]-3-[5-chloro-2-(trideuteriomethoxy)phenyl]urea;
1-[1-[dideuterio(1H-pyrrolo[2,3-b]pyridin-3-yl)methyl]indol-4-yl]-3-[5-bromo-2-(trideuteriomethoxy)phenyl]urea;
1-[1-[dideuterio(1H-pyrrolo[2,3-b]pyridin-3-yl)methyl]indol-4-yl]-3-[5-methyl-2-(trideuteriomethoxy)phenyl]urea;
1-[1-[dideuterio(1H-pyrrolo[2,3-b]pyridin-3-yl)methyl]indol-4-yl]-3-[2,5-di(trideuteriomethoxy)phenyl]urea;
1-[1-[dideuterio(1H-pyrrolo[2,3-b]pyridin-3-yl)methyl]indol-4-yl]-3-[5-trifluoromethyl-2-(trideuteriomethoxy)phenyl]urea; or
1-[1-[dideuterio(1H-pyrrolo[2,3-b]pyridin-3-yl)methyl]indol-4-yl]-3-[2-fluoro-5-(trideuteriomethoxy)phenyl]urea.

87. The compound of claim 1, wherein the compound is:
1-(5-chloro-2-fluorophenyl)-3-[1-[dideuterio(1H-pyrrolo[2,3-b]pyridin-4-yl)methyl]indol-4-yl]urea hydrochloride;
1-[1-[dideuterio(1H-pyrrolo[2,3-b]pyridin-4-yl)methyl]indol-4-yl]-3-(m-tolyl)urea hydrochloride;
1-[1-[dideuterio(1H-pyrrolo[2,3-b]pyridin-4-yl)methyl]indol-4-yl]-3-[2-(trideuteriomethoxy)phenyl]urea;
1-[1-[dideuterio(1H-pyrrolo[2,3-b]pyridin-4-yl)methyl]indol-4-yl]-3-[3-(trideuteriomethoxy)phenyl]urea;
1-[1-[dideuterio(1H-pyrrolo[2,3-b]pyridin-4-yl)methyl]indol-4-yl]-3-[4-(trideuteriomethoxy)phenyl]urea;
1-[1-[dideuterio(1H-pyrrolo[2,3-b]pyridin-4-yl)methyl]indol-4-yl]-3-[5-fluoro-2-(trideuteriomethoxy)phenyl]urea
1-[1-[dideuterio(1H-pyrrolo[2,3-b]pyridin-4-yl)methyl]indol-4-yl]-3-[5-chloro-2-(trideuteriomethoxy)phenyl]urea;
1-(5-chloro-2-methoxyphenyl)-3-[1-[dideuterio(1H-pyrrolo[2,3-b]pyridin-4-yl)methyl]indol-4-yl]urea hydrochloride;
1-[1-[dideuterio(1H-pyrrolo[2,3-b]pyridin-4-yl)methyl]indol-4-yl]-3-[5-bromo-2-(trideuteriomethoxy)phenyl]urea;
1-[1-[dideuterio(1H-pyrrolo[2,3-b]pyridin-4-yl)methyl]indol-4-yl]-3-[5-methyl-2-(trideuteriomethoxy)phenyl]urea;
1-[1-[dideuterio(1H-pyrrolo[2,3-b]pyridin-4-yl)methyl]indol-4-yl]-3-[2,5-di(trideuteriomethoxy)phenyl]urea;
1-[1-[dideuterio(1H-pyrrolo[2,3-b]pyridin-4-yl)methyl]indol-4-yl]-3-[5-trifluoromethyl-2-(trideuteriomethoxy)phenyl]urea; or
1-[1-[dideuterio(1H-pyrrolo[2,3-b]pyridin-4-yl)methyl]indol-4-yl]-3-[2-fluoro-5-(trideuteriomethoxy)phenyl]urea.

88. The compound of claim 1, wherein the compound is:
1-[1-[(2-amino-4-pyridyl)dideuteriomethyl]indol-4-yl]-3-[2-(trideuteriomethoxy)phenyl]urea;
1-[1-[(2-amino-4-pyridyl)dideuteriomethyl]indol-4-yl]-3-[3-(trideuteriomethoxy)phenyl]urea;
1-[1-[(2-amino-4-pyridyl)dideuteriomethyl]indol-4-yl]-3-[4-(trideuteriomethoxy)phenyl]urea;
1-[1-[(2-amino-4-pyridyl)dideuteriomethyl]indol-4-yl]-3-[5-fluoro-2-(trideuteriomethoxy)phenyl]urea;
1-[1-[(2-amino-4-pyridyl)dideuteriomethyl]indol-4-yl]-3-[5-chloro-2-(trideuteriomethoxy)phenyl]urea;
1-[1-[(2-amino-4-pyridyl)dideuteriomethyl]indol-4-yl]-3-[5-bromo-2-(trideuteriomethoxy)phenyl]urea;
1-[1-[(2-amino-4-pyridyl)dideuteriomethyl]indol-4-yl]-3-[5-methyl-2-(trideuteriomethoxy)phenyl]urea;
1-[1-[(2-amino-4-pyridyl)dideuteriomethyl]indol-4-yl]-3-[2,5-di(trideuteriomethoxy)phenyl]urea;
1-[1-[(2-amino-4-pyridyl)dideuteriomethyl]indol-4-yl]-3-[5-trifluoromethyl-2-(trideuteriomethoxy)phenyl]urea; or
1-[1-[(2-amino-4-pyridyl)dideuteriomethyl]indol-4-yl]-3-[2-fluoro-5-(trideuteriomethoxy)phenyl]urea.

89. The compound of claim 1, wherein the compound is:
1-[1-[(2-amino-4-pyridyl)-dideuteriomethyl]indolin-4-yl]-3-(5-chloro-2-methoxyphenyl)urea; or
1-[1-[(2-amino-4-pyridyl)-dideuteriomethyl]indolin-4-yl]-3-[5-chloro-2-(trideuteriomethoxy)phenyl]urea.

90. The compound of claim 1, wherein the compound is:
1-[1-[(2-amino-4-pyridyl)methyl]indolin-4-yl]-3-[5-chloro-2-(trideuteriomethoxy)phenyl]urea; or
1-[1-[(2-amino-4-pyridyl)methyl]indol-4-yl]-3-[5-chloro-2-(trideuteriomethoxy)phenyl]urea.

91. The compound of claim 1, wherein the compound is:
1-[5-chloro-2-(trideuteriomethoxy)phenyl]-3-[1-[deuterio(1H-pyrrolo[2,3-b]pyridin-3-yl)methyl]indolin-4-yl]urea;
1-(5-chloro-2-methoxyphenyl)-3-[1-[deuterio(1H-pyrrolo[2,3-b]pyridin-3-yl)methyl]indolin-4-yl]urea;
1-(5-chloro-2-fluorophenyl)-3-[1-[deuterio(1H-pyrrolo[2,3-b]pyridin-3-yl)methyl]indolin-4-yl]urea; or
1-[1-[deuterio(1H-pyrrolo[2,3-b]pyridin-3-yl)methyl]indolin-4-yl]-3-(m-tolyl)urea.

92. The compound of claim 1, wherein the compound is:
1-[5-chloro-2-(trideuteriomethoxy)phenyl]-3-[1-[deuterio(1H-pyrrolo[2,3-b]pyridin-3-yl)methyl]indol-4-yl]urea;
1-(5-chloro-2-methoxyphenyl)-3-[1-[deuterio(1H-pyrrolo[2,3-b]pyridin-3-yl)methyl]indol-4-yl]urea;
1-(5-chloro-2-fluorophenyl)-3-[1-[deuterio(1H-pyrrolo[2,3-b]pyridin-3-yl)methyl]indol-4-yl]urea; or
1-[1-[deuterio(1H-pyrrolo[2,3-b]pyridin-3-yl)methyl]indol-4-yl]-3-(m-tolyl)urea.

93. The compound of claim 1, wherein the compound is:
1-[1-[(2-amino-4-pyridyl)-dideuteriomethyl]indol-4-yl]-3-(5-chloro-2-methoxyphenyl)urea hydrochloride;
1-[1-[(2-amino-4-pyridyl)-dideuteriomethyl]indol-4-yl]-3-[5-chloro-2-(trideuteriomethoxy)phenyl]urea;
1-[5-chloro-2-(trideuteriomethoxy)phenyl]-3-[1-[dideuterio(1H-pyrrolo[2,3-b]pyridin-4-yl)methyl]indolin-4-yl]urea hydrochloride;
1-[5-chloro-2-(trideuteriomethoxy)phenyl]-3-[1-[dideuterio(1H-pyrrolo[2,3-b]pyridin-4-yl)methyl]indol-4-yl]urea hydrochloride;
1-(5-chloro-2-methoxyphenyl)-3-[1-[dideuterio(1H-pyrrolo[2,3-b ]pyridin-4-yl)methyl]indolin-4-yl]urea hydrochloride;
1-(5-chloro-2-methoxyphenyl)-3-[1-[dideuterio(1H-pyrrolo[2,3-b]pyridin-4-yl)methyl]indol-4-yl]urea hydrochloride;
1-(5-chloro-2-fluorophenyl)-3-[1-[dideuterio(1H-pyrrolo[2,3-b]pyridin-4-yl)methyl]indolin-4-yl]urea hydrochloride;
1-(5-chloro-2-fluorophenyl)-3-[1-[dideuterio(1H-pyrrolo[2,3-b]pyridin-4-yl)methyl]indol-4-yl]urea hydrochloride;
1-[1-[dideuterio(1H-pyrrolo[2,3-b]pyridin-4-yl)methyl]indolin-4-yl]-3-(m-tolyl)urea hydrochloride; or
1-[1-[dideuterio(1H-pyrrolo[2,3-b]pyridin-4-yl)methyl]indol-4-yl]-3-(m-tolyl)urea hydrochloride.

94. A pharmaceutical composition comprising a therapeutically effective amount of a compound of claim 1 or a pharmaceutically acceptable salt thereof, and a pharmaceutically acceptable carrier.

95. The composition of claim 94, wherein the composition takes on the forms of injectable, aerosol, cream, gel, capsule, pill, tablet, syrup, eye drop, or ointment.

96. A method for treating a patient with a physical condition mediated by protein kinase, comprising administering to the patient in need thereof a therapeutically effective amount of a compound of claim 1 or a pharmaceutically acceptable salt thereof.

97. The method of claim 96, wherein the protein kinase is VEGFR2, PDGFRβ, EphB4, or c-Met.

98. The method of claim 96, wherein the physical condition is tumor, rheumatoid arthritis, autoimmune disease, acute inflammation, nephritis, diabetic retinitis, psoriasis, or macular degeneration.

99. A method for treating a patient with a physical condition, comprising administering to the patient in need thereof a therapeutically effective amount of a compound of claim 1 or a pharmaceutically acceptable salt thereof, wherein the physical condition is tumor, rheumatoid arthritis, autoimmune disease, acute inflammation, nephritis, diabetic retinitis, psoriasis, or macular degeneration.
